(19) United States
(12) Patent Application Publication
(10) Pub. No.: US 2026/0196341 A1
(43) Pub. Date: Jul. 9, 2026

(54) AI-DRIVEN HEALTHCARE PLATFORM FOR INTEGRATED AND AUTOMATED CARE WORKFLOWS

(71) Applicant: RAVI HARIPRASAD, MD, A PC, Folsom, CA (US)

(72) Inventor: Ravi Hariprasad, Folsom, CA (US)

(21) Appl. No.: 19/425,142

(22) Filed: Dec. 18, 2025

Related U.S. Application Data
(63) Continuation of application No. PCT/US2025/030111, filed on May 20, 2025.
(60) Provisional application No. 63/741,627, filed on Jan. 3, 2025.

(51) Int. Cl. G16H 40/20; G06Q 10/0633; G06Q 30/04; G06Q 40/08; G16H 10/60

(52) U.S. Cl. CPC G16H 40/20; G06Q 10/0633; G06Q 30/04; G06Q 40/084; G16H 10/60; G06Q 2220/123

(57) ABSTRACT

Systems and methods are described for generating a customized care plan for a patient, tracking data and progress of the patient across a service line history over time, generating at least one analytics dashboard, and causing display of the at least one analytics dashboard, wherein the patient information in the at least one analytics dashboard is encrypted and accessible for view according to predefined patient permissions.

### CROSS-REFERENCE TO RELATED APPLICATION

This application claims the priority benefit of U.S. Provisional Patent Application Ser. No. 63/741,627, filed on Jan. 3, 2025, which is herein incorporated by reference in its entirety.

### TECHNICAL FIELD

This disclosure relates generally to the field of healthcare information management systems, and more specifically to AI-driven, automated healthcare platforms.

### BACKGROUND

Mental health disorders have become a critical public health challenge worldwide, with increasing demand for accessible, affordable, and scalable solutions. Traditional mental healthcare models often face limitations such as high costs, fragmented care, and inefficient workflows, making it difficult for patients to receive timely and coordinated treatment. Additionally, the administrative burden on healthcare providers and clinicians further exacerbates the inefficiencies within the system.

### SUMMARY

With the advent of digital health technologies, there is an opportunity to transform healthcare by integrating automation, artificial intelligence (AI), and interoperability into a seamless and patient-centric framework. Conventional digital health platforms, however, frequently lack comprehensive service integration, robust data management, and the ability to personalize care while ensuring compliance with regulatory standards. There is a need for new and useful system and method for generating a patient-specific clinical assessment. In particular, there is a need for systems, devices, and methods that can effectively analyze patient health data, including biometric information, historical medical records, and patient-reported symptoms, to generate personalized clinical insights.

In some aspects, the techniques described herein relate to a computer-implemented method for managing patient care workflows, the method including: generating a customized care plan for a patient stored in a patient registry, the patient registry including patient information including demographic details, payer details, assigned care teams, and service line history; tracking data and progress of the patient across a service line history over time, the tracked data including: at least one service duration, session notes, and billing of a practitioner according to care provided to the patient; generating at least one analytics dashboard including one or more of: patient outcomes, referral effectiveness, and team performance metrics visualized in near real time based at least in part on the customized care plan, the tracked data, and the tracked progress; and causing display of the at least one analytics dashboard, wherein the patient information in the at least one analytics dashboard is encrypted and accessible for view according to predefined patient permissions.

In some aspects, the techniques described herein relate to a method, further including: matching the at least one service duration and the care provided to the patient with one or more Current Procedural Terminology (CPT) codes; providing access to a centralized repository, wherein the centralized repository manages patient assessments, care plans, claims, and billing records; receiving from a clinician interface, billable service entries representing the care provided to the patient; generating, based on the billable service entries, a corresponding Fast Healthcare Interoperability Resources (FHIR)-compliant claim containing patient details, provider information, and the CPT codes matched to the care provided to the patient; validating the claim for completeness and compliance; submitting the claim to a payer or clearinghouse; and receiving and processing claim responses and updating a claim registry.

In some aspects, the techniques described herein relate to a method, wherein validating the claim is performed by cross-referencing the CPT codes with payer policies.

In some aspects, the techniques described herein relate to a method, further including: identifying, within the claim, missing fields, incorrect CPT codes, or payer specific compliance issue; classifying rejected claims based on error severity, distinguishing between minor auto-correctable errors and manually correctable errors; correcting rejected claims based on the classifying, the correcting including automatically generating missing data for the missing fields or performing one or more suggested manual modification classified as a manually correctable error; and resubmitting corrected claims to payers or clearing houses after validation.

In some aspects, the techniques described herein relate to a method, further including: receiving patient referrals and storing, updating and managing the patient referrals within the centralized repository; assigning one or more of the received patient referrals based on provider availability, specialization, and patient needs; triggering follow-ups for pending referrals and flagging unresolved cases for manual intervention associated with one or more of the received patient referrals; and providing a real-time notification that alerts at least one of an assigned specialist, referring clinician, and the patient of the triggered follow-ups.

In some aspects, the techniques described herein relate to a method, further including: receiving an approved claim and generating a corresponding invoice; matching payments received from payers with the generated invoices; identifying adjustments and applying for discrepancies; and flagging and escalating outstanding balances for further action.

In some aspects, the techniques described herein relate to a method, further including: assigning tasks to clinicians based on workload balancing and provider availability; flagging overdue tasks and reassign the overdue task; providing real-time updates on pending patient referrals, claims and patient follow-ups; and alerting users of pending tasks, due dates and urgent follow-ups.

In some aspects, the techniques described herein relate to a method, wherein the tracking of the data includes matching the at least one service duration and the care provided to the patient with one or more Current Procedural Terminology (CPT) codes.

In some aspects, the techniques described herein relate to a method, further including: receiving patient consent preferences; verifying, using the patient registry, consent status before sharing data with one or more third parties; blocking unauthorized access attempts to patient information stored in the patient registry; and generating audit logs for regulatory compliance tracking of the patient registry.

In some aspects, the techniques described herein relate to a computer-implemented system for managing patient care workflows, the system including: a patient registry including patient data and configured to maintain patient demographic details, insurance information, and assigned care teams; a practitioner registry including a plurality of practitioners adapted to provide care to patients in the patient registry; a care plan library storing a plurality of care plan templates configured for dynamic customization for patients in the patient registry; a time tracking and billing module configured to receive practitioner time and generate bills for the care provided to the patients in the patient registry; a clinical workflow engine configured to generate and process claims; a processor; and memory communicably coupled to the processor, wherein the memory stores processor-executable instructions, which when executed by the processor, cause the processor to trigger the clinical workflow engine to link patient records with provider data stored in the practitioner registry to define care team assignments; for each linked patient record: retrieve a care plan from the care plan library; automate document handling in real time for the linked patient record to generate or update the care plan, assessment data, progress reports, and claims records associated with the linked patient record; track service durations for the care provided to the patient in each linked patient record, wherein billable minutes are associated with standardized billing codes; and orchestrate workflow automation for each linked patient record according to predefined rules, the orchestration including generating claims according to the standardized billing codes based on the tracked service durations.

In some aspects, the techniques described herein relate to a system, wherein the processor is further configured to trigger the clinical workflow engine to perform, escalating tasks associated with processing the generated claims, tracking care referrals associated with the generated claims, and generating real-time reports including referral tracking metrics and performance analytics.

In some aspects, the techniques described herein relate to a system, wherein the processor is further configured to trigger the clinical workflow engine to maintain an audit trail for each orchestrated workflow automation, the audit trail being configured for use in compliance tracking of records in the patient registry and the practitioner registry.

In some aspects, the techniques described herein relate to a system, wherein the processor is further configured to trigger the clinical workflow engine to: submit the generated claims in Fast Healthcare Interoperability Resources (FHIR)-compliant formats to external payer systems; verify the generated claims against payer-specific rules, including Current Procedural Terminology (CPT) code restrictions and Medically Unlikely Edits (MUE) limits; and receive, categorize, and process payer responses, facilitating approval tracking, resubmissions for rejected claims, and financial reconciliation.

In some aspects, the techniques described herein relate to a system, wherein the processor is further configured to trigger the clinical workflow engine to: identify rejected claims, categorize the rejected claims based on error type, and trigger automated resubmission workflows after generating corrected claims.

In some aspects, the techniques described herein relate to a system, wherein generating the corrected claims includes: identifying in the rejected claims, missing fields, incorrect CPT codes, or payer-specific compliance issues; classifying rejected claims based on error severity, distinguishing between minor auto-correctable errors and major issues requiring manual review; auto-filling missing data or suggesting manual modifications; and resubmitting the corrected claims to payers or clearinghouses after validation.

In some aspects, the techniques described herein relate to a system, wherein the processor is further configured to trigger the clinical workflow engine to: store, update, and manage patient referrals within a centralized database; dynamically assign referrals based on provider availability, specialization, and patient needs; trigger follow-ups for pending referrals and flag unresolved cases for manual intervention; and alert assigned specialists, referring clinicians, and patients of referral status updates.

In some aspects, the techniques described herein relate to a system, wherein the processor is further configured to trigger the clinical workflow engine to notify clinicians when pending referrals exceed a predefined wait time.

In some aspects, the techniques described herein relate to a system, wherein the processor is further configured to trigger the clinical workflow engine to: create invoices based on approved claims, including billed amounts, due dates, and payer details; reconcile received payments with corresponding invoices using Explanation of Benefits (EOB) data; process partial payments, denials, and underpayments, updating financial records accordingly; and flag unpaid invoices and trigger automated follow-ups for overdue payments.

In some aspects, the techniques described herein relate to a non-transitory computer-readable storage medium storing executable program instructions that, when executed by a computing system, cause the computing system to perform operations including: managing patient records within a patient registry, wherein patient demographic details, payer information, and care team assignments are maintained; storing provider data in a practitioner registry, wherein practitioner credentials, specializations, and availability are updated dynamically; retrieving care plans from a care plan library, wherein pre-configured templates are modified in response to patient-specific conditions; automating document management, wherein patient assessments, billing records, and progress reports are stored and updated; tracking billable service durations, wherein service times are mapped to standardized medical codes for claims processing; executing automated workflows, wherein claims are processed, referrals are managed, and overdue tasks trigger alerts; providing real-time analytics, wherein dashboards track patient outcomes, referral efficiency, and team performance; and securing patient data according to regulatory compliance rules and based on encryption, role-based access control, and audit logging.

In some aspects, the techniques described herein relate to a non-transitory computer-readable storage medium, wherein real-time alerts notify care coordinators of overdue patient assessments or incomplete documentation.

In some aspects, the techniques described herein relate to a non-transitory computer-readable storage medium, wherein the claims processing further includes: escalating tasks associated with processing the claims, tracking care referrals associated with the claims, and generating real-time reports including referral tracking metrics and performance analytics.

### BRIEF DESCRIPTION OF THE DRAWINGS

There is a need for new and useful system and method for generating a patient-specific clinical assessment. In particular, there is a need for systems, devices, and methods that can effectively analyze patient health data, including biometric information, historical medical records, and patient-reported symptoms, to generate personalized clinical insights.

FIG. 1A illustrates a block diagram of an example clinical assessment system for generating a patient-specific clinical assessment, in accordance with an embodiment of the present disclosure.

FIG. 1B illustrates a functional block diagram of the example clinical assessment system, in accordance with an embodiment of the present disclosure.

FIG. 2 illustrates an example flow diagram of processing patient health data, in accordance with an embodiment of the present disclosure.

FIG. 3 illustrates an example flow diagram of training and initialization of the trained LLM, in accordance with an embodiment of the present disclosure.

FIG. 4 illustrates an example flow diagram of dynamically updating the trained LLM, in accordance with an embodiment of the present disclosure.

FIG. 5 illustrates an example flow diagram of semantic analysis and context-aware processing, in accordance with an embodiment of the present disclosure.

FIG. 6 illustrates an example flow diagram of clinical workflow and decision support, in accordance with an embodiment of the present disclosure.

FIG. 7 illustrates an example flow diagram of task allocation and workflow management, in accordance with an embodiment of the present disclosure.

FIG. 8 illustrates an example dual-panel diagram depicting a patient interface and a clinician interface, in accordance with an embodiment of the present disclosure.

FIG. 9 illustrates an example schematic diagram depicting encryption of the patient health data, in accordance with an embodiment of the present disclosure.

FIG. 10 illustrates an example flow diagram of testing, deployment, and updates of the clinical assessment system, in accordance with an embodiment of the present disclosure.

FIG. 11 illustrates an example architectural diagram of cloud-based data management, in accordance with an embodiment of the present disclosure.

FIG. 12 illustrates an example flow diagram of real-time data handling within the clinical assessment system, in accordance with an embodiment of the present disclosure.

FIG. 13 illustrates an example schematic diagram of the patient interface, in accordance with an embodiment of the present disclosure.

FIG. 14 illustrates an example schematic diagram of the clinician interface, in accordance with an embodiment of the present disclosure.

FIG. 15 illustrates an example flow diagram of data security and privacy protocol within the clinical assessment system, in accordance with an embodiment of the present disclosure.

FIG. 16 illustrates an example flow diagram of interactive prompting and data capture process during patient intake, in accordance with an embodiment of the present disclosure.

FIG. 17 illustrates an example flow diagram of analyzing clinical data and generating treatment recommendations within the clinical assessment system, in accordance with an embodiment of the present disclosure.

FIG. 18 illustrates an example flow diagram of communication flow and notification within the clinical assessment system, in accordance with an embodiment of the present disclosure.

FIG. 19 illustrates an example architectural diagram depicting patient-centered care delivery, in accordance with an embodiment of the present disclosure.

FIG. 20 illustrates the interaction between different registries within the Patient Care Management platform, demonstrating the various components working together to streamline patient onboarding, care coordination, and billing, in accordance with an embodiment of the present disclosure.

FIG. 21 illustrates an example data flow diagram of aggregation and processing of data from care activities, task performance, claims data, and patient outcomes into clinical insights via business intelligence dashboards, in accordance with an embodiment of the present disclosure.

FIG. 22 illustrates an example flow diagram of the phases of a patient journey within the Patient Care Management platform, in accordance with an embodiment of the present disclosure.

FIG. 23 illustrates an example flow diagram of the structured movement of data across various workflows, enabling seamless coordination, real-time insights, and operational efficiency, in accordance with an embodiment of the present disclosure.

FIG. 24 illustrates an example flow diagram of the flow of the patient referral to care team assignment, in accordance with an embodiment of the present disclosure.

FIG. 25 illustrates an example flow diagram for the patient health data processing within the clinical assessment system, is illustrated, in accordance with an embodiment of the present disclosure.

FIG. 26 illustrates an example flow diagram of the claim processing workflow, in accordance with an embodiment of the present disclosure.

FIG. 27 illustrates an example flow diagram for a patient referral tracking and coordination workflow, in accordance with an embodiment of the present disclosure.

FIG. 28 illustrates an example data flow diagram depicting the integration of various data streams into business intelligence dashboards for clinical insights, in accordance with an embodiment of the present disclosure.

FIG. 29 illustrates an example flow diagram of the data structure of the patient care management platform, in accordance with an embodiment of the present disclosure.

FIG. 30 illustrates an example flow diagram of the patient referral to care team assignment, in accordance with an embodiment of the present disclosure.

FIG. 31 illustrates an example workflow diagram for assessment data processing, showcasing the end-to-end handling of patient-submitted assessments to generate clinical insights, in accordance with an embodiment of the present disclosure.

FIG. 32 illustrates an example workflow diagram for care plan development and monitoring, depicting the processes for creating, updating, and tracking patient-specific care plans dynamically, in accordance with an embodiment of the present disclosure.

FIG. 33 illustrates an example flow diagram of a claim processing workflow, in accordance with an embodiment of the present disclosure.

FIG. 34 illustrates an example flow diagram of a referral tracking and specialist coordination workflow, in accordance with an embodiment of the present disclosure.

FIG. 35 illustrates an example workflow diagram for the seamless data flow into dashboards designed for real-time insights, in accordance with an embodiment of the present disclosure.

FIG. 36 illustrates an example workflow for collecting, processing, and analyzing patient feedback, in accordance with an embodiment of the present disclosure.

FIG. 37 illustrates an example flow diagram of a workflow that automates time tracking for practitioners, ensuring accurate payroll processing while integrating with the claims and billing system, in accordance with an embodiment of the present disclosure.

FIG. 38 illustrates an example flow diagram of a process for generating claims billing notes and progress visit notes, in accordance with an embodiment of the present disclosure.

FIG. 39 illustrates an example flow diagram of a process of capturing, validating, and submitting billing data to ensure accurate claim generation and seamless payer integration, in accordance with an embodiment of the present disclosure.

FIG. 40 illustrates an example flow diagram of a workflow that automates the assignment of Current Procedural Terminology (CPT) codes to healthcare services, ensuring compliance with billing regulations and seamless claim processing, in accordance with an embodiment of the present disclosure.

FIG. 41 illustrates an example flow diagram of an automated claim submission and tracking process, in accordance with an embodiment of the present disclosure.

FIG. 42 illustrates an example flow diagram of an automated error handling and resubmission process for claim rejections, in accordance with an embodiment of the present disclosure.

FIG. 43 illustrates an example flow diagram of the automated claim submission and status update process, in accordance with an embodiment of the present disclosure.

FIG. 44 illustrates an example flow diagram of the financial reconciliation workflow, in accordance with an embodiment of the present disclosure.

FIG. 45 illustrates an example use case scenario for the comprehensive management of a patient, in accordance with an example embodiment of the present disclosure.

FIG. 46 illustrates an example scenario, highlighting a care plan adjustment process triggered by monitoring patient progress and AI-based predictions, in accordance with an example embodiment of the present disclosure.

FIG. 47 illustrates an example flow chart of the patient consent management workflow, in accordance with an embodiment of the present disclosure.

FIG. 48 illustrates a flow chart for managing patient care workflow, in accordance with an embodiment of the present disclosure.

FIG. 49 illustrates a flow chart of a method for generating a patient-specific clinical assessment, in accordance with an embodiment of the present disclosure.

FIG. 50 illustrates an example flow chart of a method for utilizing the clinical assessment system for a patient managing diabetes.

FIG. 51 illustrates an example flow chart of a method for utilizing the clinical assessment system for a patient managing a chronic disease.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology, will now be described in connection with various embodiments. The inclusion of the following embodiments is not intended to limit the disclosure of these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In conventional healthcare, interactions between patients and clinicians are constrained by specific time limitations imposed by insurance reimbursement models and industry productivity standards. These time constraints, combined with increasing clinician workloads, prevent thorough health-related patient assessments while simultaneously requiring comprehensive documentation. Administrative requirements further impede meaningful patient-clinician communication. Consequently, clinicians often obtain incomplete patient histories, potentially resulting in suboptimal or inappropriate treatment plans. The challenges of data management are further exacerbated when implementing personalized healthcare approaches, which inherently demand more comprehensive patient information and more complex data analysis. The psychiatric care sector faces unprecedented challenges stemming from a quantifiable shortage of specialized practitioners. This deficiency directly causes restricted availability of essential mental health services and/or other health services, creating a measurable disparity between patient needs and healthcare system capacity. The resulting service gap prevents timely intervention for many patients that would benefit from specialized psychiatric assessment and treatment and/or specialized health-related assessments and treatments, with particularly severe impacts in underserved communities where specialist-to-patient ratios fall below recommended clinical guidelines. Unlike conventional healthcare assessment or billing solutions, the systems and methods described herein encompass a unified, orchestrating platform wherein clinical and operational data streams mutually adapt, supporting truly closed-loop, multi-specialty care.

While mental health care examples are utilized in some examples described herein, the systems and methods described herein can be adapted for any specialty or subspecialty of health care. For example, in some embodiments, specialized modules for other health care examples (e.g., cardiology care, diabetes care, endocrinology care, geriatric care, pediatric care, neurologic care, oncology care, disease management care, primary health care, or other health care) may be utilized with the systems described herein. In particular, other health care specialties can be utilized with the healthcare platform including, but not limited to the clinical workflow engine, the real time analytics, claims, and care coordination described herein. Further, although specific mental health use cases (e.g., screening instruments, therapy codes) are described in detail, these are representative. Comparable approaches apply for any standardized assessment or healthcare codes, such as those used for oncology, cardiology, endocrine disorders, or the like. In general, the codes described herein may pertain to any standardized procedure code, billing code, or code representative of a service event or a line item for a healthcare procedure, including, but not limited to, region-specific or payer-specific equivalents.

Conventional systems such as Electronic Health Records (EHRs) and other basic automation tools try to solve some of these problems but do not offer a total solution. Generally, these systems have failed to utilize patient data to streamline the treatment and optimize clinical workflow. Clinician burnout that is driven by administrative workload underscores the long felt need for an improved solution that can streamline tasks like data collection from patients, data entry, data analysis, assessment and tracking of billing events, assessment and tracking of practitioner time entry, events, and schedules, generation and assessment of referral plans, generation and assessment of care plans, and assessment of patient progress.

The present disclosure addresses these conventional challenges by providing an innovative digital healthcare platform designed to optimize health service delivery. By integrating health care such as physical health care, psychiatric care (and/or other medical specialties), lifestyle interventions, chronic care management, and assessment services into a unified system, the platform enables holistic and continuous patient care. The platform is built on a scalable digital infrastructure, leveraging cloud-based technologies and AI-driven insights to enhance clinician workflows, improve patient engagement, and ensure secure data interoperability.

Through automation-driven workflows, AI-powered analytics, and compliance with healthcare data exchange standards, the platform described herein streamlines administrative processes, reduces clinician burnout, and improves treatment outcomes. By bridging technology with compassionate care, the platform redefines the accessibility and efficiency of health services, setting a new standard for the future of digital healthcare. The system and health modules described herein may be powered by artificial intelligence or other computing technology to execute cohort analytics, risk stratification, and real-time (or near real-time) outcome-triggered intervention recommendations that may be adaptive across any care specialty. In general, workflow logic and system configuration may be continuously or periodically improved in response to observed patient outcomes, feedback, and/or regulatory landscape.

At a high level, the systems and methods described herein represent a patient-centered mental and/or physical healthcare platform designed to address the growing challenges of accessibility, affordability, and scalability in mental and/or physical health services. The systems are built on a robust and scalable digital framework, that seamlessly integrates patient care (e.g., psychiatric care, lifestyle interventions, chronic care management, and assessment services) into a unified ecosystem. In some embodiments, the systems and methods described herein may manage patient care workflows, billing workflows, and clinician workflows and may provide output for such workflows in a user interface. Such systems may function as (or integrate with) other modules for generating a patient-specific clinical assessment of a patient journey, for example. In some embodiments, the systems and methods described herein may provide an integrated system of patient care and/or tracking, clinician management, billing management, and referral management. In some embodiments, the systems and methods described herein may provide health indicator monitoring along with patient journey assessments.

The systems and methods described herein solve the technical problem of efficiently processing and analyzing complex, heterogeneous patient data, CPT code data (or other data indicating a standardized procedure code or code representative of a service event or a line item for a healthcare procedure), clinician data, and billing data, including unstructured text, biometric information, and historical medical records, to generate patient-specific clinical assessments and insights. By utilizing a trained LLM within a cloud-based environment, the systems and methods address technical challenges such as real-time data normalization, semantic understanding of medical information, and secure integration with electronic medical records (EMR) systems. This approach reduces the computational overhead associated with traditional manual processes which improves the accuracy and timeliness of clinical recommendations and enables unified interoperability across disparate healthcare systems.

In particular, the technical problem sought to be solved by the present disclosure is to provide a system and method for generating at least one analytics dashboard that includes patient outcomes, referral effectiveness, and team (e.g., clinician) performance metrics in near real time. The systems and methods described herein may function to integrate heterogeneous patient data into a unified framework, utilizing advanced artificial intelligence (AI) techniques, including LLMs, to deliver accurate, real-time diagnostic support, personalized treatment recommendations, billing and claim generation with CPT code (or other standardized procedure code or code representative of a service event or a line item for a healthcare procedure) mapping analysis while ensuring compliance with healthcare data security and interoperability standards.

The systems and methods described herein can be used to generate patient journey data covering a lifecycle of patient management, beginning with the referral phase and extending through service line workflows, continuous progress monitoring, and iterative care plan adjustments to ensure optimal healthcare outcomes for the patient. The patient journey data may include one or more of referral intake data, pre-assessment data, multi-service treatment workflow data, continuous progress tracking data, and iterative care modification data to ensure optimal patient outcomes. By dynamically managing practitioner assignments, streamlining service coordination, and implementing automated intervention mechanisms, the systems and methods described herein may improve care delivery efficiency over conventional systems while maintaining a patient-centric, scalable, and outcome-focused care management model. In some embodiments, the systems and methods described herein may use patient journey data to determine particular lifestyle interventions in which to embed into patient care plans, allowing psychiatrists, health coaches, and care teams to work together in real-time to monitor progress.

Some conventional systems and/or methods may utilize static rule-based algorithms or predefined templates for patient assessment and clinical data processing that fail to embrace the dynamic and adaptive needs of modern personalized healthcare. A potential drawback with such conventional solutions may include limited flexibility in adapting to heterogeneous data sources, inability to provide context-aware clinical insights, and a lack of real-time responsiveness to patient-specific changes or clinician inputs. Thus, the devices, methods, and/or MOTs described herein may provide an improvement over conventional solutions by employing an LLM trained to process and analyze diverse patient health data dynamically, utilizing advanced natural language processing techniques for data normalization, and incorporating retrieval-augmented generation (RAG) techniques for enhanced clinical recommendations. These improvements enable personalized, adaptive, and contextually relevant patient care while reducing administrative burdens on clinicians and enhancing interoperability with existing healthcare infrastructure.

In some embodiments, the systems and methods described herein may provide health indicator monitoring. For example, the method may function to proactively track and respond to patient health risks through automated data collection and intervention processes. The method may capture health data from multiple sources, including patient self-reporting, device integrations, and clinician updates, which are logged into a centralized health indicators registry. An automated validation system may check incoming data against predefined clinical thresholds for indicators such as blood pressure, blood glucose levels, and/or mental health screening scores. For example, a claim may be validated by cross-referencing a CPT code (or other standardized mental or physical procedure code or code representative of a service event or a line item for a healthcare procedure) with particular payer policies. When a measurement exceeds normal parameters (e.g., predefined per patient or per population of patients), the system automatically triggers a critical flag and initiates a multi-stage notification protocol. Assigned clinicians receive immediate alerts detailing the patient's critical health information and recommended actions. If no clinician response occurs within a specified timeframe, the case escalates to management with high-priority notifications sent through SMS and/or email. Patients with critical indicators receive automated communications prompting immediate medical attention, and a follow-up consultation is scheduled. The method may be is supported by application programming interface dashboards that enable care teams and managers to monitor aggregate data, track response effectiveness, and identify emerging health trends across the patient population.

The method may further include generating, by the processor, a set of interactive prompts for a patient interface based on the identified clinical insights and/or monitoring output. The set of interactive prompts may be used to obtain additional information associated with the patient. The method may further include receiving, by the processor, a set of patient responses responsive to the generated set of interactive prompts. The method may further include displaying, by the processor, the clinical insights, health indicators, patient journey indicators and/or the set of patient responses on a clinician interface.

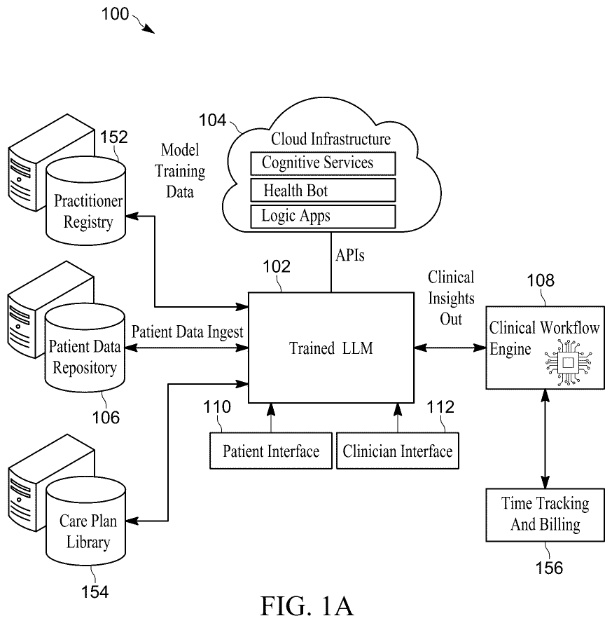

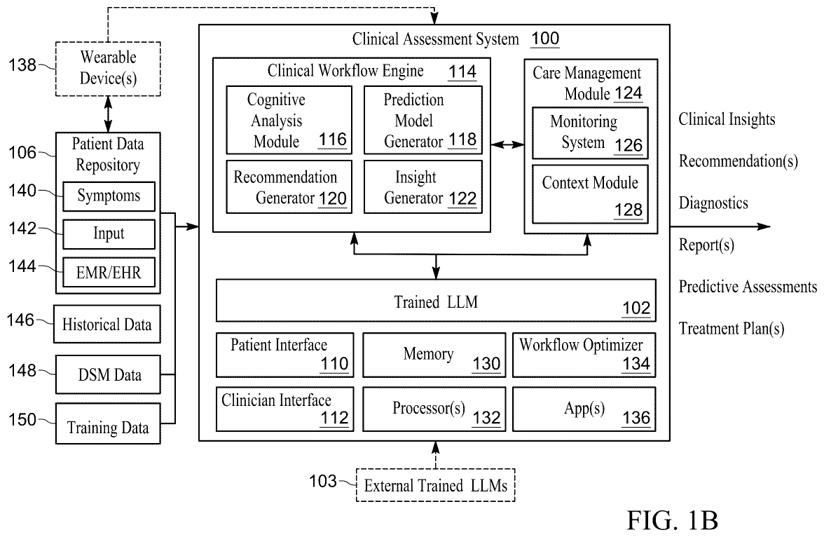

Referring now to FIG. 1A, a block diagram of an example clinical assessment system for generating a patient-specific clinical assessment, is illustrated, in accordance with an embodiment of the present disclosure. The clinical assessment system 100 may integrate advanced AI technologies, specifically a trained LLM 102, practitioner registry 152, patient data repository 106, and care plan library 154 to analyze patient health data and provide clinical insights. In addition, the system 100 may interface with clinical workflow engine 108 and/or time tracking and billing 156 system for claims, billing, and clinician time tracking.

In some embodiments, the system 100 represents a comprehensive mental and/or overall health care platform designed to integrate physical health care, psychiatric care, psychotherapy, lifestyle interventions, and continuous patient monitoring. The system 100 may be built around a structured framework, with a focus on integrating service lines, care plans, testing, and monitoring protocols. These components ensure that patients receive comprehensive, coordinated care at every step of their health journey. The system 100 may be used to automate workflows, streamline care coordination, and provide real-time insights into patient outcomes. The system 100 provides a scalable and adaptable structure, supporting continuous improvements in patient care by integrating lifestyle psychiatry, wellness coaching, and mindfulness programs into its core features, supporting the holistic care. Through automated workflows, dynamic role management, and comprehensive reporting, the system 100 ensures patients receive continuous, personalized, and integrative care across both clinical and wellness service lines.

The clinical assessment system 100 may operate within a cloud-based infrastructure 104. The cloud-based infrastructure 104 may provide one or more services such as cognitive services, health bots, and logic apps, which collectively support AI processing, natural language processing, and workflow automation. The trained LLM 102 may serve as a central processing unit of the clinical assessment system 100 and may be trained on a comprehensive healthcare knowledge database (not shown) that may include but is not limited to, psychiatric research, historical patient data, treatment outcomes, and medical literature. The trained LLM 102 may analyze received patient health data corresponding to a patient, which may include, but is not limited to patient biometric data (e.g., heart rate, blood pressure, oxygen saturation), patient historical medical data (e.g., previous diagnoses, prescribed medications, surgical history), patient journey data, and patient-reported symptoms (e.g., fatigue, chest pain, difficulty breathing). The patient health data may be received from, but is not limited to, a patient data repository 106 and may be processed through Application Programming Interfaces (APIs) connecting the patient data repository 106 to the trained LLM 102. The patient data repository 106 may include one or more input sources that may include but are not limited to, electronic health records, wearable devices, or patient self-reports.

The practitioner registry 152 may store practitioner data (e.g., clinician data/clinic data) that tracks provider credentials, specialties, and role assignments, dynamically linking care team members to patients based on determined service needs. The patient data repository 106 stores patient data, including demographics, payor information, and care history, ensuring that all care plans and service line engagements are documented. The care plan library 154 stores preconfigured templates for evidence-based care plans, enabling rapid customization and deployment.

The trained LLM 102 may perform contextual processing of the patient health data of patient data repository 106, data from practitioner registry 152, and/or data from care plan library 154 to generate clinical insights, including potential diagnoses, prioritized patient conditions, treatment recommendations, and patient monitoring over a journey of a patient over time. The generated clinical insights, which include potential diagnoses, prioritized patient conditions, and treatment recommendations, may be communicated to the clinical workflow engine 108 for further processing and operational integration. The clinical assessment system 100 may interface with one or more user components, for example, a patient interface 110 and a clinician interface 112. The patient interface 110 enables the patient to complete structured intake forms, receive interactive health prompts, and engage interactively with the trained LLM 102 through dynamically generated prompts. The interactive prompts may be generated based on the clinical insights generated by the trained LLM 102 and may be contextualized to gather additional patient-specific information.

The clinician interface 112 may provide healthcare providers with real-time access to clinical insights, patient health data, and workflow management tools. Through the clinician interface 112, clinicians may review the LLM-generated recommendations, validate diagnoses, and tailor treatment plans based on their professional judgment. The clinician interface 112 may also enable seamless synchronization of clinician-reviewed data with existing EMR systems. In some embodiments, the clinician interface 112 may be integrated with time tracking and billing system 156 through the clinical workflow engine 108.

The clinical assessment system 100 incorporates a care plan library 154, which houses standardized treatment protocols and personalized care pathways for various medical conditions. The care plan library 154 ensures that treatment recommendations generated by the trained LLM 102 align with evidence-based medical guidelines. The trained LLM 102 dynamically retrieves relevant care plans from the library 154 to support clinical decision-making and improve patient outcomes.

Additionally, the system maintains the practitioner registry 152, which serves as a database of licensed healthcare providers, their specializations, and professional credentials. The practitioner registry 152 is utilized by both the trained LLM 102 and the clinical workflow engine 108 to assign patient cases to relevant healthcare provider based on expertise, availability, and geographic proximity. This ensures personalized and efficient patient care delivery.

The clinical workflow engine 108 may be integrated with the time tracking and billing system 156, which automates service documentation, provider time tracking, and financial transactions. When a clinician reviews AI-generated insights, validates diagnoses or modifies treatment plans, the time tracking and billing system 156 logs the corresponding actions and calculates billable hours or reimbursable services. This integration ensures that healthcare providers receive accurate compensation while maintaining compliance with insurance requirements and medical billing standards.

The patient health data processing pipeline incorporates multiple stages, including data normalization, metadata augmentation, and interoperability mapping to standardized coding systems such as SNOMED CT and LOINC. Using natural language processing (NLP), the trained LLM 102 contextualizes patient data and enriches it with metadata attributes (e.g., timestamps, locations, and categorical classifications). The system further enhances clinical assessment accuracy through Retrieval-Augmented Generation (RAG) techniques, enabling the LLM to retrieve and dynamically integrate relevant data from the healthcare knowledge database. This approach ensures that clinical recommendations remain evidence-based and up-to-date. Additionally, the trained LLM 102 is designed for continuous learning and adaptation, allowing it to refine its assessment capabilities based on new medical research, patient outcomes, and clinician feedback. The integration of time tracking and billing system 156 ensures that patient care workflows remain efficient and financially accountable.

Referring now to FIG. 1B, a functional block diagram of the example clinical assessment system, is illustrated, in accordance with an embodiment of the present disclosure. The clinical assessment system 100 integrates various hardware and software components to enable efficient data processing. The clinical assessment system 100 may include but is not limited to, the trained LLM 102, the clinical workflow engine 108, the patient interface 110, the clinician interface 112, a care management module 124, a memory 130, one or more processors 132, a workflow optimizer 134, and one or more applications 136.

The processor 132 may include one or more processors, such as central processing units (CPUs), graphics processing units (GPUs), or specialized accelerators designed for machine learning tasks. The one or more processors may include one or more devices capable of executing instructions stored by the memory 130, to perform operations and/or communications amongst systems, engines, modules, and/or devices described herein. The memory 130 may include one or more non-transitory computer-readable storage media, such as solid-state drives (SSDs), dynamic random-access memory (DRAM), or flash storage devices. The memory 130 may store instructions and data that are usable in combination with processor 132 to execute the processes and/or algorithms described herein as well as to execute or interface with trained LLM 102. The memory 130 may also function to store or have access to the trained LLM 102.

The trained LLM 102 is an analytical component of the clinical assessment system 100 and operates as an advanced LLM or other machine learning framework. The trained LLM 102 may be implemented using frameworks such as TensorFlow®, PyTorch®, or other machine learning platforms, and may operate locally or in a cloud-based environment. Alternate embodiments may include multiple AI/ML models to handle specialized tasks, such as predictive or natural language processing. In some embodiments, the trained LLM 102 may be trained on training data 150 received from the healthcare knowledge database (not shown). The training data 150 includes but is not limited to, psychiatric research, historical patient data, treatment outcomes, and medical literature to ensure that the trained LLM 102 is well-versed in both theoretical and practical medical knowledge. To achieve this, the trained LLM 102 employs RAG techniques, which allow it to dynamically retrieve relevant data from connected repositories such as the historical medical data 146 and Diagnostic and Statistical Manual of Mental Disorders (DSM) data sources 148. Other health data sources are of course accessible to the system 100 depending on the particular health care being addressed for a patient. The retrieved information is then combined with the patient data to generate insights that are both comprehensive and individualized.

The clinical workflow engine 108 may include one or more modules or a plurality of modules, including at least a cognitive analysis module 116, a prediction model generator 118, a recommendation generator 120, and an insight generator 122. These modules collectively manage the processing of the patient health data. In some embodiments, the clinical workflow engine 108 may include additional modules for advanced analytics or be integrated with external systems for multi-department coordination.

The care management module 124 includes a monitoring system 126 and a context module 128 to support the real-time tracking of patient progress and the contextualization of data. These sub-modules work in tandem with the clinical workflow engine 108 to ensure personalized and adaptive patient care. Alternate embodiments of the care management module 124 may incorporate predictive monitoring capabilities or AI-driven alerts for high-risk scenarios.

The patient interface 110 represents a patient-centric platform designed to interact directly with patients. The patient interface 110 allows for the collection of patient-reported symptoms, displays clinical insights, and dynamically adapts interactive prompts based on analysis of the trained LLM 102. The patient interface 110 may be implemented as a web-based application, a mobile app, or integrated with the wearable devices 138.

The clinician interface 112 is tailored for healthcare providers or clinicians to offer access to patient health data, LLM-generated insights, and workflow management tools. The clinician interface 112 enables clinicians to review, validate, and update care plans in real-time. The clinician interface 112 may support integration with EMR 144 and may include customization options for individual provider workflows.

The applications 136 within the clinical assessment system 100 provide supplementary functionalities, such as task automation, data visualization, and remote access to the clinical assessment system 100. These applications 136 can operate on various hardware platforms, including computing devices, desktops, tablets, and mobile devices.

Input sources may include wearable devices 138 and the patient data repository 106, which may include patient-reported symptoms 140, input 142, and EMR 144. The input sources may also include historical medical data 146, and DSM data sources 148. These input sources supply real-time or historical data, which is processed by the clinical assessment system 100 for patient analysis. The clinical assessment system 100 may also include training data 150 for continuous refinement of the trained LLM 102.

In some embodiments, the system 100 may execute a computer-implemented method for managing patient care workflows. The method may include generating a customized care plan for a patient stored in a patient registry. The patient registry may include patient information including, but not limited to, demographic details, payer details, assigned care teams, and service line history. The method may include tracking data including, but not limited to, at least one service duration, session notes, and billing of a practitioner when care is provided to the patient. The method may further include matching the at least one service duration and the care provided to the patient with one or more CPT codes (or other standardized procedure code or code representative of a service event or a line item for a healthcare procedure) and may track progress of the patient across the service line history over time. The method may further include generating at least one analytics dashboard. The analytics dashboard(s) may include functionality to generate and display any combination of patient outcomes, referral effectiveness, and team performance metrics using one or more images, text, visualizations, or the like, and may do so in near real time based at least in part on the customized care plan, the tracked data, and the tracked progress. The method may trigger display of the at least one analytics dashboard automatically and/or responsive to a request from a patient, clinician, or other user with approved access to patient data being displayed. The patient information in the at least one analytics dashboard is generally encrypted and accessible for view according to predefined patient permissions.

By way of a non-limiting example, the patient interface 110, the wearable devices 138, or other input sources may provide patient health data corresponding to a patient, to the clinical workflow engine 108 for further processing. In some embodiments, the patient health data may include but is not limited to, one or more of biometric data from the wearable devices 138, historical medical data 146, and patient-reported symptoms 140. The clinical workflow engine 108 may receive the patient health data. The cognitive analysis module 116 of the clinical workflow engine 108 may normalize the patient health data using natural language processing techniques and data standardization techniques. The context module 128 within the care management module 124 may further augment the normalized data with metadata corresponding to the patient health data. In some embodiments, the metadata may be received from one or more of the input sources, such as the wearable devices 138, electronic health records from the EMR 144, or the patient-reported symptoms 140. The metadata may include temporal attributes, locational attributes, and/or categorical attributes corresponding to the patient health data. The cognitive analysis module 116 may further map the normalized and/or augmented data to a standardized medical coding system, such as SNOMED CT or LOINC. The workflow engine 108 may encrypt the patient health data using an end-to-end encryption protocol to ensure data privacy and security.

The trained LLM 102, as part of the clinical workflow engine 108, may analyze the patient health data to generate clinical insights. The insight generator 122 performs this analysis by using the contextual processing of the trained LLM 102, which personalizes the identified clinical insights. The contextual processing may be based, at least in part, on historical medical data 146, real-time updates from the wearable devices 138 associated with the patient, and diagnostic criteria from the DSM data sources 148. The insight generator 122 using the trained LLM 102 identifies clinical insights, which may include, but are not limited to, potential diagnoses, treatment recommendations, and prioritized patient conditions. The monitoring system 126 of the care management module 124 may assist in dynamically tracking symptom progression or health trends to further refine the identified clinical insights.

The recommendation generator 120 generates a set of interactive prompts for the patient interface 110 based on the identified clinical insights. These interactive prompts may be used to obtain additional information associated with the patient. The recommendation generator 120, in conjunction with the trained LLM 102, dynamically adapts the set of prompts using a decision-tree algorithm based on the patient responses. The patient interface 110 receives and transmits these responses back to the clinical workflow engine 108, where the insight generator 122 processes the input 142 to refine clinical insights. The updated clinical insights and patient responses are then displayed on the clinician interface 112 (e.g., via one or more dashboards described herein), allowing healthcare providers to validate or modify the care recommendations.

The processor 132 may further generate a set of interactive prompts for a patient interface 110 based on the identified clinical insights, the set of interactive prompts being configured to obtain additional information associated with the patient. The processor 132 may further dynamically adapt the set of prompts based on the set of patient responses, using a decision-tree algorithm implemented by the trained LLM 102. The processor 132 may further receive a set of patient responses responsive to the generated set of interactive prompts. The processor 132 may further display the clinical insights and the set of patient responses on the clinician interface 112.

The trained LLM 102 is dynamically updated with the patient health data and clinician feedback using the workflow engine 108 to improve diagnostic accuracy and treatment recommendations over time. The clinical workflow engine 108 synchronizes the identified clinical insights and clinician-reviewed data with the EMR 144 to maintain up-to-date patient records.

In a non-limiting example, consider a patient, Sarah, a 45-year-old with a history of Type 2 diabetes, generalized anxiety disorder (GAD), and mild hypertension. Sarah uses the wearable device 138 to track her physical activity, glucose levels, and heart rate. Additionally, she provides patient-reported symptoms 140 such as fatigue and occasional dizziness through the patient interface 110. This example demonstrates how the clinical assessment system 100 processes her health data (i.e., patient health data) to generate personalized clinical insights and care recommendations.

Sarah's wearable device transmits real-time biometric data, including her glucose levels, heart rate variability, and daily step count, to the clinical workflow engine 108. In parallel, Sarah logs her fatigue severity and dietary intake (i.e., input 142) using the patient interface 110, while her historical medical data 146, such as past treatments and lab results, is retrieved from EMR 144. Additionally, the clinical assessment system 100 incorporates DSM data sources 148 to cross-reference diagnostic criteria for her anxiety symptoms.

The cognitive analysis module 116 within the clinical workflow engine 108 normalizes this patient health data using natural language processing (NLP) and data standardization techniques. Metadata such as the time of day, location, and context of Sarah's logged symptoms are appended by the context module 128 in the care management module 124 to ensure that the data is enriched with temporal, locational, and/or categorical attributes. The normalized and augmented data is then mapped to a standardized medical coding system, such as SNOMED CT, for interoperability.

The trained LLM 102, integrated with the clinical workflow engine 108, analyzes Sarah's health data (i.e., patient health data) to identify clinical insights. The insight generator 122 processes this patient's health data using contextual processing techniques, utilizing her historical medical records and real-time updates from her wearable device 138. For Sarah, the insight generator 122 identifies a potential diagnosis of prediabetic neuropathy based on her elevated glucose levels and reported symptoms of fatigue and dizziness. The insight generator 122 also identifies a recommendation for cognitive behavioral therapy (CBT) to manage her anxiety, tailored to DSM data sources 148. The insight generator 122 also identifies a prioritized condition list with her fluctuating glucose levels flagged as urgent for immediate intervention. The monitoring system 126 tracks Sarah's symptom progression, dynamically updating the clinical insights to reflect trends in her glucose levels and heart rate.

The recommendation generator 120 generates interactive prompts for the patient interface 110 based on the generated clinical insights. Sarah is asked to answer the interactive prompts about her dietary habits, stress levels, and sleep quality. The trained LLM 102, in conjunction with the recommendation generator 120, dynamically adapts these interactive prompts using a decision-tree algorithm to ensure that the questions are personalized and relevant. For example, if Sarah indicates high-stress levels, the clinical assessment system 100 generates additional prompts about recent life changes or work-related stressors. Sarah's responses are transmitted back to the clinical workflow engine 108, where the insight generator 122 refines its recommendations based on her inputs. The refined clinical insights and Sarah's responses are displayed on the clinician interface 112. The provider sees a flagged alert for immediate glucose level management. The provider also sees a recommendation to adjust Sarah's dietary plan and increase her physical activity. The provider also sees a proposed referral to a therapist for CBT sessions. The clinician interface 112 provides an interactive dashboard that allows the provider to modify care plans in real-time and synchronize the updates with Sarah's EMR.

The clinical assessment system 100 dynamically updates the trained LLM 102 with Sarah's new data and the provider's feedback, improving the accuracy of future insights. Using a RAG technique, the cognitive analysis module 116 retrieves the latest clinical research on prediabetic neuropathy and anxiety management from the healthcare knowledge database. The clinical assessment system 100 generates and delivers the clinical insights such as identified conditions, including potential prediabetic neuropathy and high-stress levels, prioritized for intervention. The clinical assessment system 100 also generates and delivers treatment recommendations such as tailored dietary adjustments, physical activity plans, and CBT sessions. The clinical assessment system 100 also generates and delivers interactive reports such as a summary of Sarah's glucose trends, anxiety triggers, and real-time symptom progression for clinician review. The clinical assessment system 100 also generates and delivers predictive assessments such as a projection of Sarah's glucose trends based on her current dietary patterns and physical activity levels. The clinical assessment system 100 also generates and delivers personalized treatment plans such as updated care recommendations synchronized with Sarah's EMR for continuity of care.

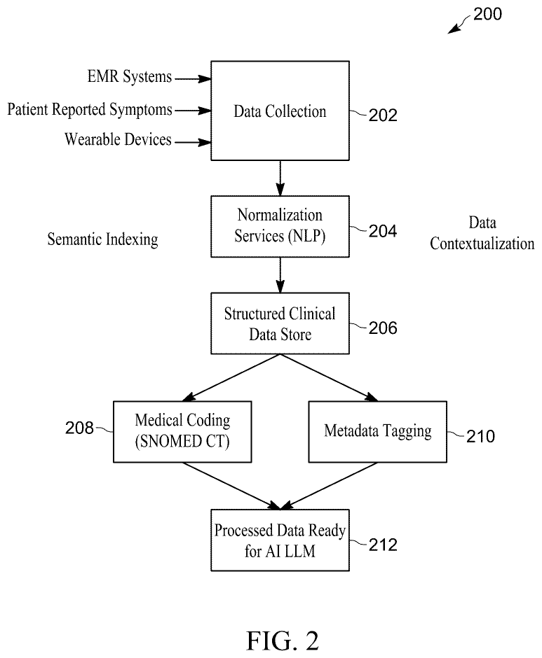

Referring now to FIG. 2, an example flow diagram of processing the patient health data, is illustrated, in accordance with an embodiment of the present disclosure. The flow diagram 200 demonstrates the operations involved in processing patient health data for subsequent analysis by the trained LLM 102.

The process begins at a data collection block 202, which receives patient health data from the input sources. These input sources may include the wearable devices 138 and the patient data repository 106, which may include patient-reported symptoms 140, input 142, and the EMR 144. The patient health data received may include, but not limited to, one or more of biometric data from the wearable devices 138, historical medical data 146, and patient-reported symptoms 140.

Thereafter, the received data is subsequently passed to a normalization services block 204, which employs NLP techniques to standardize the patient health data. The normalization services block 204 involves semantic indexing to ensure that the patient health data from the input sources is translated into a uniform format. The normalization process resolves inconsistencies in terminology, structure, and representation of the patient health data. For example, NLP may standardize patient-reported symptoms or wearable device metrics into a structured format compatible with the clinical assessment system 100. The normalized data is stored within a structured clinical data store 206, which acts as a central repository for organized and indexed patient health data. This structured format ensures that the patient health data is readily accessible for subsequent processing tasks. The structured clinical data store 206 enables data contextualization. The contextualized data from the structured clinical data store 206 is further processed through two parallel pathways: medical coding block 208 and metadata tagging block 210. The medical coding block 208 maps the structured data to standardized medical coding systems such as SNOMED CT or LOINC. This ensures interoperability across various healthcare systems and platforms, allowing consistent interpretation of clinical data. For instance, symptoms and diagnoses are encoded in a standardized format, which can be universally understood.

Concurrently, the metadata tagging block 210 augments the patient health data with metadata. These metadata may include temporal data (e.g., the timing of symptom onset), locational data (e.g., where the patient received care), and categorical data (e.g., type of medical intervention). For example, metadata may indicate a correlation between specific patient-reported symptoms and time of day. Both the coded data from the medical coding block 208 and the patient health data from the metadata tagging block 210 are combined to form a processed data 212, which is ready for analysis by the trained LLM 102. The processed data 212 serves as an input to the trained LLM 102.

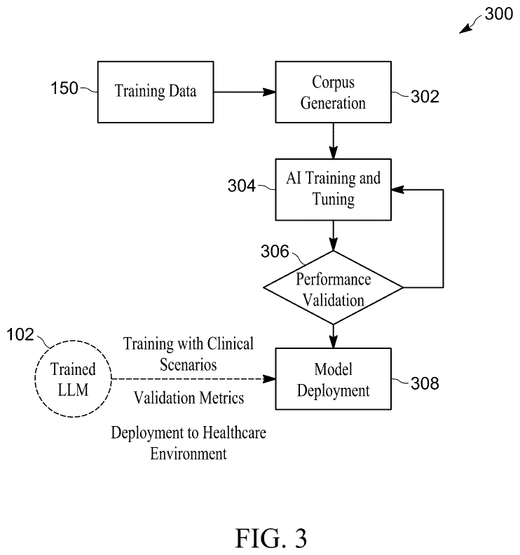

Referring now to FIG. 3, an example flow diagram of training and initialization of the trained LLM 102, is illustrated, in accordance with an embodiment of the present disclosure. The flow diagram 300 represents the structured approach to building, training, validating, and deploying the trained LLM 102 to operate as an analytical component of the clinical assessment system 100. The process begins with the training data 150, which forms the foundational knowledge base for training the LLM 102. The training data 150 may include, but is not limited to, a diverse range of healthcare-related data such as psychiatric research, historical patient data, treatment outcomes, and medical literature.

The training data 150 is fed into the corpus generation module 302, which preprocesses and structures the training data 150 into a training-ready format. The corpus generation module 302 performs tasks such as data cleaning, tokenization, and semantic tagging. The corpus generation module 302 ensures that the training data 150 is transformed into an optimized, structured corpus that captures the semantic and contextual degrees required for effective LLM training. The structured training data is then passed to an AI training and tuning module 304, where the initial training of the LLM 102 occurs. The tuning module 304 utilizes machine learning frameworks such as TensorFlow® or PyTorch® to train the LLM 102 on the training data 150. The training process involves adjusting the LLM 102 parameters through iterative learning cycles to optimize performance.

Once the initial training is complete, the trained LLM 102 undergoes performance validation module 306, which serves as an evaluation step. The performance validation module 306 assesses the trained LLM 102 against predefined validation metrics, including accuracy, recall, precision, and contextual understanding. This step may also include testing the trained LLM 102 with real-world clinical scenarios to gauge its effectiveness in generating clinical insights, diagnoses, and recommendations. If the trained LLM 102 fails to meet the performance thresholds, the training and tuning module 304 may be re-engaged for additional refinement.

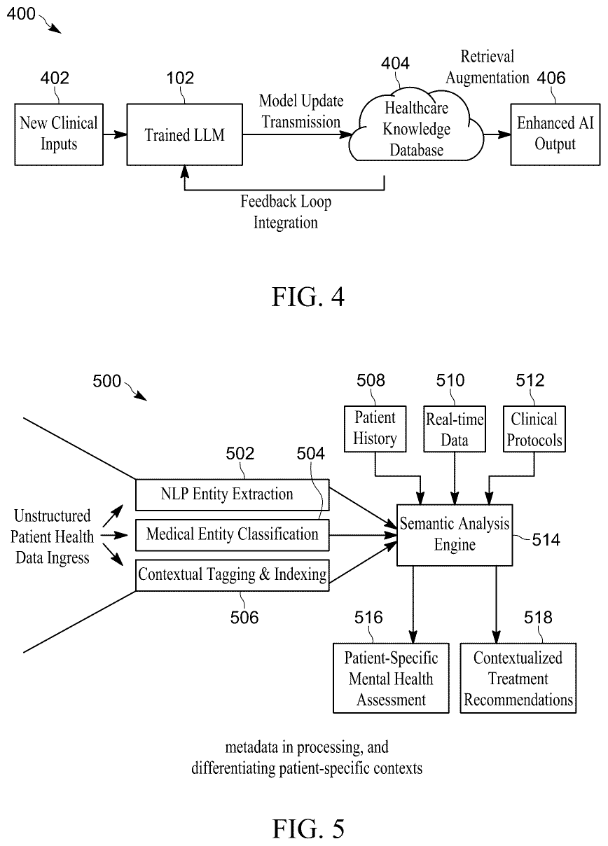

Following successful validation, the trained LLM 102 progresses to model deployment step 308. At this stage, the trained LLM 102 is prepared for integration into the clinical assessment system 100. The deployment process involves embedding the trained LLM 102 into the system architecture of the clinical assessment system 100, including integration with the clinical workflow engine 108, the patient interface 110, and the clinician interface 112. The model deployment step 308 ensures seamless operation of the trained LLM 102 in a live healthcare environment. The trained LLM 102 may be initialized within the healthcare environment. The trained LLM 102 undergoes additional training with clinical scenarios provided by real-world healthcare settings, as will be described in greater detail in FIG. 4.

Referring now to FIG. 4, an example flow diagram of dynamically updating the trained LLM 102, is illustrated, in accordance with an embodiment of the present disclosure. The flow diagram 400 depicts the iterative process by which the trained LLM 102 is dynamically updated with the patient health data.

New clinical inputs 402, which may include the patient health data, real-time updates from the wearable devices 138, the EMR 144, and clinician feedback from the workflow engine 108 may be received. These new clinical inputs 402 provide real-time patient health data for continuous refinement of the trained LLM 102. The new clinical inputs 402 are processed by a trained LLM 102, which serves as a component for analyzing and integrating the new clinical inputs 402. The trained LLM 102 utilizes advanced machine learning techniques, including contextual analysis, semantic understanding, and pattern recognition, to extract meaningful insights from the new clinical inputs 402. The trained LLM 102 also incorporates existing metadata and standardized coding systems (e.g., SNOMED CT, LOINC) to ensure interoperability and consistency in the analysis. The processed data are transmitted as a model update transmission to the healthcare knowledge database 404. The healthcare knowledge database 404 functions as a centralized repository of accumulated clinical knowledge, including prior training datasets, medical literature, and historical patient data. This healthcare knowledge database 404 is continuously updated through RAG techniques to enable the LLM to expand its contextual and semantic understanding dynamically.

The RAG techniques employed by the healthcare knowledge database 404 retrieve relevant data subsets from the healthcare knowledge database 404 to supplement the processing capabilities of the trained LLM 102. By doing so, the trained LLM 102 utilizes both historical knowledge and real-time updates (e.g., new clinical research studies, recent diagnostic guidelines, updated medication protocols, or real-time wearable device data) to enhance its predictive accuracy and contextual relevance. Based on the enriched knowledge from the healthcare knowledge database 404, the trained LLM 102 generates enhanced AI outputs 406. These outputs may include potential diagnoses, treatment recommendations, prioritized clinical conditions, predictive health assessments, and adaptive care plans tailored to individual patient needs. The enhanced AI outputs 406 are further validated and contextualized through a feedback loop integration. The feedback loop integration enables continuous improvement of the trained LLM 102. Feedback may be received from clinicians using the clinician interface 112 and/or patient responses using the patient interface 110.

Referring now to FIG. 5, an example flow diagram 500 of semantic analysis and context-aware processing is illustrated, in accordance with an embodiment of the present disclosure. FIG. 5 depicts the process by which unstructured clinical data is transformed into patient-specific mental health assessments and contextualized treatment recommendations through a series of semantic and contextual analysis steps. While the example of FIG. 5 includes details about mental health, physical health may also be addressed as well and/or assessed separately to mental health.

Unstructured patient health data may be input into the system. In some embodiments, the patient health data may include, but is not limited to, one or more of biometric data from the wearable devices 138, historical medical data 146, patient-reported symptoms 140, and the EMR 144. This unstructured patient health data is processed through multiple analytical stages to extract relevant medical information and provide clinical insights. The process may include NLP entity extraction 502, which utilizes NLP techniques to identify medical entities/data, such as symptoms, conditions, medications, and lab results, from unstructured clinical data. The extracted entities serve as elements for subsequent analyses.

The extracted entities are then categorized through medical entity classification 504, where the clinical assessment system 100 assigns standardized medical codes, such as SNOMED CT or LOINC, or the like, to the identified entities. This classification ensures interoperability and enables consistent interpretation across healthcare systems. In parallel, the clinical assessment system 100 performs contextual tagging and indexing 506 to enrich the patient health data with metadata. These metadata include, but are not limited to, temporal information (e.g., event timestamps), locational details (e.g., healthcare facility), and/or categorical classifications (e.g., patient demographics).

The outputs from the NLP entity extraction 502, the medical entity classification 504, and the contextual tagging and indexing 506 are transmitted to the semantic analysis engine 514, which integrates these components with additional data sources, including patient history 508, real-time data 510, and clinical protocols 512. The patient history 508 may include longitudinal medical records, such as past diagnoses and treatments. The real-time data 510 includes dynamic inputs, such as wearable device readings and recent lab results. The clinical protocols 512 encompass evidence-based guidelines and best medical practices.

The semantic analysis engine 514 utilizes machine learning models, including the trained LLM 102, to perform advanced semantic and contextual processing. By synthesizing data from multiple sources, the semantic analysis engine 514 may generate one or more primary outputs, for example, patient-specific mental health assessments 516 (and/or physical health assessments) and contextualized treatment recommendations 518. The patient-specific mental health assessments 516 (and/or physical health assessments) provide a detailed understanding of the current mental health status (or physical health status) of the patient, including prioritized conditions, potential risk factors, and symptom trajectories. These assessments are tailored to the individual clinical context. The contextualized treatment recommendations 518 offer clinical insights for healthcare providers, such as personalized treatment plans, medication adjustments, and lifestyle intervention strategies. These recommendations are aligned with the patient's unique clinical profile and adhere to established medical guidelines.

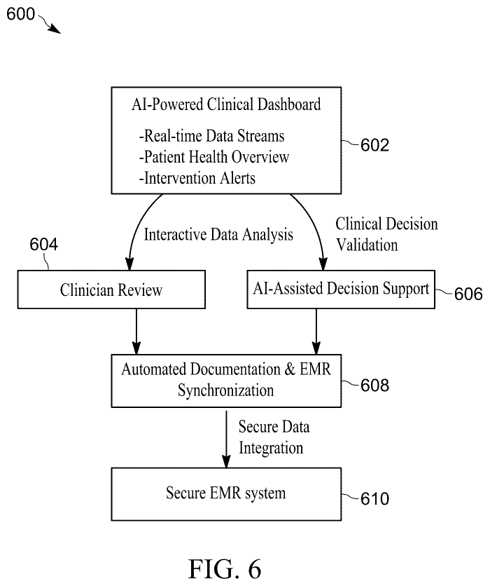

Referring now to FIG. 6, an example flow diagram of clinical workflow and decision support is illustrated, in accordance with an embodiment of the present disclosure. FIG. 6 depicts the interconnected components and processes involved in enabling real-time clinical decision-making and seamless integration with EMR 144.

The clinician dashboard 602 may be integrated into the clinician interface 112, which provides clinicians with a consolidated view of real-time data streams, a comprehensive patient health overview, and intervention alerts. This clinician dashboard 602 acts as the primary interface for interacting with the clinical insights and serves as a decision-making hub. The clinician dashboard 602 provides clinicians with clinical insights in a user-friendly format. The clinician dashboard 602 enables interactive data analysis and clinical decision validation through one or more pathways: the clinician review pathway 604 and AI-assisted decision support pathway 606. In the clinician review pathway 604, healthcare providers manually evaluate the clinical insights, utilizing their expertise to validate or modify the recommendations. This clinician review pathway 604 ensures that clinical decisions align with established medical practices and patient-specific contexts. Alternatively, the AI-assisted decision support pathway 606 utilizes advanced algorithms within the clinical workflow engine to autonomously suggest potential treatment plans, identify critical risk factors, and flag inconsistencies in the patient health data. This pathway streamlines the decision-making process, thereby allowing clinicians to focus on high-priority cases and improving efficiency in high-volume clinical settings. Once decisions are validated or refined through either pathway, the flow diagram 600 proceeds to automated documentation and EMR synchronization 608. The EMR synchronization 608 involves the automatic generation of clinical notes, treatment plans, and diagnostic summaries based on the finalized decisions. The documentation is formatted to comply with standards such as FHIR (Fast Healthcare Interoperability Resources) to ensure compatibility with diverse EMR systems. The documentation generated at the EMR synchronization 608 is securely integrated with the secure EMR system 610 through a robust data integration framework. This framework employs encryption protocols and role-based access controls to maintain the confidentiality and integrity of patient health data.

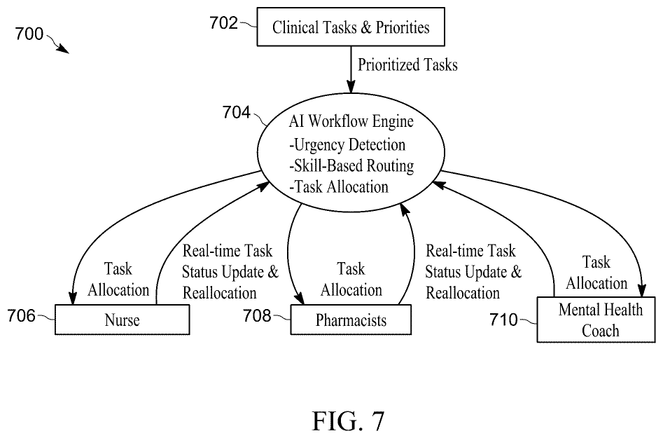

Referring now to FIG. 7, an example flow diagram 700 of task allocation and workflow management is illustrated, in accordance with an embodiment of the present disclosure. FIG. 7 depicts the operational framework of a task allocation system integrated with an AI workflow engine 704 to ensure optimized resource utilization and efficient management of clinical workflows. The clinical tasks and priorities module 702 receives a list of tasks based on current clinical demands, patient care priorities, and organizational objectives. The priorities module 702 organizes the tasks and assigns priority levels, ensuring that high-urgency tasks are flagged for immediate attention. For example, tasks such as medication review or patient monitoring with critical conditions may be prioritized over routine follow-ups.

The prioritized tasks are transmitted to the AI workflow engine 704, which is equipped with capabilities for urgency detection, skill-based routing, and task allocation. The urgency detection component evaluates the criticality of each task using real-time patient data and predefined clinical protocols. The skill-based routing functionality maps tasks to appropriate healthcare providers based on their expertise, availability, and workload. The task allocation mechanism ensures that each task is dynamically assigned to suitable team member. The task allocation process is illustrated through three representative roles in FIG. 7, nurse 706, pharmacist 708, and mental health coach 710. The AI workflow engine 704 allocates tasks to these roles based on specific criteria. For instance, medication reconciliation tasks may be routed to the pharmacist 708, while patient counseling activities could be allocated to the mental health coach 710. Similarly, tasks such as vital sign monitoring may be assigned to the nurse 706.

Once tasks are assigned, the clinical assessment system 100 enables real-time task status updates and reallocation. This feature ensures continuous monitoring of task progress and allows the AI workflow engine 704 to dynamically reallocate tasks in response to delays, resource availability changes, or unforeseen circumstances. For example, if the nurse 706 encounters an unexpected workload, the system may reassign non-critical tasks to other team members, such as the mental health coach 710.

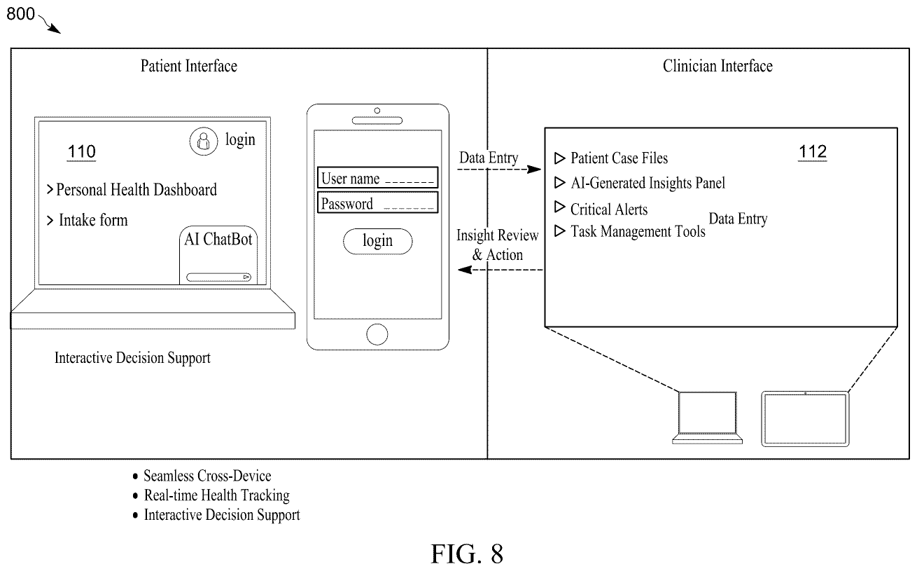

Referring now to FIG. 8, an example dual-panel diagram depicting the patient interface 110 and the clinician interface 112, is illustrated, in accordance with an embodiment of the present disclosure. Left side of the dual-panel diagram 800 represents the patient interface 110, which is designed for direct interaction with patients to enable data input, real-time health tracking, and interactive decision support. The patient interface 110 can be accessed through multiple devices, including desktop computers, mobile phones, and tablets. The patient interface 110 provides several functionalities such as a Personal Health Dashboard which enables patients to view their health metrics, treatment progress, and personalized insights generated by the trained LLM 102. The patient interface 110 further provides an intake form in which patients can input symptoms, medical history, and lifestyle information, which is processed and analyzed by the cognitive analysis module 116 of the clinical workflow engine 108. The patient interface 110 further provides an AI Chatbot which is embedded within the patient interface 110, the AI chatbot provides a conversational interface for patients to ask questions, receive guidance, and clarify medical instructions.

The right side of the dual-panel diagram 800 represents the clinician interface 112, which provides healthcare providers with tools for reviewing and managing patient health data, as well as LLM-generated clinical insights. The clinician interface 112 offers features such as patient case files in which clinicians can access the patient health data, including the historical medical data 146, real-time updates from wearable devices 138, and the EMR 144. The clinician interface 112 further offers an AI-generated insights panel that displays clinical insights, such as potential diagnoses, prioritized conditions, and treatment recommendations, generated by the insight generator 122. The clinician interface 112 further offers critical alerts for urgent matters, such as potential medication contraindications or significant health deterioration. The clinician interface 112 further offers task management tools that enable workflow management by allowing clinicians to assign tasks, track progress, and collaborate with other healthcare providers.

Data flow between the patient interface 110 and the clinician interface 112 is bi-directional. Patients enter data using intake forms or the AI chatbot, which is processed by the clinical workflow engine 108. The resulting clinical insights and updates are transmitted to the clinician interface 112 for review and validation. Conversely, clinicians can update care plans or recommendations, which are communicated back to the patient interface 110 for patient action or acknowledgment.

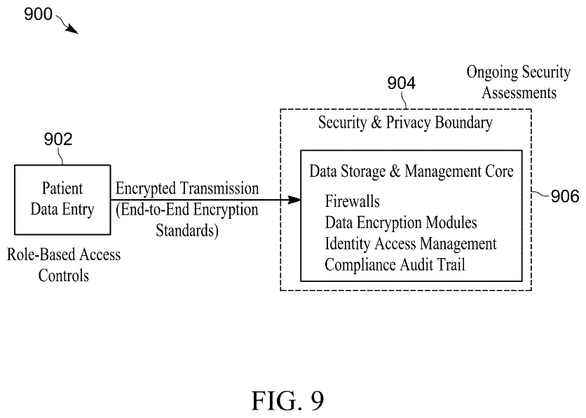

Referring now to FIG. 9, an example schematic diagram depicting encryption of the patient health data, is disclosed, in accordance with an embodiment of the present disclosure. The schematic diagram 900 depicts the secure handling, storage, and management of the patient health data within the clinical assessment system 100. The process begins with the patient data entry module 902, which represents the point at which patient health data, such as one or more of biometric data from the wearable devices 138, historical medical data 146, and patient-reported symptoms 140, is entered into the clinical assessment system 100. This patient health data may be entered at the patient interface 110 or other integrated data collection devices, such as the wearable devices 138 or the EMR 144. To ensure role-based access control, the clinical assessment system 100 enforces strict authentication and authorization protocols, thereby preventing unauthorized access to sensitive information. Data entered through the patient data entry module 902 is transmitted securely using end-to-end encryption standards, thereby ensuring that data integrity and confidentiality are maintained during transmission.

The encrypted data is then directed into the Security and Privacy Boundary 904, which defines the protected perimeter of the data storage and management infrastructure of the clinical assessment system 100. The Security and Privacy Boundary 904 employs multiple layers of security controls to ensure robust protection against unauthorized access or breaches. Components within the Security and Privacy Boundary 904 may include firewalls, data encryption modules, identity access management (IAM), and compliance audit trail. In some embodiments, the firewalls filter incoming and outgoing network traffic, blocking unauthorized access and preventing potential threats. In some embodiments, the data encryption modules ensure that data remains encrypted both in transit and at rest, utilizing encryption protocols that comply with healthcare standards such as HIPAA. In some embodiments, the IAM enforces role-based access control, allowing authorized users to access specific datasets or functionalities within the clinical assessment system. In some embodiments, the compliance audit trail logs access attempts, modifications, and system interactions, ensuring traceability and accountability. It supports compliance with regulatory standards, including HIPAA and GDPR.

Within the data storage and management core 906, the patient health data is securely stored and managed. This data storage and management core 906 ensures that the patient health data is structured, indexed, and accessible for clinical analysis and AI/ML model training (e.g., the trained LLM 102). Additionally, ongoing security assessments are performed, which include vulnerability scans, penetration testing, and real-time monitoring to identify and mitigate emerging threats.

In some embodiments, the system is designed to provide the highest levels of security, compliance, and data protection, ensuring that patient information remains confidential and secure. The system adheres to regulatory standards such as HIPAA (Health Insurance Portability and Accountability Act) and HITECH (Health Information Technology for Economic and Clinical Health Act), guaranteeing that Protected Health Information (PHI) is handled securely. Furthermore, in the event that system 100 is utilized in a country outside of the United States, other protocols, rules, laws, and/or regulatory standards can be employed or otherwise accessed. The system maintains confidentiality, integrity, and availability through measures such as role-based access control (RBAC), automated validation processes, and disaster recovery strategies, ensuring patient data remains protected even in the event of system failures or cyberattacks. Additionally, since the system operates on one or more a cloud servers (e.g., healthcare-specific cloud server(s)), it includes a Business Associate Agreement (BAA) that further ensures compliance with HIPAA standards and/or other predetermined standards, protocols, rules, or laws.

To protect data from unauthorized access, encryption mechanisms are in place, securing information both at rest and in transit using encryption (e.g., AES-256 or the like). Furthermore, the system implements end-to-end encryption, allowing sensitive information like psychiatric reports and medication plans to be encrypted based on access privileges. The system also integrates Advanced Threat Protection (ATP) across services that such as one or more chat services, file sharing services, file storage services, and analytics tools, etc., providing continuous monitoring for security threats like malware and ransomware. Any detected threats generate real-time alerts to system administrators for immediate action.

A Role-Based Access Control (RBAC) system ensures that users can access data relevant to their specific roles. This dynamic system automatically adjusts permissions as user roles change, minimizing unauthorized access. Granular access controls further restrict sensitive fields, such as psychiatric care plans, to authorized personnel like Lead Psychiatrists or Care Managers. Additionally, audit trails track all user activity, including data access and modifications, with security analytics software to provide a centralized monitoring dashboard for detecting anomalies and unauthorized access attempts.

To maintain accountability and ensure transparency, the system keeps comprehensive audit logs of all activities, from patient record updates to workflow changes. These logs are integrated with security analytics software, allowing administrators to track access patterns and potential security breaches in real-time. Automated compliance audits are also conducted using system 100, which generates reports identifying data access violations or unusual activity. If any compliance breaches are detected, the system triggers automated alerts for immediate resolution.

The system also features real-time security monitoring, which continuously scans for vulnerabilities and configuration risks. With Security Information and Event Management (SIEM) capabilities, threats are proactively identified, alerts are generated, and security incidents are swiftly addressed. Additionally, Advanced Threat Protection (ATP) is deployed across all services provided by system 100, ensuring that malware and ransomware attacks are flagged before they can cause damage.

For data governance and privacy, the system leverages data governing software to classify and manage patient data according to regulatory standards. This allows for the enforcement of Data Loss Prevention (DLP) policies, preventing unauthorized sharing of sensitive patient information across other services accessible to system 100. Furthermore, the system complies with regional data residency laws, such as General Data Protection Regulation (GDPR) and California Consumer Privacy Act (CCPA) (or the like), ensuring that patient data is stored within legally approved regions. The cloud computing platforms used by system 100 may provide regional services to allow data to be stored in compliance with local regulations, and regular audits ensure continued adherence to these legal requirements.

The system provides a robust framework for security, compliance, and data protection, integrating advanced encryption, real-time threat monitoring, role-based access control, and automated compliance audits. By leveraging the described security tools, the system 100 ensures data integrity, confidentiality, and availability, while proactively defending against evolving cyber threats.

The Compliance Audits and Reporting module in the system is designed to ensure that healthcare organizations comply with critical regulatory standards such as HIPAA (Health Insurance Portability and Accountability Act) and HITECH (Health Information Technology for Economic and Clinical Health Act), along with internal compliance policies. This module plays a role in maintaining the security, privacy, and integrity of patient data while ensuring operational transparency and accountability within care teams.

To achieve this, the system utilizes automated tools that continuously monitor and track user activities within the platform. It generates detailed audit logs that document actions such as patient record access, data modifications, and task completions. These logs help administrators review system usage, detect potential security breaches, and ensure that staff members adhere to compliance guidelines.

Additionally, the module includes automated policy violation reporting, which identifies unauthorized access, irregular activity, or deviations from established compliance protocols. When a potential violation is detected, the system can trigger alerts, notify administrators, and provide corrective action recommendations.

By leveraging real-time monitoring, automated reporting, and robust audit capabilities, the Compliance Audits and Reporting module enables healthcare organizations to proactively manage compliance risks, streamline regulatory audits, and protect sensitive patient information. This ensures that care teams operate within legal and ethical standards while maintaining trust and accountability in patient care operations.

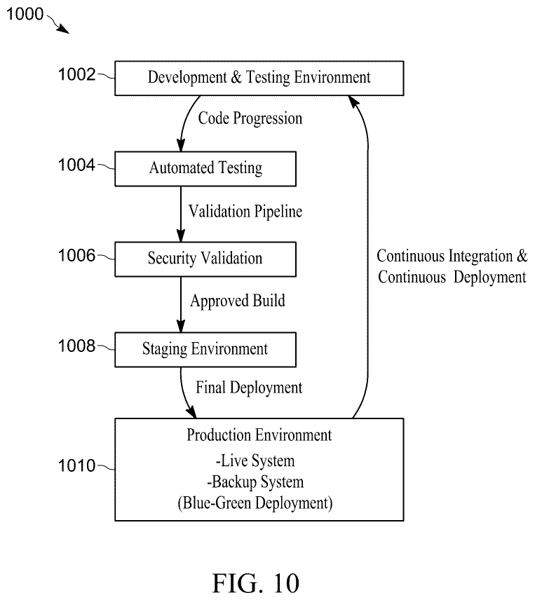

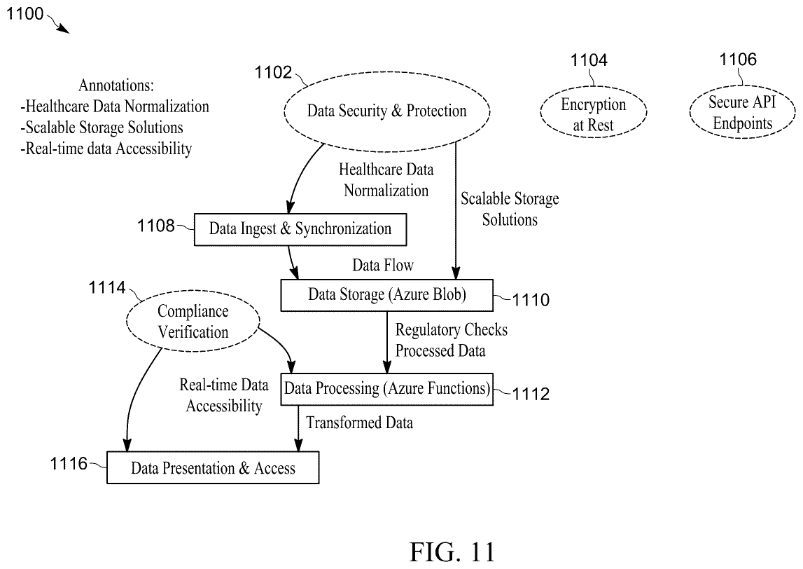

Referring now to FIG. 10, an example flow diagram of testing, deployment, and updates of the clinical assessment system 100 is illustrated, in accordance with an embodiment of the present disclosure. This flow diagram 1000 depicts the systematic approach for continuous integration (CI) and continuous deployment (CD) of the clinical assessment system 100.

The initial code progression of the clinical assessment system 100 occurs in the development and testing environment 1002. The testing environment 1002 serves as the workspace for developing new features, fixing bugs, and implementing updates to the clinical assessment system 100. Once changes are made, the initial code enters the testing pipeline for further validation. The next phase involves automated testing 1004, which includes running a series of automated test cases designed to identify bugs, verify functionality, and ensure code quality. This step may include unit tests, integration tests, and regression tests. Automated testing ensures that new code does not disrupt existing system functionalities. Following successful automated testing, the code proceeds to the security validation phase 1006, where it undergoes rigorous checks to identify and mitigate potential vulnerabilities. This may include static and dynamic application security testing, penetration testing, and compliance verification with healthcare industry standards such as HIPAA. A code that passes all security checks is approved for further deployment.

After security validation, the code enters the staging environment 1008, which mimics the production environment. This staging phase allows for live testing of the approved build in a controlled environment to identify any issues that might arise in a real-world setting. This block helps ensure that the system can handle expected user loads and provides a seamless user experience.

Upon successful validation in the staging environment, the system proceeds to the production environment 1010 for final deployment. The production environment employs a blue-green deployment strategy, where identical or substantially identical environments (e.g., live system and backup system) are maintained. During deployment, updates are first applied to the backup system (green environment) while the live system (blue environment) continues to operate without interruptions. Once the updates are validated in the backup system, traffic is rerouted to it, effectively making it the new live system. This approach minimizes downtime and ensures rollback capability in case of deployment issues.

Referring now to FIG. 11, an example architectural diagram of cloud-based data management, is illustrated, in accordance with an embodiment of the present disclosure. The architectural diagram 1100 depicts the multi-layered approach employed for the secure, scalable, and efficient management of healthcare data within the clinical assessment system 100.

The data management workflow includes data ingest and synchronization 1108, which is responsible for aggregating data from various input sources such as the wearable devices 138, the patient data repository 106 that includes patient-reported symptoms 140, input 142, and the EMR 144. Data security and protection 1102 ensures that patient health data operations adhere to stringent security protocols. This includes the implementation of encryption at rest 1104, which safeguards stored data using encryption technologies, and secure API endpoints 1106, which protect data during transmission to and from external systems. Once ingested, the data is routed to data storage 1110, which utilizes cloud storage solutions such as Azure Blob for scalable and secure storage. The storage architecture is optimized to handle large volumes of healthcare data efficiently while maintaining redundancy for data integrity. The next layer, data processing 1112, employs serverless computing capabilities such as Azure Functions to perform complex transformations and analyses on the raw data. This block ensures that the processed data meets the system's requirements for subsequent stages, such as clinical insights generation. The architecture incorporates a compliance verification 1114 module, which performs regulatory checks to ensure that all data operations are in compliance with healthcare standards and regulations such as HIPAA and GDPR. This module also audits the data handling processes to maintain accountability and transparency. The processed and verified data is then made available through the data presentation and access 1116 layer, which delivers transformed data to the system's various interfaces, including the patient interface 110 and clinician interface 112. This layer ensures real-time data accessibility and supports seamless integration with external systems.

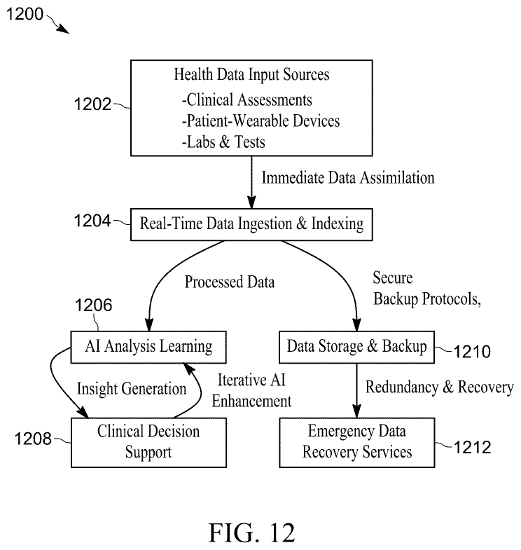

Referring now to FIG. 12, an example flow diagram of real-time data handling within the clinical assessment system 100, is illustrated, in accordance with an embodiment of the present disclosure. The flow diagram 1200 demonstrates the systematic processing of patient health data. The process includes health data input sources 1202, which include the wearable devices 138, and the patient data repository 106 which includes patient-reported symptoms 140, input 142, and the EMR 144.

The patient health data is passed to the real-time data ingestion and indexing module 1204, which organizes and indexes the patient health data for efficient processing. This block ensures that the patient health data is accessible and ready for use by various system components. The indexed data is subsequently bifurcated into one or more processing streams, AI analysis and learning module 1206 and data storage and backup module 1210. The AI analysis and learning module 1206 processes the indexed data to generate clinical insights through iterative AI enhancement. This AI analysis and learning module 1206 includes capabilities for real-time updates and refinement of the trained LLM 102. The insights generated by the learning module 1206 are directed to the clinical decision support module 1208, which provides healthcare providers with actionable recommendations and diagnoses to optimize patient care. Simultaneously, the processed data is stored securely in the data storage & backup module 1210. This 1210 module ensures that the data is maintained with redundancy protocols. Additionally, this data storage and backup module 1210 enables secure backup protocols to protect the data and enable seamless recovery in case of system failures. The emergency data recovery services module 1212 works in conjunction with the data storage and backup systems. The emergency data recovery services module 1212 ensures that critical data can be retrieved quickly during emergencies, minimizing downtime and ensuring the continuity of patient care.

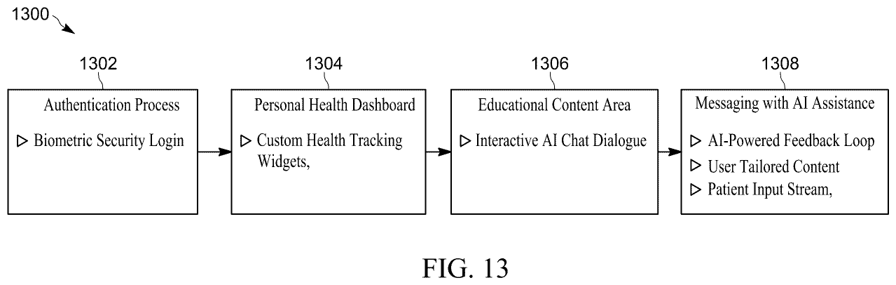

Referring now to FIG. 13, an example schematic diagram 1300 of the patient interface 110, is illustrated, in accordance with an embodiment of the present disclosure. The patient interface 110 provides a user-centric platform designed to enable seamless interaction between patients and the healthcare system while offering personalized features to enhance patient engagement and care management. The process includes the authentication process 1302, which ensures secure access to the patient interface 110. The authentication process may involve a biometric security login, such as fingerprint scanning, facial recognition, or other biometric authentication methods. Upon successful authentication, the patient interface 110 transitions to the personal health dashboard 1304, which serves as the primary hub for the patient. The personal health dashboard 1304 includes custom health tracking widgets, allowing patients to monitor vital statistics, medication schedules, and other health parameters. These widgets can be tailored to the specific needs and preferences of individual patients, enhancing usability and personalization.

The patient interface 110 further incorporates an educational content area 1306, designed to empower patients with knowledge about their conditions and care plans. This area includes an interactive AI chat dialogue, where patients can ask questions, receive real-time answers, and engage with AI-powered educational resources. This feature promotes patient awareness and enables informed decision-making regarding their healthcare. The final component of the patient interface is messaging with AI assistance 1308, which enables seamless communication between patients and the system.

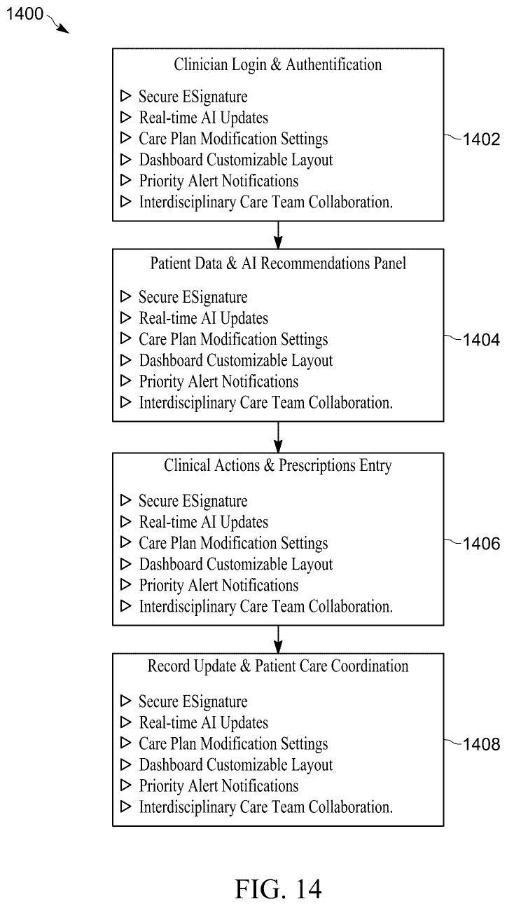

Referring now to FIG. 14, an example schematic diagram 1400 of the clinician interface 112, is illustrated, in accordance with an embodiment of the present disclosure. The clinician interface 112 is designed to enable efficient clinical workflows, enabling healthcare providers to manage patient data, review AI-generated recommendations, and perform critical actions seamlessly.

The clinician interface 112 includes a clinician login and authentication module 1402, which ensures secure access to the clinical assessment system 100. The clinician login and authentication module 1402 includes features such as “Secure E-Signature” for authentication and authorization of the clinician, “Real-Time AI Updates” for keeping clinicians informed about patient statuses, and “Care Plan Modification Settings” for customizing treatment plans. Additionally, the clinician login and authentication module 1402 supports a “Dashboard Customizable Layout”, allowing clinicians to tailor the clinician interface 112 to their specific workflow needs. Features such as “Priority Alert Notifications” highlight critical cases requiring immediate attention, and “Interdisciplinary Care Team Collaboration” enables communication and coordination among different healthcare providers.

After logging in, clinicians access the patient health data and AI recommendations panel 1404, which consolidates patient information and AI-driven clinical insights. This panel includes tools for Secure E-Signature, enabling clinicians to approve AI-generated recommendations securely. Real-Time AI Updates provide instant visibility into evolving patient data, while care plan modification settings allow adjustments to treatment protocols. The clinical actions and prescriptions entry module 1406 enables clinicians to input treatment decisions, orders, and prescriptions. This clinical actions and prescriptions entry module 1406 also incorporates secure E-signature for validation, real-time AI updates for contextual guidance, and care plan modification settings for dynamic changes. The customizable dashboard layout enhances usability, and notifications and collaboration tools ensure cohesive care delivery across teams. The record update and patient care coordination module 1408 enables updating patient records and coordinating care activities. The record update and patient care coordination module 1408 integrates secure E-signature functionality, ensuring compliance and security, along with real-time AI updates for keeping records current. Clinicians can utilize care plan modification settings and a dashboard customizable layout to manage patient data effectively. Priority alert notifications ensure timely interventions, while interdisciplinary care team collaboration supports patient care planning.

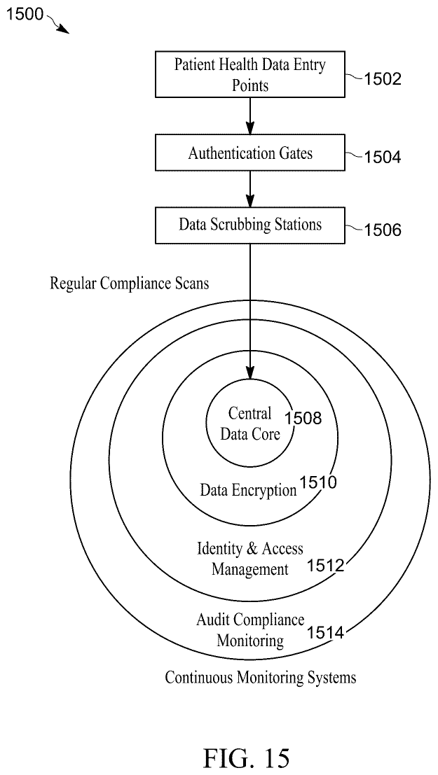

Referring now to FIG. 15, an example flow diagram 1500 of data security and privacy protocol within the clinical assessment system 100, is illustrated, in accordance with an embodiment of the present disclosure. The protocol ensures comprehensive protection for sensitive data through a multi-layered approach, encompassing data entry, authentication, and core-level encryption with compliance monitoring.

The protocol includes user data entry points 1502, where patient health data is collected through designated interfaces such as the patient interface 110, the clinician interface 112, or input sources such as the wearable devices 138 and the patient data repository 106 that includes the patient-reported symptoms 140, input(s) 142, and EMR 144. The input sources may also include the historical medical data 146, and DSM data sources 148. These entry points are designed to handle various forms of input, including structured data, free text, and multimedia files. The patient health data collected at these points undergoes preliminary checks for validity and completeness. The collected patient health data proceeds to authentication gates 1504, which verify the credentials of patients accessing the clinical assessment system 100. This layer includes multifactor authentication (MFA), biometric verification, and/or secure token-based access to prevent unauthorized entry. The authentication gates ensure that verified personnel or systems can proceed further into the workflow, maintaining data integrity and restricting unauthorized access.

After authentication, the patient health data is directed to data scrubbing station 1506, where it is cleansed and normalized. These stations remove redundant, erroneous, or irrelevant information and apply data transformation techniques to ensure consistency and standardization. In some embodiments, the data scrubbing stations utilize NLP algorithms to extract meaningful entities from unstructured text and semantic indexing techniques to align the data with predefined schemas. Following data scrubbing, patient health data enters the central data core 1508, which acts as a repository for securely storing and processing data. The core employs advanced data encryption techniques, depicted as data encryption 1510, to ensure that stored data remains confidential and protected from unauthorized access. Encryption is performed using industry-standard protocols such as AES-256 to safeguard the information both at rest and during transit.

Surrounding the central data core is an identity and access management layer 1512, which governs permissions and access controls. This layer ensures that authorized users and systems can retrieve or modify data. Role-based access controls (RBAC) and dynamic policy enforcement are implemented to restrict access based on user roles and the sensitivity of the data. An outer layer of the protocol includes audit compliance monitoring 1514, which conducts regular compliance scans and audits to ensure adherence to regulatory standards, such as HIPAA, GDPR, or FHIR guidelines. This layer enables real-time monitoring of system activities, generating logs for anomaly detection and forensic investigations in case of a breach. The framework may be supported by continuous monitoring systems, which ensure the protocol remains robust and adaptable to emerging threats. These systems utilize machine learning models to detect and mitigate potential vulnerabilities dynamically.

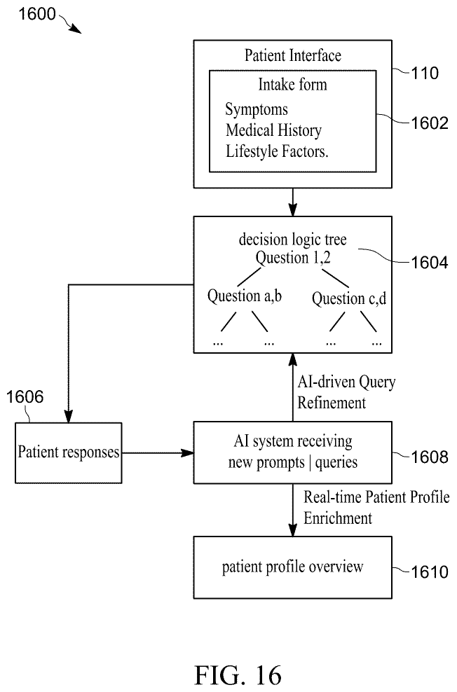

Referring now to FIG. 16, an example flow diagram 1600 of interactive prompting and data capture process during patient intake, is illustrated, in accordance with an embodiment of the present disclosure. This process utilizes the patient interface 110 and advanced AI-driven mechanisms to collect, refine, and analyze patient data, ensuring a comprehensive and personalized intake experience.

The process begins at the patient interface 110, which includes an intake form 1602. The intake form 1602 captures various data points such as patient-reported symptoms, medical history, and lifestyle factors provided by the patient. The patient interface 110 may be presented in multiple formats, including web-based forms, mobile applications, or voice-enabled systems, offering flexibility in patient interaction. The captured patient health data is processed by a decision logic tree 1604, which operates as a branching algorithm to guide the interaction. The decision logic tree presents questions (e.g., “Question 1, 2”) that branch into more specific queries (“Question a, b” and “Question c, d”) depending on the patient's input. This mechanism ensures that data collection is both comprehensive and relevant, dynamically tailoring the flow of questions to the patient's specific condition or context.

Based on the patient health data, patient responses 1606 are transmitted to an AI-driven system 1608 for refinement. The AI-driven system 1608, receiving new prompts and queries, utilizes the trained LLM 102 to analyze patient responses and generate additional prompts as needed. The AI-driven system 1608 employs advanced algorithms to refine the line of questioning to ensure that relevant information is captured in real-time. This iterative refinement may include semantic analysis and contextual understanding of patient-provided data. The responses and refined prompts are used for real-time patient profile enrichment 1610. The AI system consolidates and processes the collected data to create a comprehensive patient profile. This profile includes detailed insights into the patient's condition, potential risk factors, and personalized recommendations for subsequent clinical evaluation. The enriched profile is made accessible to healthcare providers using the clinician interface 112 for further review and decision-making.

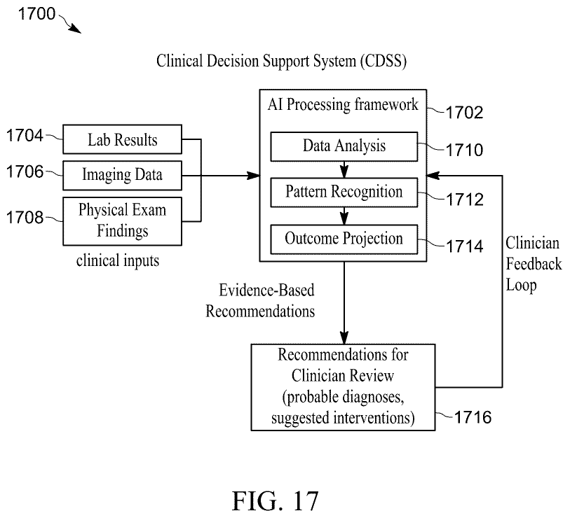

Referring now to FIG. 17, an example flow diagram 1700 of analyzing clinical data and generating treatment recommendations within the clinical assessment system 100, is illustrated, in accordance with an embodiment of the present disclosure. The clinical assessment system 100 integrates multiple data sources, applies advanced AI-based processing, and provides actionable recommendations for clinician review.

The process includes the input of clinical data, which includes lab results 1704, imaging data 1706, and physical exam findings 1708. These clinical inputs represent patient data from various diagnostic and examination procedures. The system supports diverse input formats, including structured, semi-structured, and unstructured data, ensuring compatibility with various healthcare systems. The clinical inputs are processed by the AI processing framework 1702, which serves as the computational engine of the clinical assessment system 100. This framework includes one or more sub-modules, for example, data analysis module 1710, pattern recognition module 1712, and/or outcome projection module 1714. The data analysis module 1710 preprocesses and contextualizes the clinical data, applying NLP, statistical methods, and semantic indexing to extract meaningful insights. The pattern recognition module 1712 utilizes machine learning algorithms to identify trends, anomalies, or correlations in the data, enabling the system to uncover hidden patterns that may inform clinical decision-making. The outcome projection module 1714 utilizes predictive analytics and probabilistic modeling to forecast potential outcomes based on the patient's clinical profile, considering historical data, known treatment pathways, and relevant medical literature.

Once processed, the AI processing framework 1702 generates evidence-based recommendations that are forwarded to the recommendations module 1716 for clinician review. This module consolidates probable diagnoses, suggested interventions, and other relevant clinical actions into a structured output. The recommendations are presented to clinicians in an interpretable format through the clinician interface 112, enabling validation, modification, or acceptance. The clinical assessment system 100 includes a clinician feedback loop which allows healthcare providers to input their decisions and feedback into the system. This feedback is transmitted back to the AI processing framework 1702, where it is used to refine the pattern recognition and outcome projection processes, ensuring continuous improvement and learning over time.

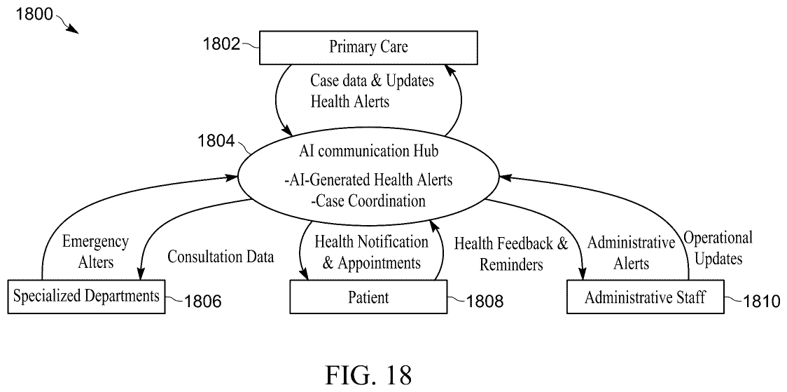

Referring now to FIG. 18, an example flow diagram 1800 of communication flow and notification within the clinical assessment system 100, is disclosed, in accordance with an embodiment of the present disclosure. The system 100 enables real-time communication, health alerts, and/or coordination between various stakeholders, enhancing patient care and operational efficiency. The central component of the system 100 may include an AI communication hub 1804, which integrates various data sources and manages notifications, case updates, and health alerts. The AI communication hub 1804 utilizes advanced algorithms, which may include, but are not limited to, NLP algorithms for interpreting unstructured patient inputs, decision-tree algorithms for prioritizing notifications based on urgency, and machine learning algorithms for predictive analytics. These algorithms may work collaboratively to ensure seamless communication and efficient case coordination.

The communication flow originates from a primary care 1802, which provides case data, updates, and health alerts to the AI communication hub 1804. The AI communication hub 1804 may act as a primary point for initiating patient health monitoring and care coordination. Information from primary care is disseminated to relevant stakeholders through the hub. The communication flow proceeds to specialized departments 1806 which contribute specialized consultation data and receive emergency alerts from the AI communication hub 1804. This interaction ensures that critical patient cases are escalated and addressed promptly. Furthermore, patients 1808 receive health notifications, appointment reminders, and feedback through the AI communication hub 1804. Additionally, administrative staff 1810 interacts with the clinical assessment system 100 by receiving operational updates and administrative alerts. The AI communication hub 1804 processes incoming data streams from all these entities, using AI algorithms (e.g., NLP algorithms for interpreting unstructured patient inputs, decision-tree algorithms for prioritizing notifications based on urgency, and machine learning algorithms for predictive analytics) to prioritize and route information efficiently. Health alerts and case updates are dynamically generated based on real-time patient conditions, operational changes, or new consultation inputs.

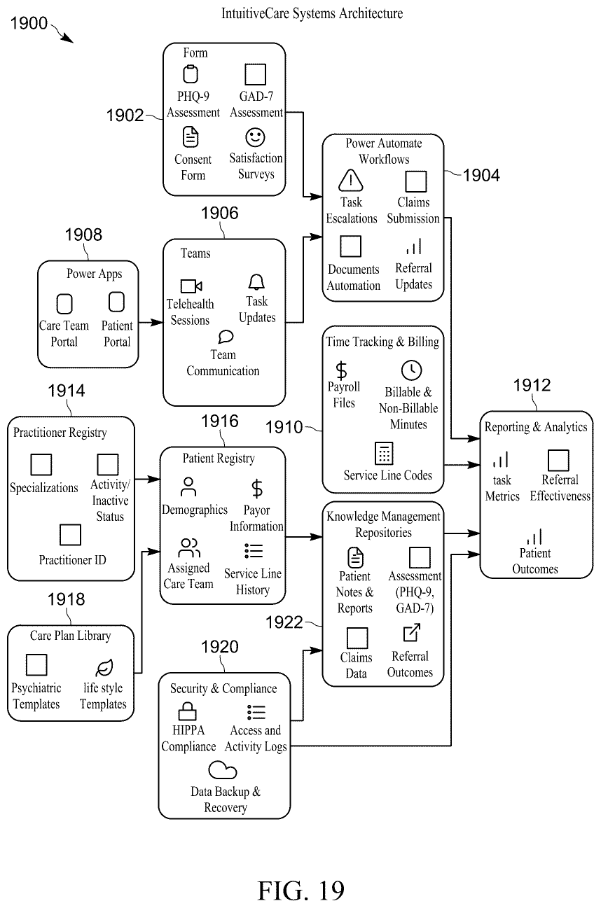

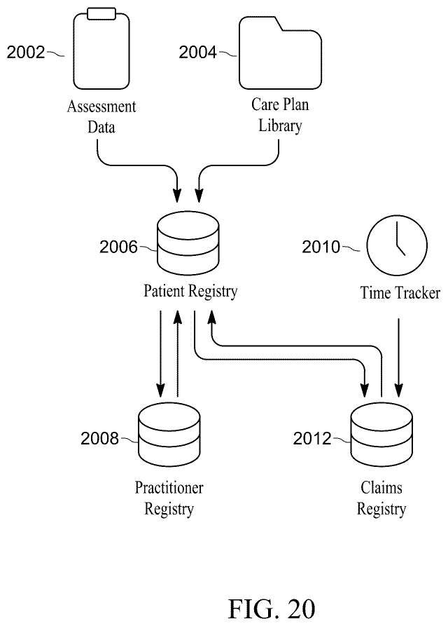

Referring now to FIG. 19, an example architectural diagram 1900 depicting patient-centered care delivery, is illustrated, in accordance with an embodiment of the present disclosure. The architecture integrates various modules and workflows to enable efficient care coordination, secure data management, and actionable analytics, ensuring holistic patient care. The process includes form submissions 1902, which include assessments, consent forms, and surveys capturing essential patient data. These data flow into subsequent workflows for further processing and integration. Further, workflows and automation 1904 streamline tasks such as escalations, claims submissions, and document generation. Automated processes ensure timely notifications and updates across the system, enhancing operational efficiency. Further, communication modules 1906 enables real-time interactions among care teams. These modules support telehealth sessions, task updates, and secure messaging, enabling seamless collaboration. In some embodiments, the system 100 supports synchronous and asynchronous: telehealth, remote monitoring, and multi-tenant deployment with configurable privacy, workflow, and interoperability per tenant or per jurisdiction. Further, portals 1908, including care team and patient interfaces, serve as access points for interacting with the system. The portals provide functionalities such as monitoring patient progress, scheduling tasks, and retrieving care plans. The time tracking and billing module 1910 captures billable and non-billable minutes associated with care delivery. The time tracking and billing module 1910 (e.g., time tracking and billing system 156 of FIG. 1A) integrates with service line codes to support automated claims generation and financial reconciliation.

Additionally, a reporting and analytics module 1912 aggregate data from multiple modules to provide insights into task metrics, referral effectiveness, and patient outcomes. This module enables care teams and administrators to make data-driven decisions. The practitioner registry 1914 (e.g., registry 152) may be used to manage information about care providers, including their specializations, active or inactive statuses, and assigned roles. This registry dynamically links practitioners to relevant workflows. The care plan library 1918 (e.g., care plan library 154) provides pre-configured templates for psychiatric and lifestyle care, cardiovascular care, endocrinology care, geriatric care, pediatric care, neurologic care, oncology care, disease management care, primary health care, or other health care. These templates can be customized in real-time by utilizing the patient health data (i.e., the biometric data, the historical medical data, and the patient-reported symptoms) and clinician inputs. For example, clinicians may use the clinician interface 112 to adjust the frequency of therapeutic sessions, update recommended lifestyle interventions, or add/remove milestones based on a patient's progress. Real-time customization is facilitated by integration with the patient registry 1916 (e.g., patient data repository 106), which ensures that up-to-date patient health data, such as changes in medical conditions or new diagnostic results, is automatically reflected in the care plan. The patient registry 1916 acts as the central repository for patient data, including demographics, payer information, assigned care teams, and service line history. This registry ensures a unified view of patient records, accessible across modules. The repositories integrate with workflows to automate document handling and reporting. Security and compliance module 1920 ensures adherence to regulatory requirements, such as HIPAA compliance. The security and compliance module 1920 employs role-based access, data encryption, and backup mechanisms to safeguard sensitive information. The knowledge management repositories 1922 manage clinical notes, assessment reports, claims data, and referral outcomes.

The data flow and integration within this architecture enable seamless execution of workflows to enhance patient care and operational efficiency. During patient onboarding, data collected is stored in the patient registry and linked to care teams, facilitating personalized care delivery. In the care plan development phase, templates from the care plan library are customized and shared with patients and clinicians for effective implementation. Service delivery is streamlined as care teams manage tasks and monitor progress using communication tools, while patients access real-time updates through dedicated portals. For claims and billing, time-tracking data is utilized to generate claims, which are efficiently processed using automated workflows. Additionally, aggregated data is visualized through reporting and analytics, providing valuable insights into patient outcomes, team performance, and overall system efficiency.

The system 100 is designed with interoperability as a core feature, ensuring seamless integration with FHIR (Fast Healthcare Interoperability Resources) servers, which are widely used for Electronic Health Record (EHR) data exchange. The system's ability to migrate to a FHIR-compliant structure allows it to communicate with external healthcare networks and national health exchanges, making patient data easily portable while maintaining data integrity and compliance. This migration process involves mapping existing data structures (such as IMH PatientRegistry, ServiceLineHistory, and PatientCarePlanMaster) to FHIR Resources, enabling standardized, real-time data exchange with external healthcare providers.

The migration process begins by identifying and mapping existing data lists to FHIR Resources. For example, the IMH PatientRegistry is mapped to the FHIR Patient Resource, ensuring that patient demographic information such as name, birthdate, gender, and contact details follows the FHIR standard format. Similarly, the PatientCarePlanMaster is linked to the FHIR CarePlan Resource, which allows patient treatment plans, goals, and tasks to be structured in a way that aligns with healthcare interoperability requirements. ServiceLineHistory is mapped to the FHIR ServiceRequest Resource, allowing the system to track patient services (such as psychiatry, wellness coaching, or lifestyle interventions) and ensure that service requests are standardized. Additionally, the TimeTracker is aligned with the FHIR Task Resource, enabling accurate tracking of patient consultations, wellness sessions, and psychiatric evaluations.

Once data mapping is completed, the data export process ensures that system data is converted into FHIR-compliant JSON format, allowing it to be transferred seamlessly to an FHIR server using security software and secure software programs to extract patient data and structure it according to FHIR specifications. Each data point, such as patient demographics, care plan goals, and service requests, is mapped to its respective FHIR attributes to ensure consistency and integrity. The system also employs FHIR Structure Validation Tools to verify that exported data adheres to FHIR standards before being uploaded to the server.

Integration with the FHIR server is the final step, enabling real-time data synchronization between the system and external healthcare systems. This is achieved through API integration, where the system interacts with the FHIR API to send, retrieve, and update patient records dynamically. For instance, when a patient's care plan is modified within the system, the update is automatically reflected in the FHIR CarePlan Resource on the server. Similarly, any external updates, such as new referrals or service requests, are incorporated into the patient registry, ensuring a two-way data flow that keeps all systems up to date.

FIG. 20 illustrates the interaction between different registries within the patient care management platform, demonstrating how various components work together to streamline patient onboarding, care coordination, and billing. The process begins with patient enrollment, where individuals either self-enroll through the patient portal (or app) or are referred by their primary care physicians (PCPS). Their demographic and medical details are recorded in the patient registry 2006, which serves as the central hub for managing patient information, care history, and care team assignments. This registry continuously updates and stores data, ensuring that all patient interactions are logged and accessible for future reference.

The practitioner registry 2008 operates in coordination with the patient registry 2006, dynamically assigning care providers based on their specialization and availability. This bidirectional interaction ensures that patients are matched with suitable practitioners while also keeping provider schedules optimized. In parallel, the claims registry 2012 handles the financial aspects of patient care. It collects data from both the patient registry 2006 and the time tracker 2010 to manage billing, claim submission, and payment reconciliation. The time tracker 2010 plays a role in ensuring accurate billing by logging the duration of care activities, which then informs the claims registry 2012 for invoicing and reimbursement purposes.

Additional components support care delivery by providing structured assessments and treatment plans. The system integrates assessment data 2002, such as PHQ-9 and GAD-7 scores or other health-based assessment, which guide care teams in designing personalized treatment plans. These assessments feed into the patient registry 2006, contributing to an evidence-based approach to patient management. The care plan library 2004 further enhances this process by offering predefined treatment protocols that ensure consistency across different cases. The time tracker 2010 also interacts with the patient registry 2006 to maintain accurate records of patient interactions and service durations.

The registries and intermediate components collaborate to enhance patient outcomes, optimize provider assignments, and streamline financial operations. By centralizing patient and practitioner data while automating claims processing and care tracking, the patient care management platform ensures efficiency, accuracy, and improved healthcare delivery.

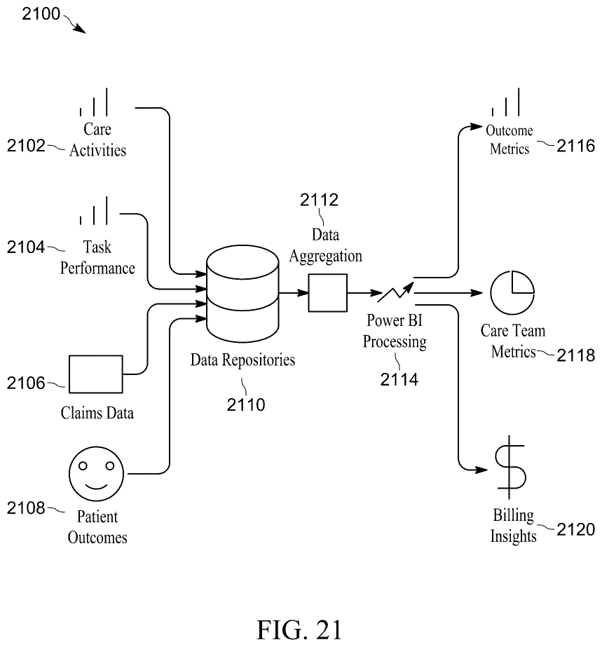

Referring now to FIG. 21, an example data flow diagram 2100 of aggregation and processing of data from care activities, task performance, claims data, and patient outcomes into clinical insights using business intelligence dashboards, is illustrated, in accordance with an embodiment of the present disclosure.

At care activities 2102, data is collected from the patient registry and practitioner registry, including information related to care plans, clinical interventions, and overall healthcare delivery activities. At task performance 2104, escalations, task updates, and completion metrics are logged through automation tools such as Microsoft's Power Automate®. These data reflect the efficiency and responsiveness of task management workflows. The claims data 2106 includes data related to insurance claims, approvals, and denials, providing insight into financial workflows and reimbursement trends. At patient outcomes 2108, health outcomes such as recovery rates, adherence to treatment plans, and patient satisfaction metrics are tracked.

These data streams are stored in the data repositories 2110, which serve as centralized storage hubs for real-time updates from various system activities. The data repositories ensure secure and structured storage, maintaining the integrity and accessibility of logged information. The stored data undergoes data aggregation 2112, where it is combined and processed from multiple sources, such as patient records, task logs, and financial data, to generate a unified dataset. The aggregated data is then processed using business intelligence processing 2114, where advanced data visualization and analytics tools are applied to generate insights. These insights are categorized into one or more dashboards, for example, outcome metrics 2116, care team metrics 2118, and/or billing insights 2120. The outcome metrics 2116 displays care effectiveness metrics, such as patient recovery rates, adherence to care plans, and health improvement statistics, providing clinicians with clinical insights into treatment success. The care team metrics 2118 highlight task completion rates, escalation resolution times, and team performance, enabling supervisors to monitor and optimize workforce efficiency. The billing insights 2120 provides a view of claims approval rates, revenue trends, and financial health, allowing administrative staff 1810 to manage financial workflows effectively.

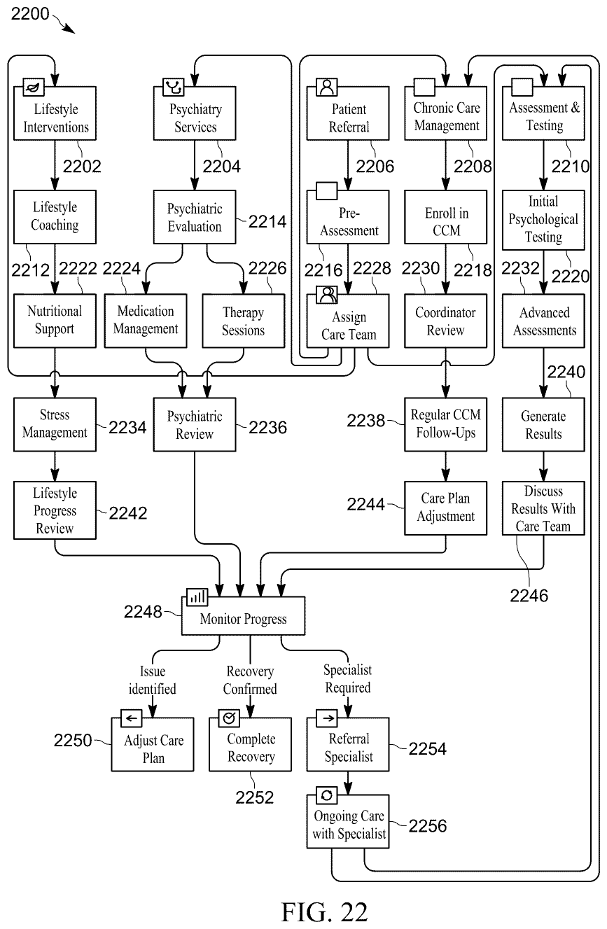

Referring now to FIG. 22, an example flow diagram illustrating the particular phases of a patient journey within the patient care management platform is provided in accordance with an embodiment of the present disclosure. The flow diagram 2200 represents a structured and systematic approach to patient care, covering the entire lifecycle of patient management, beginning with the referral phase and extending through service line workflows, continuous progress monitoring, and iterative care plan adjustments to ensure optimal healthcare outcomes.

The patient journey begins with the patient referral phase 2206, where individuals may be referred by primary care physicians (PCPs), specialists, or through self-enrollment. This stage is represented in the diagram as the “patient referral” 2206 and “pre-Assessment” 2216 steps, ensuring that essential patient data is collected and stored in the patient registry for seamless care coordination. Once the referral is received, the pre-assessment phase 2216 involves preliminary clinical evaluations using standardized assessment tools such as PHQ-9 and GAD-7 or other health-based assessment, which help determine the appropriate service lines and facilitate the care team assignment. The diagram illustrates the transition from “pre-assessment” 2216 to “assign care team” 2228, reflecting the platform's ability to dynamically allocate healthcare professionals based on patient needs.

Following the assignment, the care team, which may consist of psychiatrists, therapists, and health coaches, takes over the management of the patient's treatment plan. The patient journey then diverges into multiple service lines, each addressing different aspects of care. Psychiatry services 2204 focuses on mental health treatment, including psychiatric evaluations 2214, medication management 2224, therapy sessions 2226, and periodic psychiatric reviews 2236. This structured workflow ensures that patients receive continuous, specialized care throughout their treatment. Additionally, lifestyle intervention module 2202 offers guidance in areas such as lifestyle coaching 2212, nutritional support 2222, stress management 2234, and ongoing progress tracking, catering to patients who utilize behavioral and lifestyle modifications. For individuals needing long-term support, the Chronic Care Management (CCM) 2208 program provides continuous monitoring and adaptive care strategies, including enrollment 2230, periodic coordinator reviews 2218, regular CCM follow-ups 2232, and care plan adjustments 2244 to ensure sustained health outcomes. The system also integrates assessment and testing services 2210 to facilitate comprehensive psychological and cognitive evaluations, including initial psychological testing 2220 and advanced assessments, which feed directly into personalized treatment strategies based on diagnostic findings.

One example aspect of the patient journey is cross-service coordination, which ensures seamless integration across multiple service lines. The monitor progress functionality 2248 provides real-time patient status updates through interactive dashboards, enabling clinicians to assess treatment effectiveness and identify areas requiring intervention. The care plan adjustments 2244 allows healthcare providers to modify treatment strategies based on patient progress, ensuring that care remains responsive and individualized. If a patient achieves full recovery, the journey concludes with “complete recovery” 2252, signifying the end of active treatment. However, if further intervention is required, the system enables care plan refinements 2250 or facilitates a “referral to specialist” 2254, ensuring continuity of treatment through external healthcare providers while maintaining oversight from the original care team. For cases requiring ongoing specialist care, “ongoing care with specialist” 2256 ensures continued management of the patient's condition.

The system also accounts for edge cases and task escalations, ensuring that disruptions in care are minimized. If a patient requires multiple referrals, such as concurrent treatment in psychiatry services 2204 and chronic care management 2208, the system effectively manages parallel workflows, preventing service overlap or administrative inefficiencies. In cases where a patient drops out mid-journey, automated reminders and follow-ups are triggered to re-engage the patient and reduce attrition. Additionally, if a service line experiences overload, the platform dynamically reassigns practitioners, balancing workloads and maintaining efficient service distribution without compromising patient care.

The process visually encapsulates a structured and adaptive patient journey facilitated by the patient care management platform. Through its data-driven approach, the system seamlessly integrates referral intake, pre-assessment, multi-service treatment workflows, continuous progress tracking, and iterative care modifications to ensure optimal patient outcomes. By dynamically managing practitioner assignments, streamlining service coordination, and implementing automated intervention mechanisms, the platform enhances care delivery efficiency while maintaining a patient-centric, scalable, and outcome-focused care management model.

The Lifestyle Intervention module 2202 within the system is designed to help patients incorporate healthy habits into their mental and/or physical health treatment. By integrating changes related to diet, exercise, and stress management, this module ensures that lifestyle modifications are an active part of a patient's care plan. These interventions are continuously tracked and adjusted based on patient progress, supporting the concept of lifestyle psychiatry, which focuses on both physical and emotional well-being for long-term health improvements.

The system embeds lifestyle interventions directly into patient care plans, allowing psychiatrists, health coaches, and care teams to work together in real-time to monitor progress. Patients receive personalized goals, such as nutritional support 2222, exercise targets, or stress management 2234 like mindfulness or meditation. These goals can be customized for individual patients or selected from predefined templates based on evidence-based practices in Lifestyle Psychiatry. The system automatically generates daily tasks for patients, such as completing mindfulness exercises or tracking diet changes, using software/dashboards to ensure seamless integration into the patient's daily routine. The status of these tasks is monitored, allowing care teams to adjust goals based on real-time patient feedback. If a patient struggles with a certain activity, such as regular meditation, the care team can modify the plan to better fit the patient's needs.

To monitor lifestyle outcomes, the system collects both biometric data (such as heart rate, sleep patterns, and physical activity levels) and behavioral data (such as mood tracking and self-reported stress levels). This data is gathered from wearable devices or entered manually by patients or care team members, ensuring that mental health progress and/or health progress, in general, can be correlated with physical health improvements. Dashboards aggregate this information at both the individual and population levels, providing insights into how lifestyle changes are affecting mental health outcomes or health outcomes, in general. These dashboards display metrics, such as the percentage of patients meeting their goals, trends in sleep improvement, and engagement rates for activities like mindfulness practice or exercise.

Successful lifestyle interventions require collaboration among multiple healthcare roles, including health coaches, care managers, and psychiatrists. health coaches lead patient engagement in activities like diet changes and physical exercise, while care managers and psychiatrists ensure that these lifestyle modifications align with psychiatric treatments. The system employs role-based permissions, allowing health coaches to access lifestyle-related data while restricting access to sensitive psychiatric information, ensuring privacy and compliance. Additionally, automated workflows assign tasks across roles, such as scheduling wellness check-ins or tracking patient adherence to interventions.

Patient engagement is a part of the system 100, as patients are encouraged to self-report their daily activities, such as exercise routines or mindfulness sessions, using the patient portal or patient app. This real-time data is synced with care plans, allowing care teams to monitor adherence and provide feedback. To further encourage participation, the portal provides educational resources, including articles, videos, and interactive tools focused on diet, exercise, and stress management. Additionally, motivational prompts and reminders help patients stay engaged by suggesting ways to improve sleep quality or maintain a mindfulness routine.

The effectiveness of lifestyle interventions is continuously evaluated through reports and analytics. Care teams and health administrators can track adherence rates to lifestyle changes and assess their impact on mental and physical health. If data shows that patients are struggling with a certain intervention, the system allows for care plan adjustments 2244 to make goals more achievable, such as modifying an exercise routine to include less intensive activities. These adjustments are tracked and analyzed to ensure that changes lead to better outcomes.

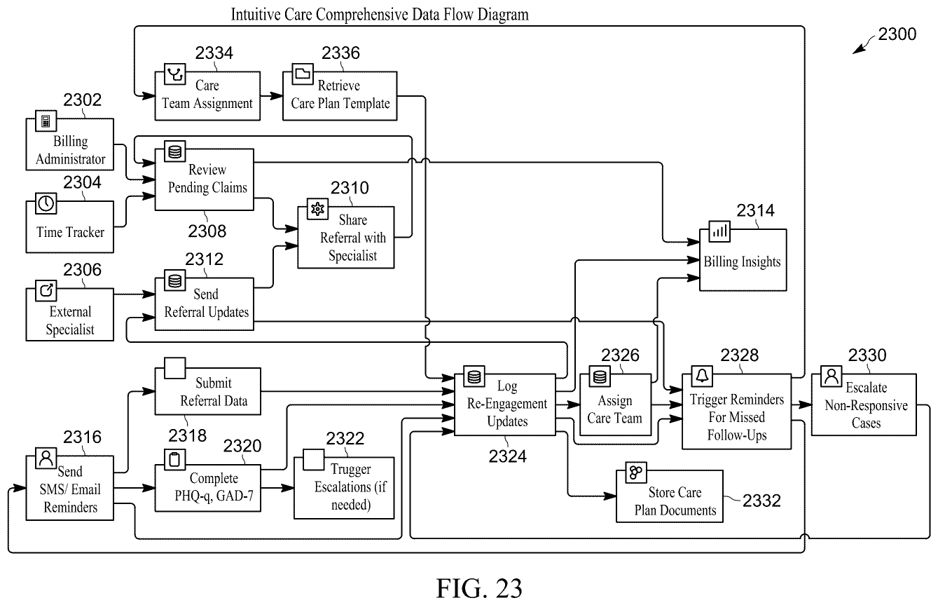

Referring now to FIG. 23, an example data flow diagram 2300 illustrates the structured movement of data across various workflows, enabling seamless coordination, real-time insights, and operational efficiency in accordance with an embodiment of the present disclosure. The diagram demonstrates how different system components dynamically interact to facilitate efficient patient management, care coordination, and financial operations, ensuring that all processes function in a streamlined and integrated manner.

The data flow initiates at the Patient Referral and Initial Assessments phase, where patient information is submitted to begin the intake process. As illustrated in the “submit referral data” 2320 step, this ensures that the referral request is logged and processed. The system then communicates real-time updates to specialists using the example sequence “send referral updates” 2312→“Share Referral with Specialist” 2310, allowing immediate access to referral details. Following the referral, patients undergo initial clinical assessments, such as PHQ-9 and GAD-7 2318 or other health-based assessment, which evaluate their condition and determine a suitable care path. These assessments provide input for treatment planning, ensuring that each patient receives a care approach tailored to their needs.

Once the intake and assessment phase is completed, the data flow advances to care team assignment and plan management, where specialists are dynamically assigned based on assessment results. The “assign care team” 2326 step ensures that appropriate healthcare professionals are allocated for each case, optimizing provider-patient matching. Subsequently, a predefined “care plan template” 2336 is retrieved and structured into a standardized treatment pathway. To ensure accessibility and continuity, all care plans are securely stored through the “store care plan documents” 2332 function, allowing providers to reference and update treatment plans as needed. This structured approach to care planning enhances treatment consistency and ensures proper documentation for compliance and care coordination.

Throughout the treatment process, patient engagement and follow-ups play a role in ensuring adherence to prescribed care plans. The system continuously monitors engagement levels by capturing “re-engagement updates” 2324, which track patient participation and response to treatments. To prevent disengagement, the platform automatically triggers reminders from the “trigger reminders for missed follow-ups” 2328 step, encouraging patients to remain active in their treatment plans. If a patient remains unresponsive, the case is escalated through “escalate non-responsive cases” 2330, enabling care teams to intervene and take corrective actions to re-engage the patient before critical gaps in care occur.

In parallel with clinical workflows, billing and claims processing ensures efficient financial operations by routing billing data to the billing administrator 2302, who is responsible for overseeing claim approvals and financial reconciliations. Within this framework, the “review pending claims” 2308 step ensures that all claims undergo verification, validation, and approval before submission to payers, reducing errors and delays in reimbursement. Additionally, “billing insights” 2314 provides real-time financial visibility, offering administrators an up-to-date overview of claim statuses, revenue management, and outstanding financial transactions, thereby improving financial decision-making and transparency.

In scenarios requiring specialized interventions or escalations, the escalations and external coordination workflow ensure that necessary actions are taken efficiently. If an urgent issue arises, the system initiates “trigger escalations (if needed)” 2322, prompting intervention from senior care managers or external specialists. The data flow also facilitates coordination with external healthcare providers by allowing referral updates to be shared with external specialists through “external specialist” 2306→“send referral updates” 2312, ensuring a smooth transition of care and preserving continuity in treatment delivery.

In some embodiments, the system includes a testing and monitoring (TM) service line designed to conduct regular mental and/or physical health assessments and screenings, providing data to track patient progress and refine treatment plans. This service helps care teams make informed decisions by utilizing health screening tools such as PHQ-9 for depression, GAD-7 for anxiety, and MDQ for mood disorders, or another health-based assessment. These assessments are conducted using a form or other linked access to information, with results automatically linked to the patient's care plan for ongoing evaluation.

The TM service also monitors lifestyle interventions, but this is done manually rather than through automated tracking. Patients or care teams input lifestyle metrics, such as adherence to exercise or mindfulness routines, into the system. The system 100 may trigger workflows based on task completion and patient-reported outcomes. For example, if a patient fails to follow a prescribed mindfulness routine, the system alerts the care manager for follow-up.

To ensure consistency in testing, the system provides automated reminders to both patients and care teams about upcoming assessments. These reminders managed through system 100, help maintain regular screening schedules and prevent delays in evaluations.

The care team-including care managers, psychiatrists, and health coaches-plays a role in interpreting test results and adjusting care plans manually. Unlike future versions, the current system does not use AI-driven decision-making but relies on expert human judgment. Instant messaging platforms may facilitate communication among care team members, ensuring that all relevant professionals are informed of changes in patient health.

For reporting and analytics, the system utilizes one or more dashboards to provide both patient-specific and population-level insights. Individual reports track assessment scores over time, helping care teams monitor progress, while aggregated reports highlight broader trends across the patient population.

In some embodiments, the TM service may include remote patient monitoring (RPM) with biometric tracking through wearable devices, allowing automatic data collection for factors like heart rate and activity levels. Additionally, predictive analytics and AI-based forecasting will eventually be integrated to anticipate patient health trends based on historical data. However, the current version focuses solely on manual data collection and static assessments to ensure care teams receive reliable periodic data for decision-making.

In some embodiments, the system comprises a telehealth and remote care integration module that enhances the delivery of virtual healthcare services, allowing patients and care teams to connect remotely while ensuring high-quality care. This module provides a seamless and secure platform for virtual consultations, remote communication, and future advancements in remote patient monitoring.

The system integrates with widely used communication tools like instant messaging platforms, telephone platforms, and a secured text messaging platform. These integrations enable real-time video consultations, voice calls, and secure text-based communication between patients and providers. This ensures that patients can access healthcare services conveniently from their homes while care teams can conduct virtual assessments, follow-ups, and consultations without requiring in-person visits.

In some embodiments, the module is configured to include remote patient monitoring (RPM) capabilities and the integration of emerging remote care models. RPM will allow healthcare providers to track patients' vital signs, symptoms, and adherence to treatment plans in real-time through connected devices and smart technology. This proactive approach will help detect health issues early, reduce hospital readmissions, and improve long-term patient outcomes.

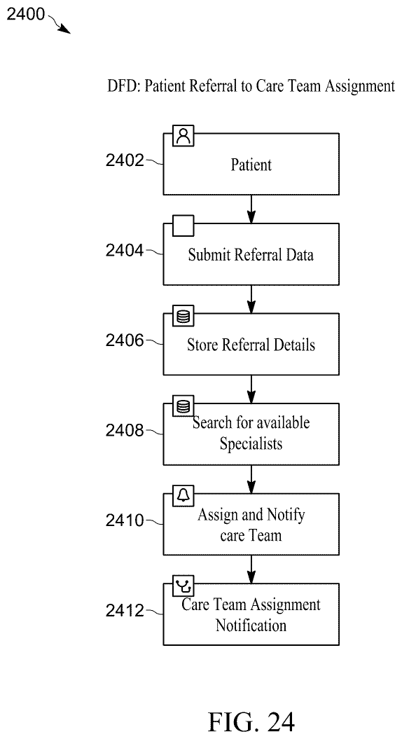

Referring now to FIG. 24, an example data flow diagram illustrates the flow of patient referral to care team assignment, in accordance with an embodiment of the present disclosure. The process 2400 represents a structured and automated process that ensures patient referrals are efficiently processed, specialists are dynamically assigned, and care teams are promptly notified.

The data flow initiates with the patient referral submission 2402, wherein the patient submits referral data 2404, triggering the workflow. This action marks the beginning of the referral process, ensuring that the patient's need for care is recorded and processed in real-time.

Following submission, the storing referral details phase 2406 is executed, where the patient registry serves as the central repository for securely storing referral details. The patient registry maintains the integrity of patient records, ensuring that demographic data, medical history, and referral specifics are accurately documented.

Once the referral data is securely stored, the system proceeds to search for available specialists 2408 by dynamically querying the practitioner registry. The practitioner registry 152 plays a role in identifying appropriate providers based on availability, specialization, and patient needs. This automated process optimizes resource allocation by ensuring that relevant and available care providers are considered for assignment.

Upon identification of the appropriate specialist or care team, the workflow advances to assigning and notifying the care team 2410. At this stage, the system assigns the selected specialist(s) and generates an automated notification, ensuring that all necessary personnel are informed of their newly assigned case. This mechanism eliminates inefficiencies associated with manual referrals, reducing response time and improving care coordination.

The final step in the process is care team assignment notification 2412, where the assigned care team formally receives a notification confirming their responsibility for the referred patient. This step ensures accountability within the care management system and enables practitioners to take immediate action regarding patient assessment and treatment planning.

The data flow diagram effectively visualizes the seamless handling of referrals and automated care team assignment, ensuring minimal delays in patient care initiation. The patient registry 106 and practitioner registry 152 facilitate secure data storage, real-time provider matching, and efficient case allocation. Furthermore, any inefficient manual steps within this workflow can be flagged for automation or optimization, reinforcing the objective of streamlining referral management and enhancing operational efficiency.

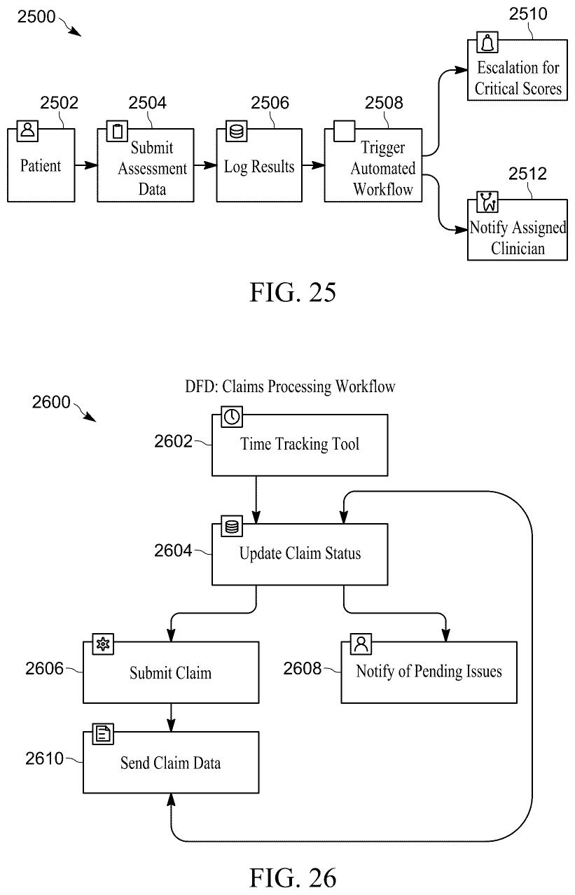

Referring now to FIG. 25, an example flow diagram for the patient health data processing within the clinical assessment system 100, is illustrated, in accordance with an embodiment of the present disclosure. The flow diagram 2500 represents a seamless integration of the patient health data 2502 into clinical insights and workflows for clinical decision-making. The process includes receiving assessment data 2504 (i.e., patient health data), related to a patient, through the patient interface 110. These data may include responses to clinical surveys, diagnostic assessments, or lifestyle-related inputs. Once submitted, the data is logged 2506 into a secure repository, ensuring the data is stored in the Patient Registry for subsequent analysis and reference. This secure logging ensures compliance with data protection standards and allows for real-time or retrospective analysis.

Following data logging, an automated workflow is triggered 2508 based on pre-configured thresholds or criteria within the system. These workflows are designed to detect critical scores such as high-risk PHQ-9 scores (e.g., ≥20, indicating severe depression), elevated GAD-7 scores (e.g., ≥15, reflecting severe anxiety), or abnormal biometric readings (e.g., heart rate variability below a certain threshold) in the assessment data. If critical scores are identified, the workflow enables escalation 2510 may ensure that urgent cases are flagged for immediate action. In parallel, the workflow notifies the assigned clinician 2512 to enable timely review and intervention tailored to the needs of the patient.

The escalation workflow ensures that any missed or delayed psychiatric or wellness-related tasks are promptly identified and addressed to maintain continuity of care. Administrators play a role in monitoring these escalations, ensuring that appropriate follow-ups are conducted to keep patients engaged with their care plans. The system flags missed wellness tasks, such as mindfulness sessions or wellness check-ins, in patient interface 110, for example, which then escalates the issue to the responsible team member, such as a health coach. This automated escalation triggers follow-up workflows designed to re-engage the patient and prevent lapses in their wellness routines. For high-risk patients who are not adhering to their mindfulness programs or other wellness interventions, the system issues additional alerts to the operations lead, prompting more intensive engagement strategies to reinforce participation.

Beyond wellness-related escalations, the system also prioritizes clinical-critical escalations for high-risk psychiatric cases. Tasks such as overdue medication reviews or unaddressed high PHQ-9 scores, which indicate worsening mental health, or other health-based assessment indicating worsening health are escalated with the highest urgency. These escalations ensure that care managers and supervisors are immediately notified, allowing them to intervene before the patient's condition deteriorates further. The escalation workflow is integrated with dashboards, providing real-time visibility into outstanding critical tasks, and enabling administrators and clinicians to track and resolve urgent cases efficiently. This structured approach to escalation enhances patient safety, promotes adherence to treatment plans, and ensures that both psychiatric and wellness-related concerns are proactively managed.

Referring now to FIG. 26, an example data flow diagram illustrating the claim processing workflow is provided in accordance with an embodiment of the present disclosure. The process 2600 represents the structured movement of claim-related data, ensuring billing accuracy, real-time status updates, and seamless integration with external payor systems to facilitate efficient claim management.

The workflow initiates with the time tracking tool 2602, which records billable activities associated with patient care. By capturing these activities in real-time, the system ensures that claim submissions are based on accurately logged services, reducing discrepancies in medical billing and preventing potential claim denials due to missing or incorrect information. The recorded billing data is then transmitted to the claims registry, where the system updates the claim status dynamically 2604. These real-time status updates are utilized by billing administrators, providing visibility into claim progression, pending actions, and potential issues requiring intervention.

Once the claims registry is updated, the system proceeds to the submitting 2606 and sending claim data phase 2610. At this stage, validated claims are transmitted to external payor systems using FHIR API integration. This ensures real-time data exchange between the healthcare platform and insurance providers, expediting claim approvals and reimbursement processing. By automating the claim submission process, the system enhances operational efficiency and minimizes delays caused by manual data entry or paper-based submissions.

If any issues arise during claim processing, such as missing documentation, incorrect coding, or validation errors, the system automatically triggers pending issue notifications 2608. These notifications alert billing administrators and relevant stakeholders to potential claim discrepancies, allowing for prompt resolution before the final claim submission. Automating this notification process reduces administrative workload, prevents claim rejections, and ensures compliance with payor-specific billing requirements.

The claims processing workflow provides several functionalities to optimize billing operations. claims registry management ensures that billable activities are accurately logged, tracked, and resolved, minimizing revenue loss due to claim errors. FHIR API Integration facilitates real-time communication with payor systems, reducing processing time and improving claim approval rates. Additionally, billing insights using dashboards offers comprehensive financial oversight by providing billing administrators with a dashboard view of claim statuses, pending actions, and revenue metrics, enhancing decision-making and financial transparency.

This workflow efficiently manages claims from submission to resolution, ensuring billing transparency, automated tracking, and seamless integration with external insurance systems. By leveraging FHIR API for automated payor communication and dashboards for financial visibility, the system enhances billing accuracy, claim efficiency, and reimbursement speed.

Furthermore, the system may further provide automation of pending issue notifications, allowing for quicker resolutions and reducing manual intervention. By improving the automated error detection and resolution mechanism, the system could further streamline claim processing, enhance compliance, and optimize overall billing efficiency.

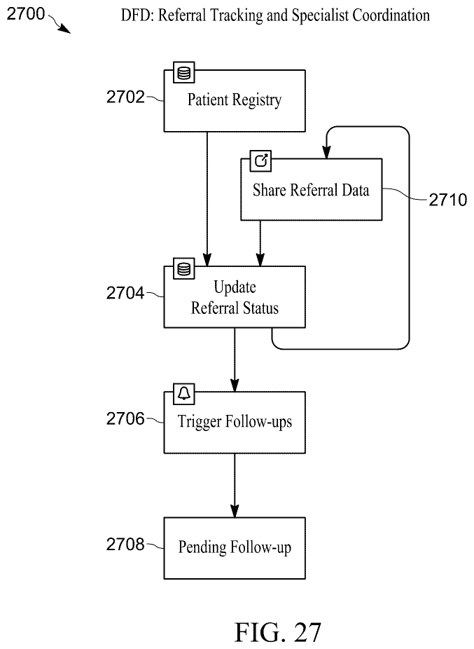

Referring now to FIG. 27, an example flow diagram illustrating a patient referral tracking and coordination workflow is provided in accordance with an embodiment of the present disclosure. The workflow 2700 is designed to ensure efficient referral management, real-time tracking, and automated follow-ups, thereby streamlining the process of assigning, monitoring, and completing patient referrals without administrative delays.

The referral tracking process begins with the patient registry 2702, which serves as a centralized repository for storing referral-related data, including patient demographics, referral history, and ongoing case status. By maintaining a structured and accessible record of referrals, the system facilitates seamless care coordination and prevents information silos that can arise when multiple providers are involved in a patient's treatment.

Upon receiving a referral request, the system automatically directs the referral data to relevant specialists 2710. This assignment is performed dynamically based on a range of factors, including provider availability, specialization, and the urgency of the referral. By leveraging an intelligent referral matching mechanism, the system ensures that patients are connected to appropriate specialists in a timely manner, reducing delays in specialist access and improving the overall efficiency of the referral process.

Once a referral is processed or reviewed, the system updates the referral status in real-time 2704, enabling continuous tracking by care teams, administrative staff, and referring providers. This transparency ensures that all stakeholders have visibility into the progress of each referral, minimizing the risk of referrals being misplaced, forgotten, or subject to administrative bottlenecks.

To further enhance efficiency, the system integrates an automated follow-up mechanism that proactively monitors referrals that remain unaddressed or pending beyond a predefined threshold. If a referral is not acted upon within the expected timeframe, the system triggers a notification 2706 prompting the appropriate stakeholders to take corrective action. This feature helps eliminate unnecessary delays, ensuring that referrals are actively managed and do not stall at any stage of the process.

In cases where additional action is required before a referral can proceed, the system moves the referral into a pending follow-up state 2708, where it is continuously monitored until it is either resolved or escalated for further review. This step ensures that no referral is left unattended, allowing care teams to intervene as needed to address any outstanding issues.

By integrating real-time referral tracking, automated follow-ups, and a centralized referral management system, the disclosed workflow optimizes specialist coordination, reduces administrative workload, and enhances overall patient care efficiency. Through structured referral tracking and intelligent automation, the system ensures that referrals are completed promptly and accurately, ultimately improving patient outcomes and streamlining healthcare operations.

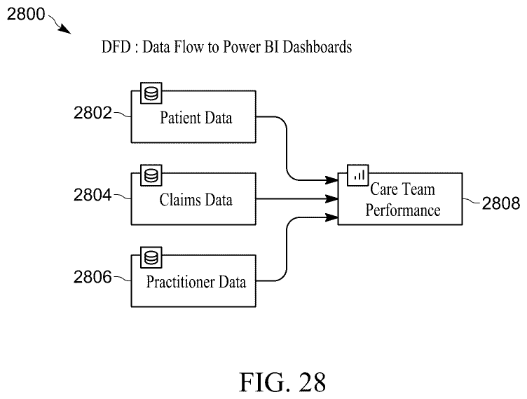

Referring now to FIG. 28, an example data flow diagram 2800 depicting the integration of various data streams into business intelligence dashboards for clinical insights, is illustrated, in accordance with an embodiment of the present disclosure. FIG. 28 highlights the aggregation of patient data 2802, claims data 2804, and practitioner data 2806 into a system to evaluate and enhance care team performance 2808.

The process includes the patient data 2802, which includes demographic details, health records, and outcomes from clinical interactions. These data are continuously updated within the patient registry and securely integrated into the analytical framework. Claims data 2804 provides financial and administrative information, such as billing statuses, payment records, and claim approvals. These data ensure visibility into the revenue cycle and identify opportunities for efficiency improvements. Practitioner data 2806 encompasses care team details, including task completion rates, specializations, and performance metrics, enabling a comprehensive understanding of resource utilization. These datasets are aggregated and fed into the care team performance module 2808, which powers real-time dashboards. The business intelligence dashboards serve as a unified platform for decision-making by visualizing key performance indicators such as patient outcomes, task efficiency, and financial metrics.

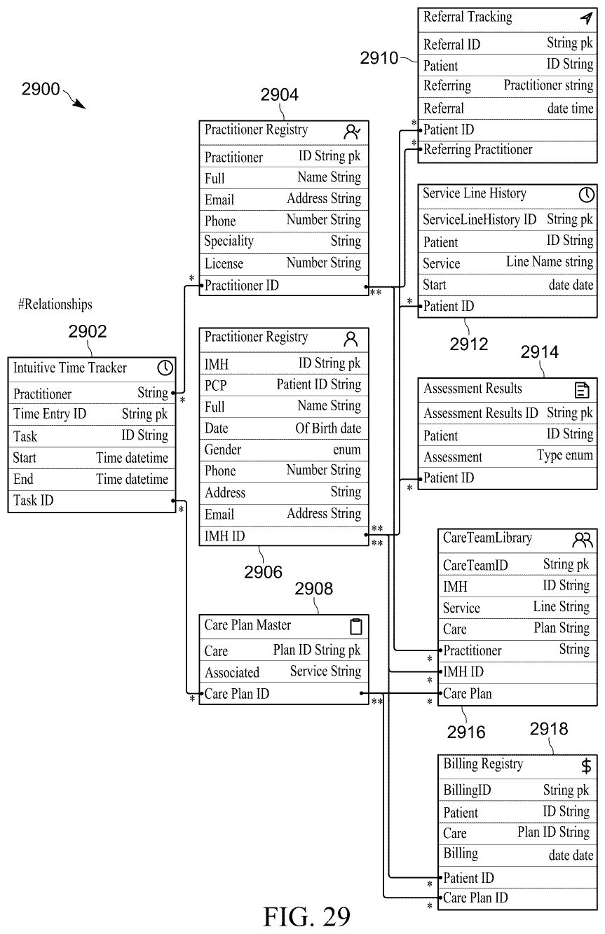

Referring now to FIG. 29, an example flow diagram illustrating the data structure of the patient care management platform is provided in accordance with an embodiment of the present disclosure. The process 2900 highlights the entities and their relationships, showcasing how patient data, practitioner details, referrals, assessments, billing, and care plans are interconnected to facilitate seamless healthcare coordination and data management. The Entity-Relationship Diagram (ERD) ensures data integrity, scalability, and real-world interaction mapping, enabling efficient healthcare management by defining structured relationships and enforcing constraints that eliminate inconsistencies and enhance future system expansions.

At the core of this system is the patient registry 2906, which serves as the central repository for storing patient demographics, medical history, ongoing treatments, and care team assignments. The practitioner registry 2904 maintains information on healthcare providers, their specializations, licenses, and availability, ensuring that referrals and assignments are dynamically managed. The Care Plan Master stores structured care plans, linking them with both the Care Team Library and the Billing Registry to ensure that treatment plans are correctly implemented and financial processes align with clinical workflows.

The patient registry 2906 interacts with multiple entities to streamline healthcare operations. It maintains a direct connection with the service line history 2912, tracking patient interactions across different service offerings. It also links to the assessment results 2914, ensuring that diagnostic evaluations are integrated into the care process. Furthermore, its connection with the billing registry 2918 ensures that all financial transactions related to patient care services are systematically recorded, supporting accurate claims processing. Similarly, the practitioner registry 2904 plays a role in facilitating provider coordination, linking with the referral tracking system 2910 to route patients to appropriate specialists and ensuring that practitioner assignments align with patient needs. Additionally, the care team library 2916 is integrated with the practitioner registry 2904, allowing the system to dynamically assign providers to multidisciplinary care teams and ensuring structured, team-based patient management.

The billing registry 2918 is another vital component of the system, synchronizing financial records with the care plan master 2908 to ensure that all treatment plans are accurately billed and compliant with healthcare reimbursement standards. By establishing these structured connections, the platform enhances centralized data management, optimized referral handling, and seamless financial operations. This interconnected framework ensures that patient, provider, and billing information remains synchronized and easily accessible, allowing for real-time tracking, automated referral handling, and transparent billing processes.

The ERD representation of the patient care management platform supports scalability and system expansion, allowing for future enhancements and the integration of additional healthcare services. By defining robust data relationships, the system ensures that all healthcare activities, from patient assessments and referrals to care team assignments and financial transactions, remain well-coordinated and efficiently managed. This structured approach enhances operational efficiency, promotes evidence-based care, and ensures financial accountability, ultimately contributing to improved healthcare delivery and optimized resource utilization.

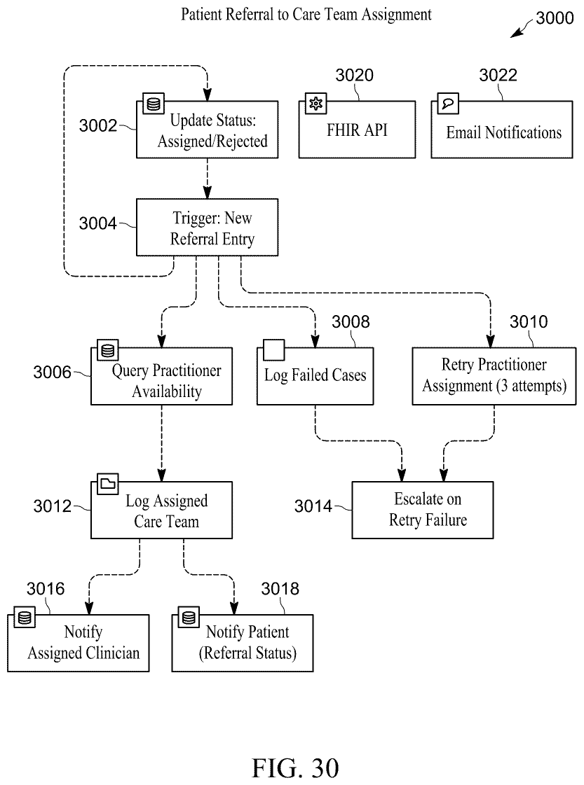

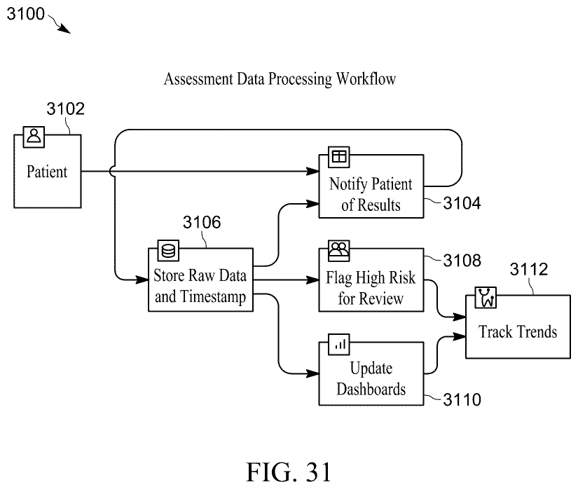

Referring now to FIG. 30, an example flow diagram illustrating the patient referral to the care team assignment process is provided in accordance with an embodiment of the present disclosure. The process 3000 maps out the combination of automated and manual steps necessary to ensure the seamless and efficient assignment of patients to the appropriate care team. The system is designed to enhance workflow automation by streamlining task delegation, exception handling, and notification processes, ensuring that referrals are processed without unnecessary delays or inefficiencies.

The workflow begins with the triggering of a new referral entry 3004 into the Patient Registry, which can occur through manual input by healthcare staff or using FHIR API integration from external systems. Once a referral is logged, the system automatically initiates a query 3006 within the practitioner registry to identify an available specialist based on service line specialization, workload capacity, and practitioner availability. If a suitable provider is found, the system proceeds with automated assignment, ensuring a smooth transition from referral intake to care team allocation. However, if no provider is available at the initial attempt, the system is programmed to retry the query up to three times before escalating the issue for manual intervention. The system updates the status 3002, whether the practitioner is assigned or rejected.

In cases where practitioner assignment fails after three attempts 3010, the system logs the failed cases 3008 and escalates them for manual review and intervention by a care coordinator 3014. This escalation mechanism ensures that patients are not left without appropriate care due to temporary practitioner unavailability. Once a practitioner is successfully assigned, the system updates the care plan library with the newly designated care team information and logs the referral status in the Patient Registry to maintain a structured and traceable record of assignments.

Following the successful assignment of a care team 3012, the system triggers notifications to various stakeholders to ensure all parties are informed of the referral outcome. The assigned clinician receives an internal system alert 3016, the patient is notified by email or patient portal 3018, and the referral source is updated on the status of the referral to ensure transparency in care coordination.

To address error handling and escalation logic, the system incorporates predefined failure detection mechanisms. If a referral entry is missing required fields, a validation error is triggered, prompting staff to correct the input before proceeding. If no available practitioners are found after three automated attempts, the system flags the issue for escalation to a regional supervisor if the referral remains unprocessed beyond 24 hours. The system also includes a fallback mechanism that ensures, in cases where automated retries fail, a care coordinator is notified for manual intervention.

The workflow relies on several tools and integrations to maintain efficiency and accuracy. In some embodiments, Microsoft's Power Automate® is used to manage workflow automation, ensuring the timely execution of tasks. The FHIR API 3020 facilitates external data integration, allowing interoperability with other healthcare systems. The system uses email notification 3022 to notify different stakeholders. SharePoint (or another sharing site) serves as a repository for workflow logs and tracking data, maintaining audit trails for administrative review. The patient and practitioner registries provide the necessary data points for making real-time assignments based on practitioner availability and patient needs.

This automated workflow enhances referral handling efficiency by reducing manual workload, ensuring structured assignments, and improving notification accuracy. The inclusion of retry mechanisms, escalation protocols, and error handling guarantees that no referral is left unprocessed, ultimately enhancing patient care coordination and ensuring timely interventions.

Referring now to FIG. 31, an example workflow diagram 3100 for assessment data processing, showcasing the end-to-end handling of patient-submitted assessments to generate clinical insights, is illustrated, in accordance with an embodiment of the present disclosure. The workflow begins with a patient 3102 submitting an assessment, which may include standardized tools such as PHQ-9 or GAD-7, through a patient-facing interface, such as a web portal, mobile application, or clinician-guided submission. The assessment submission includes metadata such as the assessment type, patient identification, and timestamp. This information forms the foundational layer for downstream processing. The submitted data is then automatically forwarded to a storage module 3106, which securely stores the raw responses alongside the associated timestamp for traceability and compliance. This module acts as the repository for submitted assessments, ensuring data integrity and accessibility for subsequent workflows.

Upon successful submission, the system validates the assessment data to ensure that fields are populated and that responses match the expected format. For example, the system checks that numerical values are provided for all scored questions in a PHQ-9 form. If any data is incomplete or invalid, the workflow flags the submission as “incomplete” and sends a notification to the patient 3104 to resubmit the form, minimizing gaps in data processing. This validation step helps maintain the quality and accuracy of clinical insights generated later in the workflow. For example, the clinical insights may include, the severity of depressive symptoms based on PHQ-9 scores, correlations between patient-reported symptoms and historical medical data, prioritized care recommendations tailored to a patient's risk level, personalized treatment plans that address comorbid conditions, and predictive health assessments to foresee potential complications or progression in the patient's condition.

Once validated, the system processes the data by calculating assessment scores based on predefined rules. For example, in the case of PHQ-9, the system sums the individual question scores to compute a total score. This calculated score, along with the raw responses, is stored within the assessment results table in the storage module 3106. The system then analyzes the calculated score to determine the patient's risk level. For instance, a PHQ-9 score of 20 or above may be flagged as high risk, while scores in the range of 10-14 may be categorized as moderate risk. A score less than about 10 may be categorized as low risk. Based on the risk level, the system determines the appropriate course of action.

If the risk level is categorized as high or above a predefined threshold, the workflow triggers an automated flagging process 3108 to escalate the results to the assigned clinician or care team for urgent review. Notifications for flagged results are sent to the clinician through secure channels such as email, text messages, or integrated notifications in a clinician-facing dashboard. This ensures that high-priority cases receive immediate attention, minimizing the risk of delayed intervention. In parallel or sequentially, the system notifies the patient 3104 of their results along with any recommended next steps. For low-risk cases, the notification may include a reassurance message and educational resources to help the patient understand their assessment results. For moderate-risk cases, the notification may suggest scheduling a follow-up assessment or consultation. The system ensures that patient communication is clear, timely, and aligned with clinical guidelines.

The workflow further integrates advanced analytics by updating real-time dashboard 3110 with the assessment results. These dashboards provide a holistic view of trends across multiple patients, enabling clinicians and care administrators to monitor patterns such as an increase in high-risk assessments over time. The dashboards also allow for tracking individual patient progress, offering insights into the effectiveness of ongoing care plans. Additionally, the system includes a trend-tracking module 3112 that monitors historical data to identify long-term patterns and deviations. For example, a patient's scores across multiple PHQ-9 assessments or other health-based assessment may be analyzed to determine whether their condition is improving or worsening. This module provides insights for clinicians to adjust treatment plans proactively.

The workflow incorporates error-handling mechanisms to ensure reliability. If notifications to patients or clinicians fail due to technical issues, the system retries the notifications multiple times at predefined intervals. If repeated failures occur, the system escalates the issue to the system administrator for manual intervention. Similarly, if flagged assessments are not reviewed by clinicians within a predefined time frame, the system sends reminders and escalates the matter to supervisory staff to ensure timely action. To support compliance and auditing, the system logs actions and notifications in an audit trail, ensuring that steps of the workflow can be traced back for accountability. This may be used for meeting regulatory requirements such as HIPAA, which mandates secure handling of patient data.

In some embodiments, the system allows for the customization of thresholds for both psychiatric assessments and wellness metrics. These thresholds can trigger real-time alerts, ensuring that care teams receive notifications for significant changes in either psychiatric status or wellness participation, such as missed mindfulness sessions. Default thresholds are defined in the GlobalMeasurementThresholds List for psychiatric and wellness scores, including PHQ-9, GAD-7, and mindfulness engagement. These thresholds apply to all patients unless specifically customized by a care manager.

In cases where patient-specific adjustments are needed, Care Managers can modify thresholds to align with a patient's individual psychiatric and lifestyle needs. For example, patients with higher tolerance for psychiatric score variations or those requiring personalized mindfulness goals will have customized thresholds tracked by the system. System 100 may ensure that these custom thresholds are consistently applied across workflows and reflected in reports.

To facilitate timely intervention, system 100 generates alerts when psychiatric or wellness thresholds are exceeded. These alerts are transmitted through instant messaging platforms, ensuring immediate attention from the care team. If critical alerts remain unaddressed within a predefined timeframe, they are automatically escalated to a supervisor or the operations lead. Additionally, system 100 continuously monitors real-time psychiatric and wellness scores and logs alerts directly into the patient's care plan, maintaining ongoing oversight of their health status.

This integration strengthens patient monitoring and proactive care intervention, ensuring that deviations from psychiatric and wellness benchmarks are swiftly identified and addressed.

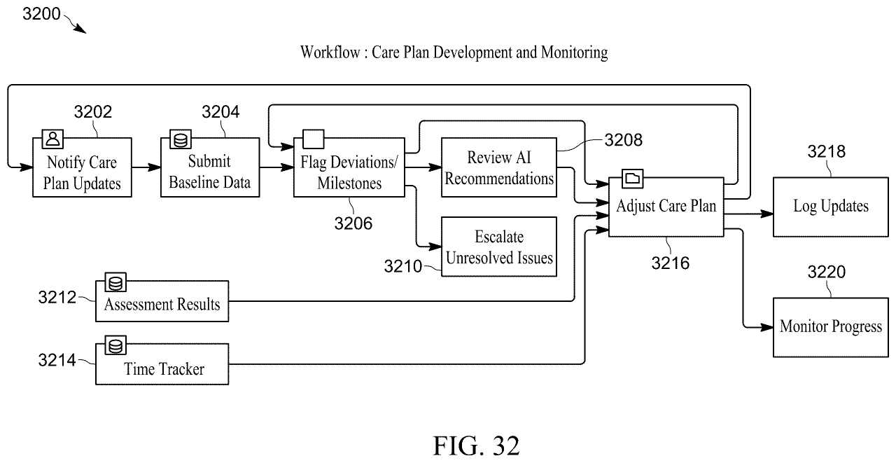

Referring now to FIG. 32, an example workflow diagram 3200 for care plan development and monitoring, depicting the processes for creating, updating, and tracking patient-specific care plans dynamically, is illustrated, in accordance with an embodiment of the present disclosure. This workflow integrates automated notifications, clinician oversight, and patient feedback, ensuring a responsive and adaptive approach to personalized care delivery.

The workflow begins with notifying the relevant care team or patient about updates or initiation of a care plan through a notification module 3202. Notifications may include a summary of the care plan objectives, milestones, and required tasks, ensuring stakeholders are informed of the plan details. The patient or care team then submits baseline data 3204, including assessments, demographic details, and prior health records. These baseline data are used for establishing the initial conditions of the care plan and serve as the foundation for subsequent monitoring and adjustments.

As the care plan progresses, the system monitors milestones and tasks, flagging any deviations or unmet goals through the milestone flagging module 3206. Deviations could include missed sessions, incomplete tasks, or a lack of measurable improvement in patient outcomes. These flagged issues are routed to the review module 3208, where AI-generated recommendations are presented to clinicians. The AI suggestions may include changes to session frequencies, introducing new interventions, or modifying outcome targets based on the identified deviations.

For unresolved or critical deviations, the system automatically triggers an escalation process 3210. This ensures that urgent issues, such as declining patient health metrics or repeated missed appointments, are brought to the attention of senior care managers or specialized clinicians. Escalations are communicated through secure notifications, ensuring timely intervention. Following a review of flagged deviations and AI recommendations, clinicians may adjust the care plan 3216 as necessary. Adjustments could include updating intervention strategies, revising milestones, or altering task deadlines to better align with the patient's progress and needs. These updates are documented in the system using the log updates module 3218, ensuring that changes are recorded for compliance and future reference.

The adjusted care plan is monitored for adherence and effectiveness through the monitoring module 3220. This module integrates data from the assessment results table 3212 and the time tracker 3214 to provide a comprehensive view of patient progress. Metrics such as adherence rates, assessment scores, and task completion timelines are analyzed to determine the effectiveness of the care plan. Real-time dashboards are updated to provide clinicians with clinical insights, enabling continuous monitoring and improvement of care delivery. The workflow includes automated notifications for patients and clinicians to ensure engagement and adherence. For example, patients may receive reminders for upcoming sessions or overdue tasks, while clinicians are alerted about flagged deviations or pending approvals for care plan adjustments. This ensures that parties are consistently aligned with the care plan objectives.

Error handling mechanisms are incorporated into the workflow to address potential issues such as missing data, unresponsive patients, or delays in clinician reviews. The system retries failed notifications and escalate unresolved issues to higher authorities, such as care managers, ensuring that no critical tasks are overlooked. Additionally, all actions and decisions within the workflow are logged in audit trails, providing transparency and supporting compliance with regulatory requirements.

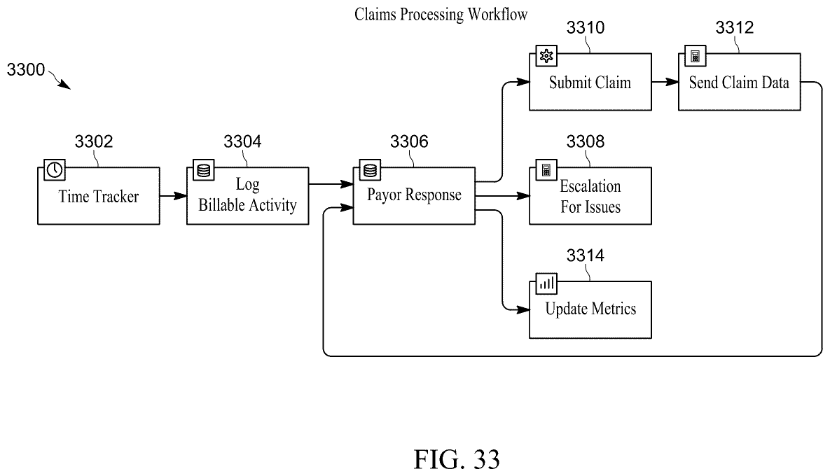

Referring now to FIG. 33, an example flow diagram illustrating a claim processing workflow is provided in accordance with an embodiment of the present disclosure. The claims processing workflow 3300 ensures that billable sessions and tasks are accurately recorded, validated, and submitted while maintaining compliance with payer requirements.

By integrating automated processes with manual interventions, the system enhances efficiency in claims management, reducing errors and ensuring timely reimbursement.

The workflow automates the entire claims lifecycle, covering steps from logging billable activities to handling payer responses, claim rejections, and financial reporting. The primary objectives include ensuring compliance with Current Procedural Terminology (CPT) codes (or other standardized procedure code or code representative of a service event or a line item for a healthcare procedure) and payer policies, automating claim submissions for improved efficiency, handling claim denials with retry and escalation mechanisms, and providing financial insights through reporting tools.

The process begins with time tracking 3302 and logging billable activities 3304, where clinicians record billable tasks or sessions using the timetracker module. The data points logged include CPT codes (such as a code therapy sessions or other standardized procedure code or code representative of a service event or a line item for a healthcare procedure), duration for time-based codes, clinician identification, and service line information (e.g., Psychiatry, Lifestyle Coaching). This ensures that all claimable services are properly documented before submission.

Once the billable data is captured, the system proceeds with payer response handling 3306 by validating claims against CPT code rules (e.g., cross-referencing CPT codes or other standardized procedure code or code representative of a service event or a line item for a healthcare procedure with payer policies), completeness of documentation, and compliance with Medically Unlikely Edits (MUE). If a claim fails validation, it is rejected, and the clinician is notified to correct and resubmit the claim. Validated claims are then automatically submitted to payer systems by the FHIR API for connected payers or through manual export for payers lacking API integration. The claim submission 3310 includes detailed patient information, clinician details, service specifics, and total billable amounts to ensure complete financial transparency.

In cases where claims are rejected or remain pending, the system employs escalation mechanism 3308 to prevent delays in reimbursement. The system retries submission up to three times before escalating the issue for manual review by managers. If errors persist, the billing team is notified for further intervention and resolution. Claims that remain unresolved beyond 30 days are escalated to the department head to ensure priority handling.

To maintain financial oversight, successful claims are logged in the Claims Registry 3312, and real-time financial metrics are updated 3314 in dashboards. This enables stakeholders to track claim success rates, revenue trends by service line, and pending or rejected claims, offering data-driven insights into financial performance.

The system includes robust error handling and escalation logic to minimize claim processing delays. If required claim fields are missing, the system notifies clinicians to complete the entry before submission. For rejected claims, the billing team is alerted to resolve the issue, and for claims that remain pending for more than 30 days, department heads are engaged to expedite resolution.

Several integrations and tools support the seamless execution of this workflow. The timetracker module captures billable clinician activities, while the claims registry tracks the status of each claim. FHIR APIs facilitate direct electronic claim submission to payer systems, and system 100 streamlines claim validation and processing workflows. Additionally, dashboards provide real-time financial insights, ensuring that administrative and financial teams have visibility into claim trends and revenue performance.

In some embodiments, the claims and billing module is designed to automate and streamline the process of generating, tracking, and managing claims for services such as psychiatric evaluations, psychotherapy, and wellness coaching. It ensures that billing is accurate for both insurance and patient payments while also integrating with payroll to manage team compensation. The system 100 automatically assigns billing codes (e.g., CPT codes or other standardized procedure code or code representative of a service event or a line item for a healthcare procedure) to services provided, ensuring compliance with industry standards and payer requirements. Claim generation may be triggered once a service is completed, linking tasks directly to claims to reduce administrative workload. Claims are tracked through various stages, including pending, submitted, paid, and denied, allowing care teams to monitor financial performance effectively. In case of denials, the system alerts billing managers for quick resolution and resubmission.

Additionally, the module incorporates time tracking to ensure proper billing and payroll processing. Using system 100, care team members can log time spent on tasks, with a persistent tracking banner preventing overlap or errors. The system differentiates between billable and non-billable minutes, ensuring that eligible services are charged while still logging all time for payroll purposes. This data seamlessly integrates into payroll processing, where team members are compensated based on logged minutes and predefined pay rates. Payroll files are automatically generated and exported to financial tools for further analysis and/or billing, with administrators able to review and resolve discrepancies before processing payments.

To maintain financial transparency, the system includes real-time payment tracking and reporting using dashboards, which provide insights into total claims submitted, revenue generated, and outstanding payments. The system reconciles billed time with payroll records, ensuring fair compensation and identifying discrepancies between services rendered and services billed. Furthermore, the module is HIPAA-compliant, safeguarding sensitive patient data, and generates audit logs for every claim submission, payment, and payroll file, helping organizations stay compliant with healthcare regulations. The claims and billing module minimizes administrative burden, enhances financial tracking, and ensures accurate billing and payroll management, ultimately improving operational efficiency and compliance within healthcare organizations.

In summary, this automated claims processing workflow significantly enhances efficiency, compliance, and financial tracking by integrating automated submission, validation, escalation, and reporting tools. The combination of automated retries, manual escalation pathways, and real-time data analytics ensures that billing errors are minimized, reimbursement timelines are optimized, and financial oversight is maintained across the healthcare system.

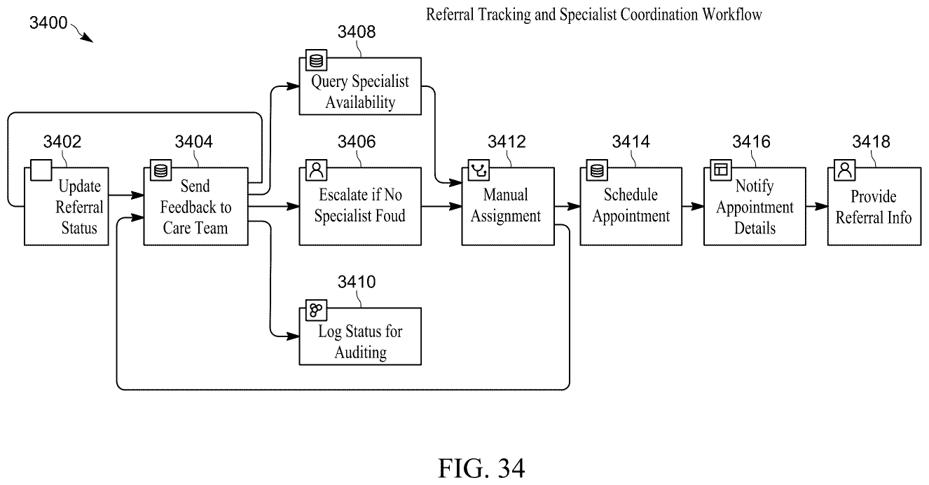

Referring now to FIG. 34, an example flow diagram illustrating a referral tracking and specialist coordination workflow is provided in accordance with an embodiment of the present disclosure. The referral workflow 3400 ensures efficient referral management, enabling seamless coordination between care teams, specialists, and patients. By integrating automated processes with manual intervention mechanisms, the system facilitates timely referral assignments, status updates, and appointment scheduling, thereby enhancing transparency and efficiency in patient care.

The referral workflow 3400 is designed to facilitate referrals to external specialists or within the system while ensuring timely updates on referral status and appointment outcomes. The process is triggered when a care team member identifies the need for a referral, such as in cases where a patient requires specialized consultation. Additionally, automated clinical flags—for example, based on elevated PHQ-9 or GAD-7 scores—can trigger a referral without manual intervention.

The workflow begins with updating the referral status 3402 in the system when a referral is initiated, assigned, or completed. A feedback is forwarded to care team 3404. This ensures that real-time progress tracking is available for both care teams and patients. Once the referral is logged, the system automatically queries available specialists 3408 by matching the referral request with providers in the practitioner registry, the matching process considers parameters such as specialty (e.g., neurology, cardiology, etc.), availability (open time slots), and urgency level (routine vs. urgent referrals).

If no specialist is found, the system initiates an escalation process 3406 by notifying the manager for manual intervention. The manager or assigned personnel can then manually assign 3412 a specialist to ensure that the referral is not left unprocessed. Once a specialist is assigned, the appointment is scheduled 3414. If the referral is directed to an internal specialist, the appointment details are logged in the booking tracker, while for external specialists, the patient receives scheduling details to coordinate their visit independently.

Following the scheduling process, automated notifications are sent to patients 3416, referring clinicians, and care teams through multiple communication channels, including the patient app, email notifications, and instant messaging platforms. The patient is also provided with comprehensive referral details 3418, such as the assigned specialist's contact information, appointment time, and location, ensuring that they have all the necessary information for their consultation.

To maintain the integrity and efficiency of the referral system, robust error handling, and escalation mechanisms are implemented. If a referral is missing essential details, the system flags validation errors and prompts for corrections before submission. If no specialist is available, the issue is escalated for manual assignment. In cases where a referral is not acknowledged within 48 hours, the care coordinator is notified, and if unresolved after 72 hours, the case is escalated to a supervisor for immediate intervention.

The workflow is supported by multiple integrations and automation tools. The referral tracking table logs referral details and status 3410, while the practitioner registry enables real-time specialist availability queries. Bookings tracker stores scheduled appointments, ensuring structured data management. Instant messaging platforms and email notifications facilitate communication with care teams and patients, and example programs such as Microsoft's Power Automate® automates workflow tracking and escalations. Additionally, dashboards provide real-time analytics to monitor referral performance metrics, such as completion rates and pending cases.

The automated referral tracking and specialist coordination workflow enhances efficiency, transparency, and accountability in patient referral management. By combining automated referral handling, real-time tracking, escalation mechanisms, and integrated communication tools, the system ensures that patients are seamlessly connected with specialists, minimizing delays in care delivery while keeping all stakeholders informed.

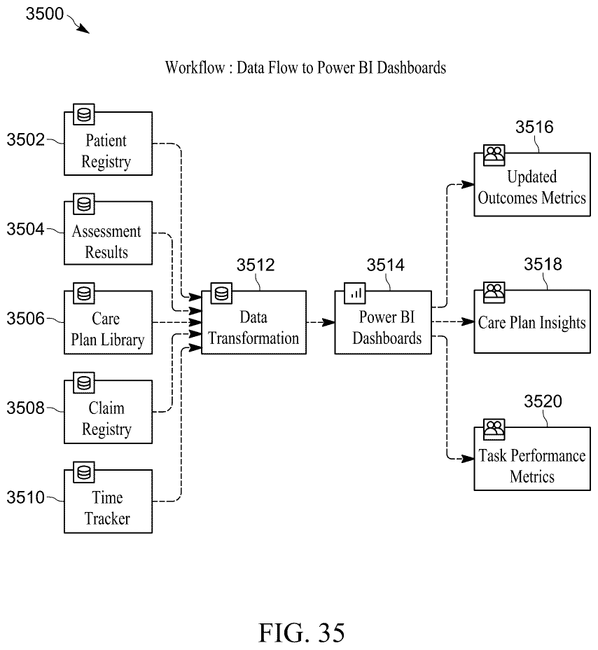

Referring now to FIG. 35, an example workflow diagram 3500 for the seamless data flow into dashboards designed for real-time insights, is illustrated, in accordance with an embodiment of the present disclosure. This workflow consolidates clinical, operational, and administrative data from multiple sources to enable clinical insights into patient outcomes, clinician performance, task efficiency, and financial metrics, including claims processing. By centralizing and transforming data, the system ensures timely and accurate visualization for stakeholders, fostering data-driven decision-making.

The workflow begins with data consolidation from various sources. The patient registry 3502 serves as a repository for patient demographics, service line enrollments, and engagement history. Assessment results 3504 provide longitudinal trends and recent scores from assessments such as PHQ-9 or GAD-7, offering a quantitative measure of patient progress. The care plan library 3506 supplies active and completed care plan data, while the claims registry 3508 contributes financial and billing details, including CPT codes (or other standardized procedure code or code representative of a service event or a line item for a healthcare procedure) and claim statuses. The time tracker 3510 records task completion logs and the allocation of time for both billable and non-billable activities. These collected data form the foundation for the transformation process. Next, the data transformation module 3512 processes and cleanses the raw data to ensure compatibility with dashboard visualizations. The module maps database fields to pre-defined dashboard categories, such as mapping “PHQ-9 Score” to “Assessment Metrics.” Filters may be applied to focus on specific service lines, such as psychiatry, or aggregated timelines, such as weekly trends. This transformation step normalizes the data, preparing it for integration into the reporting system.

Once processed, the transformed data is sent to the dashboard engine 3514, where it is integrated into pre-configured templates. The dashboards automatically refresh to reflect the latest metrics, including patient trends, care plan adherence, task performance, and claims statuses. Updated outcomes metrics 3516 showcase patient recovery rates or trends in assessment scores, while care plan insights 3518 detail adherence rates and progress against established milestones. Task performance metrics 3520 visualize task completion rates, escalation trends, and time allocation efficiency. These dashboards serve as a single source of truth for monitoring and strategic planning. The system ensures proactive notifications to stakeholders about updates or changes in the dashboards. For example, care teams may receive alerts about flagged patient metrics, such as worsening assessment scores or overdue tasks, prompting immediate action. Notifications may also highlight operational metrics, such as an increase in claims rejection rates or task escalation rates exceeding predefined thresholds.

Error handling mechanisms are embedded within the workflow to ensure data accuracy and system reliability. The system logs errors during data retrieval, such as missing fields or failed queries, and attempts retries with incremental delays. If repeated attempts fail, a task is generated for manual reconciliation, and stakeholders are notified of persistent issues. Escalations are triggered for errors, such as discrepancies in claims data or prolonged dashboard synchronization failures, ensuring swift resolution. The decision-making framework within the workflow includes multiple escalation points. For example, if a patient registry lacks care plan updates or if discrepancies are found in task timestamps, the system alerts relevant stakeholders, such as clinicians or administrators, to resolve the issue. Similarly, flagged metrics from dashboards, such as overdue tasks or claims statuses, trigger notifications to ensure prompt action by the appropriate team.

In some embodiments, the system tracks all referral services, including both internal and external referrals to specialists such as nutritionists and sleep specialists. These reports ensure that patient care remains coordinated, and outcomes are linked back to the original care plan. The referral tracking component logs referrals made for psychiatric and lifestyle services, identifying external providers involved and tracking referral statuses such as pending or completed. Additionally, it provides insights into post-referral outcomes, ensuring continuity of care after external services have been utilized.

One or more analysis tools/dashboards (e.g., Microsoft's Power BI®) monitors patient outcomes post-referral, tracking whether the referral resulted in improvement or if additional follow-up is necessary. It also enables comparative data analysis on referral effectiveness across various patient demographics and service lines, allowing for data-driven optimization of referral strategies.

Supervisors and administrative personnel require a high-level view of patient care and operational efficiency. Dashboards aggregate data on care team performance across psychiatric and wellness services, ensuring alignment with Chronic Care Management (CCM) goals and patient outcomes. These dashboards include a timesheet review feature, providing supervisors with insights into how time is allocated across psychiatric and lifestyle care plans. Such dashboards highlight team members who are managing their time efficiently and track clinical and wellness task completion.

An escalation management report is also included, tracking psychiatric and wellness tasks that have been escalated due to delays or importance. This allows supervisors to intervene in cases where team members encounter challenges in task completion. Additionally, overall team productivity metrics aggregate task completion and care plan progress across psychiatric and lifestyle service lines, offering performance comparisons across different care roles such as psychiatrists, health coaches, and psychotherapists.

System 100 facilitates automation across all reporting features, ensuring that psychiatric, lifestyle, and referral data remains synchronized in real-time across patient data and dashboards. This real-time reporting capability ensures that live data is continuously available for patient care, task tracking, and operational performance monitoring.

The enhanced reporting and analytics in the system enable continuous tracking of psychiatric and wellness outcomes, ensuring seamless integration into care plans. By providing insights into patient progress, task efficiency, and team performance, the system enhances holistic care, integrating mindfulness, wellness coaching, and psychiatric care across all service lines.

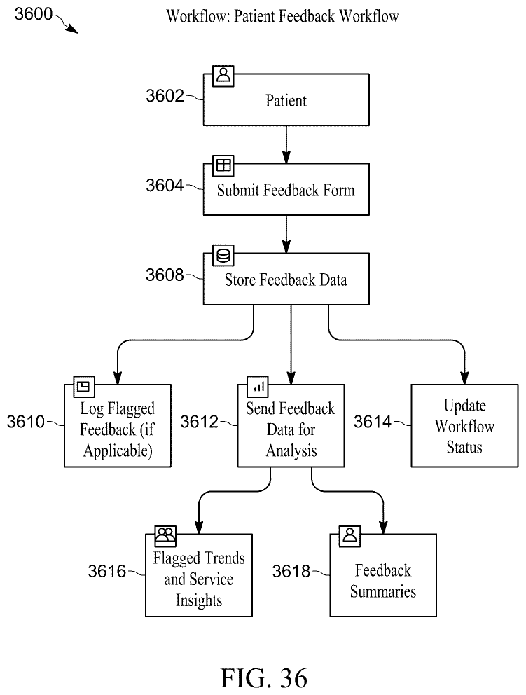

Referring now to FIG. 36, an example workflow 3600 for collecting, processing, and analyzing patient feedback, is illustrated, in accordance with an embodiment of the present disclosure. This workflow ensures the efficient capture of patient experiences following particular interactions, such as therapy sessions or care plan reviews, and enables automated processing, escalation of flagged concerns, and continuous improvement of services.

The workflow including providing to a patient 3602 a feedback form using various channels, such as app notifications or email prompts. Feedback forms 3604 are designed to capture metrics, including satisfaction ratings, open-ended comments, and specific session details. These forms may also include mandatory fields to ensure comprehensive data collection. Upon submission, the feedback data is securely stored 3608 in a feedback registry. This registry links the feedback to relevant patient records, interaction types (e.g., therapy sessions), and submission timestamps for traceability.

The stored feedback undergoes processing to identify clinical insights. In cases of flagged feedback, such as low ratings or negative comments, the system logs the flagged feedback 3610 for prioritized attention. Flagged responses are further categorized based on keywords indicating dissatisfaction or systemic issues, such as delays or unfulfilled expectations. Feedback data, including flagged and non-flagged entries, may also be transmitted 3612 for detailed analysis. The analysis phase generates aggregate trends, identifies recurring issues, and highlights specific areas for service improvement.

The system updates workflow status 3614 in real-time to reflect the completion of feedback collection, validation, and processing. This ensures end-to-end visibility into the feedback management lifecycle. Updated workflow statuses are logged to enable oversight and auditability of actions taken. The analyzed data contributes to flagged trends and service insights 3616, providing high-level visibility into systemic challenges or areas needing immediate intervention. Additionally, feedback summaries 3618 are compiled to offer individual clinicians or care teams concise insights into their interactions, promoting accountability and service enhancement.

The feedback workflow incorporates escalation logic for handling critical cases. For example, if a patient provides a satisfaction rating below a predefined threshold, such as 3 out of 5, the system automatically escalates the feedback to a care coordinator or supervisor. Similarly, flagged comments containing negative keywords trigger notifications to appropriate stakeholders for resolution. Unresolved escalations after a predefined time frame, such as 48 hours, are further escalated to departmental leads for immediate action. Error detection and fallback mechanisms ensure workflow reliability. If mandatory fields in feedback forms are incomplete, the system prompts the patient to resubmit. Errors during data storage, such as connectivity issues, trigger retries, and unresolved failures are logged for administrative review. Patients are notified of submission errors and provided with options to retry, ensuring a seamless experience.

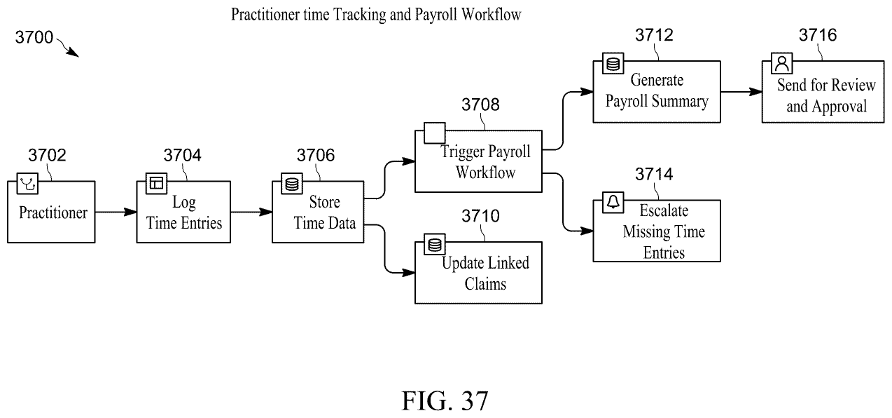

Referring now to FIG. 37, an example flow diagram illustrating a workflow 3700 that automates time tracking for practitioners, ensuring accurate payroll processing while integrating with the claims and billing system, is provided in accordance with an embodiment of the present disclosure. The workflow 3700 further incorporates approval mechanisms and escalations to address errors or missing time entries, ensuring compliance and operational efficiency.

The workflow 3700 is designed to accurately log, categorize, and process practitioner time entries, facilitating seamless integration with payroll and billing systems. It also automates notifications, approvals, and error-handling mechanisms to streamline administrative workflows. The process is triggered either when a practitioner completes a task 3702, such as a therapy session or care plan review, or when a missed time entry is detected, prompting an escalation for resolution.

The process begins with practitioners logging their work hours in the system 3702, distinguishing between billable activities, which involve patient-facing tasks linked to CPT codes or other new or standardized procedure codes or codes representative of a service event or a line item for a healthcare procedure (e.g., therapy sessions), and non-billable activities, such as team meetings or documentation reviews. Once logged, the time data is stored and categorized 3704, with billable activities linked to the claims registry for claim validation and non-billable activities stored in the timetracker for payroll processing.

Upon storing the time entries, the system automatically triggers the payroll workflow 3708, ensuring that all recorded hours are processed for compensation. If any missing time entries are detected 3714, an escalation process is initiated to prompt resolution. The system then updates the linked claims 3710, reconciling billable hours with claims submissions. If discrepancies arise—such as incorrect CPT codes (or other standardized procedure code or code representative of a service event or a line item for a healthcare procedure) or duplicate entries—the system flags the errors for review to prevent billing inconsistencies.

Once the payroll summary is generated 3712, it includes total hours worked, a breakdown of billable vs. non-billable time, and compensation calculations based on predefined rates. If a practitioner fails to log time for a scheduled task, they receive an automated reminder to submit their entries. If no action is taken within 24 hours, the issue is escalated to the care coordinator for further resolution.

Before finalizing payroll processing, managers review payroll summaries and approve or reject entries 3716. If approvals are delayed beyond 48 hours, the system escalates the issue to the finance head, ensuring that payroll processing remains on schedule.

To maintain the accuracy and integrity of time tracking and payroll processing, robust error handling and escalation mechanisms are embedded in the workflow. If time entries are missing, practitioners receive a reminder notification. If unresolved within 24 hours, the issue is escalated to the care coordinator. In cases where payroll discrepancies occur, such as incorrect CPT codes (or other standardized procedure code or code representative of a service event or a line item for a healthcare procedure) or duplicate time logs, the issue is escalated to the billing administrator for resolution. If payroll approvals remain pending beyond 48 hours, the finance head is notified to intervene.

The workflow leverages multiple tools and integrations to automate and streamline time tracking and payroll processing. The clinician app enables practitioners to log time entries, while the timetracker table stores all logged time data for reporting and categorization. The Claims Registry ensures that billable hours are accurately linked to claims, preventing discrepancies. Payroll summary Table maintains payroll reports for review and approval, and system 100 facilitates automation of time tracking, payroll processing, and approval workflows. Instant messaging notifications and email notifications ensure that practitioners, managers, and finance teams receive timely updates and alerts.

The automated payroll workflow enhances accuracy, compliance, and efficiency by reducing manual errors, ensuring proper categorization of work hours, and integrating seamlessly with billing and payroll systems. The incorporation of escalation mechanisms ensures that missing entries, billing discrepancies, and delayed approvals are promptly addressed, thereby improving transparency, practitioner accountability, and financial management.

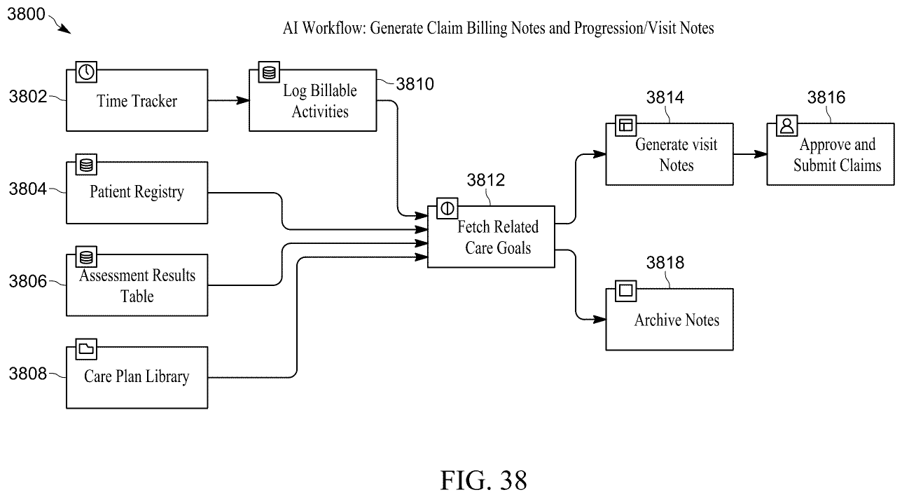

Referring now to FIG. 38, an example flow diagram illustrating a process for generating claims billing notes and progress visit notes is provided in accordance with an embodiment of the present disclosure. The workflow 3800 automates the generation of billing notes and visit summaries, ensuring accurate claim submission and streamlined care documentation.

The process facilitates automated documentation of billable activities, retrieval of patient care data, AI-driven generation of billing and progress notes, and seamless claim submission. This workflow minimizes administrative workload, enhances accuracy, and ensures compliance with standardized documentation practices.

The workflow is initiated when practitioners log billable tasks 3810 into the Timetracker 3802, such as therapy sessions or medical consultations. This logging process ensures that all services rendered are accurately recorded for subsequent claim generation. The system then fetches related care goals 3812 and patient data from multiple sources, including the patient registry 3804 for demographics and history, the assessment results table 3806 for test scores and evaluations, and the care plan library 3808 for long-term treatment objectives. These retrieved data points serve as inputs for generating personalized billing and progress notes.

The AI-driven claim billing note generation process utilizes multiple data sources, including session duration from timetracker, CPT codes (or other standardized procedure code or code representative of a service event or a line item for a healthcare procedure) from claims registry, and practitioner notes detailing session observations. For instance, the AI automatically constructs billing notes, such as claim billing note: “45-minute CBT session for depression, CPT Code XXXX. Practitioner: Dr. Jane Doe.”

Following billing note creation, the AI compiles progress and visit notes 3814, summarizing patient progress based on care plan milestones, practitioner observations, and assessment results. These notes provide a comprehensive clinical summary of the patient's condition and ongoing treatment progress. For example Progress Note: “Patient reports improved mood and decreased anxiety. Next steps: Introduce the journaling task.”

Once billing and progress notes are generated, they undergo a review process for accuracy and completeness. Upon approval, claims are submitted for processing 3816, ensuring that all billable services are properly documented and compliant with reimbursement requirements.

The final stage of the workflow involves archiving billing and progress notes 3818 for compliance and record-keeping purposes. This ensures that historical patient and billing data are maintained for auditing, reporting, and regulatory compliance.

By integrating automated documentation, AI-powered content generation, and claim submission workflows, this process reduces administrative burden, ensures standardized documentation, and expedites claim approval. The workflow enhances operational efficiency, minimizes errors, and facilitates seamless financial and clinical documentation processes, ultimately improving patient care coordination and reimbursement accuracy.

In some embodiments, the document automation and management system streamlines the creation, organization, and accessibility of patient-related records, ensuring that psychiatric progress notes, lifestyle evaluations, and mindfulness progress reports are automatically generated and stored in a centralized repository. This eliminates the need for manual documentation and enhances efficiency in patient care management. The system 100 uses predefined templates for psychiatric and wellness assessments, such as mindfulness evaluations and psychiatric progress notes, which ensures consistency and standardization across all patient records. These templates are automatically populated with patient-specific data and stored in the designated teams channel or an equivalent document management system.

To further enhance workflow efficiency, the system integrates with form/list software, allowing health coaches and mindfulness instructors to track wellness progress and mindfulness outcomes. The data collected through these forms is automatically converted into structured reports and linked directly to the patient's care plan, providing real-time visibility to the care team. Additionally, automated workflows trigger document generation and storage immediately upon completion of assessments, ensuring that wellness-related records, such as mindfulness session summaries, are readily available without the need for manual intervention.

Another feature of the system is the real-time alerting mechanism. If an assessment reveals significant deviations in psychiatric or wellness metrics, such as a high PHQ-9 score indicating severe depression or a decline in mindfulness engagement, the system generates an alert and notifies the care manager. This proactive approach allows for timely intervention, ensuring that potential health concerns are addressed before they escalate. By automating documentation, integrating assessment tracking, and enabling real-time alerts, the system improves patient care coordination, reduces administrative workload, and enhances the overall efficiency of psychiatric and wellness care.

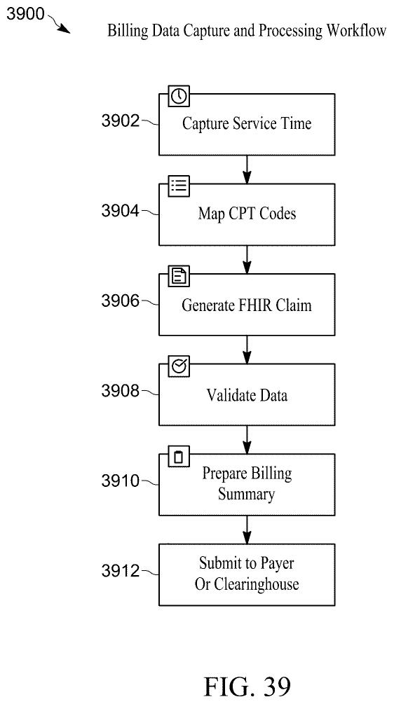

Referring now to FIG. 39, an example flow diagram illustrating the process of capturing, validating, and submitting billing data to ensure accurate claim generation and seamless payer integration is depicted in accordance with an embodiment of the present disclosure. The workflow 3900 automates the billing data capture process, maps appropriate CPT codes (or other standardized procedure code or code representative of a service event or a line item for a healthcare procedure), generates FHIR-compliant claims, validates claim data, prepares billing summaries, and submits claims to payers or clearinghouses. This automation optimizes billing accuracy and enhances reimbursement efficiency while ensuring compliance with payer regulations.

The process begins with the capture of service time 3902, where clinicians record the duration of services provided. The system tracks this time in real-time for all billable activities, ensuring accurate logging of session durations. Once the service time is captured, the system assigns the appropriate CPT code 3904 (or other standardized procedure code or code representative of a service event or a line item for a healthcare procedure) based on service type, duration, and billing rules. For example, if a therapy session lasts for 90 minutes, the system automatically maps it to a CPT Code (or other standardized procedure code or code representative of a service event or a line item for a healthcare procedure), utilizing two add-on codes of 30 minutes each. This ensures that billing is correctly structured according to payer policies, reducing errors and potential denials.

Once the CPT codes (or other standardized procedure code or code representative of a service event or a line item for a healthcare procedure) are mapped 3904, the system proceeds to generate a FHIR-compliant claim 3906. Captured billing data is converted into standardized FHIR resources to maintain interoperability with payer systems. Several FHIR resources are utilized in this process. The patient resource stores demographic information, ensuring that claims are correctly linked to individuals receiving care. The service request resource documents session details, including the nature of the service and its duration. The practitioner resource links the provided service to the respective healthcare provider, ensuring accurate attribution of the claim.

Before submission, the system validates the claim data 3908 by applying predefined validation rules to ensure accuracy and compliance with payer requirements. This validation process confirms that CPT codes (or other standardized procedure code or code representative of a service event or a line item for a healthcare procedure) conform to payer-defined billing limits and guidelines, ensuring that claims are not flagged for errors. Additionally, all mandatory claim fields, including provider and patient details, must be complete before submission. The system also leverages FHIR validation tools to confirm adherence to industry standards, ensuring that claims are correctly formatted and interoperable with payer systems.

After successful validation, the system prepares a billing summary 3910, organizing the billing information based on multiple parameters. The summary categorizes claims by service type, such as therapy, medical consultations, or diagnostic services. It also consolidates patient encounters to streamline claim submissions and prevent duplicate entries. Additionally, the billing summary accounts for payer contract rates, ensuring that claims are categorized and priced according to the agreements with different insurance providers.

In the final step, the system submits the finalized billing data to the payer or clearinghouse in FHIR-compatible batches 3912. This structured submission process enables fast claim processing and reduces the likelihood of denials due to formatting or compliance errors. The use of FHIR APIs ensures seamless integration with payer systems, facilitating real-time data synchronization and improving the overall accuracy of reimbursements.

By leveraging FHIR standards, automated validation mechanisms, and optimized billing workflows, this system enhances claim submission efficiency, minimizes administrative errors, and improves financial outcomes for healthcare providers. The automation of time tracking, CPT code mapping (or mapping of other standardized procedure code or code representative of a service event or a line item for a healthcare procedure), claim validation, and payer submission significantly reduces the manual workload while ensuring that claims are processed swiftly and accurately.

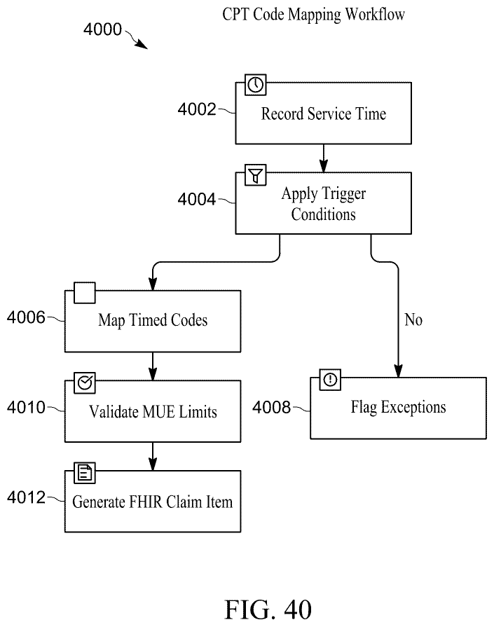

Referring now to FIG. 40, an example flow diagram illustrating a workflow 4000 that automates the assignment of CPT codes (or other standardized procedure code or code representative of a service event or a line item for a healthcare procedure) to healthcare services, ensuring compliance with billing regulations and seamless claim processing, is depicted in accordance with an embodiment of the present disclosure. The system optimizes the assignment of CPT codes (or other standardized procedure code or code representative of a service event or a line item for a healthcare procedure) by recording service time 4002, applying predefined trigger conditions 4004, mapping time-based codes 4006, validating payer-imposed limits 4010, flagging exceptions 4008, and generating standardized FHIR claim items 4012. By automating these processes, the workflow ensures accurate billing and regulatory compliance while minimizing manual intervention.

The process begins with recording service time 4002, where clinicians log the duration of services provided. The system captures and stores this data in real-time, ensuring that billable activities are properly documented. This step enables determination of the appropriate CPT code assignment (or assignment of other standardized procedure code or code representative of a service event or a line item for a healthcare procedure) based on service type and duration.

Next, the system applies trigger condition 4004 to determine whether a specific CPT code should be assigned to a service. Each CPT code (or other standardized procedure code or code representative of a service event or a line item for a healthcare procedure) is associated with predefined conditions that dictate its applicability. For example, a code representing Health Behavior Assessment may be automatically triggered when a patient completes an assessment form and the care team finalizes the preliminary analysis report. These conditions ensure that valid and medically necessary services are billed, reducing errors and claim rejections.

The system then proceeds to map timed code 4006, where service durations are aligned with the appropriate CPT codes (or other standardized procedure code or code representative of a service event or a line item for a healthcare procedure). Timed codes, such as those used in psychological testing, are assigned based on the length of the session. For example, a 90-minute psychological test would be mapped as follows: CPT Code A (Base Code, 30 minutes)=1 unit and CPT Code B (Add-on Code, 30 minutes)=2 units. Non-timed codes, such as CPT Code C, are assigned based on a single instance of the service, regardless of duration. This mapping ensures that claims accurately reflect the services rendered while adhering to payer policies.

To maintain compliance with billing regulations, the system validates MUE (Medically Unlikely Edits) limits imposed by payers 4010. This validation process ensures that CPT codes do not exceed predefined unit thresholds. For example, CPT Code B allows a maximum of 11 units per day, and any additional units beyond this limit are automatically flagged and excluded from claim submission. This step prevents overbilling and ensures that claims remain within payer-approved limits.

If a service does not meet the necessary billing criteria, the system flags exceptions 4008 for manual review. For example, non-billable activities such as administrative tasks may be logged in the system but are assigned dummy codes for tracking purposes instead of being submitted for reimbursement. This flagging mechanism allows billing administrators to review and resolve discrepancies before claim submission, ensuring billing accuracy.

In the final step, the system generates an FHIR claim item 4012, embedding the finalized CPT codes (or other standardized procedure code or code representative of a service event or a line item for a healthcare procedure) into FHIR-compliant claims (or other predefined claim compliance rules) for seamless submission to payers. This ensures that all billing records adhere to standardized, interoperable formats, allowing for efficient claim processing and integration with payer systems. The use of FHIR APIs enhances data consistency and facilitates real-time synchronization with billing platforms.

By automating CPT code (or other code) assignment and validation, this workflow minimizes administrative burden, improves claim accuracy, and enhances compliance with payer regulations. The structured approach ensures that all billable services are appropriately coded, medically necessary, and within regulatory limits, ultimately optimizing revenue cycle management within the automated billing system.

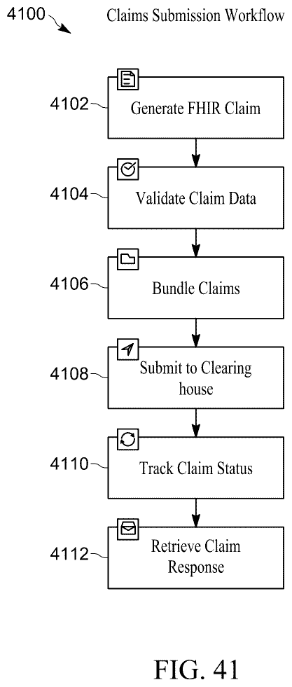

Referring now to FIG. 41, an example flow diagram illustrating an automated claim submission and tracking process is depicted in accordance with an embodiment of the present disclosure. The workflow 4100 ensures efficient integration with payers and clearinghouses, facilitating seamless reimbursement processing through automation, validation, and real-time tracking mechanisms.

The process begins with generating an FHIR claim 4102, wherein the system processes billing data to create an FHIR-compliant claim. Each generated claim includes elements such as CPT codes (or other standardized procedure code or code representative of a service event or a line item for a healthcare procedure), service details, and patient information, ensuring structured and standardized claim submission. By utilizing FHIR (Fast Healthcare Interoperability Resources) APIs, the system enhances interoperability and ensures seamless exchange of billing information with external payer systems.

Following claim generation, the system performs claim data validation 4104 to minimize rejections and ensure accuracy before submission. The validation process includes multiple checks, such as matching CPT codes (or other standardized procedure code or code representative of a service event or a line item for a healthcare procedure) with service duration, verifying patient insurance coverage, and ensuring the presence of all required fields. If any discrepancies are detected, such as missing insurance details, the claim is flagged for correction before proceeding to submission. This proactive approach reduces claim denials and enhances processing efficiency.

Once validated, claims are bundled into FHIR-compatible batches 4106 to optimize submission efficiency. Grouping multiple claims into batches streamlines processing, reduces submission overhead, and accelerates reimbursement cycles. For instance, claims for multiple payers can be consolidated into a single submission, reducing administrative workload and improving claim turnaround times.

The bundled claims are then submitted to a clearinghouse 4108, which acts as an intermediary between the system and multiple payers. The clearinghouse enhances claim processing efficiency by handling multi-payer integration, providing real-time error feedback, and facilitating faster claim adjudication. By leveraging clearinghouse services, the system ensures that claims are submitted in compliance with payer-specific requirements, thereby reducing the risk of rejections and delays.

After submission, the system actively tracks the status of each claim using the FHIR claim response resource 4110. Possible claim statuses include approved (accepted for payment), rejected (requiring manual correction), or pending (under review by the payer). This real-time tracking mechanism enables billing administrators to monitor claim progress, identify issues promptly, and take corrective action if necessary.

Upon receiving the final claim response, the system records and processes the outcome 4112. If the claim is approved, it moves forward to payment processing, ensuring timely reimbursement. If the claim is rejected, it is flagged for resubmission with the necessary corrections, allowing for efficient issue resolution. For example, if a psychologist submits a claim for CPT Code D (Cognitive Assessment), the initial response may indicate that the claim is pending review. Once processed, the final response may indicate approval, along with the associated Explanation of Benefits (EOB).

The workflow automates and streamlines the claims submission and tracking process, ensuring error-free claim submissions, faster reimbursements, and compliance with payer regulations. By leveraging FHIR-based interoperability, automated validation checks, and real-time claim tracking, the billing system enhances operational efficiency and optimizes revenue cycle management.

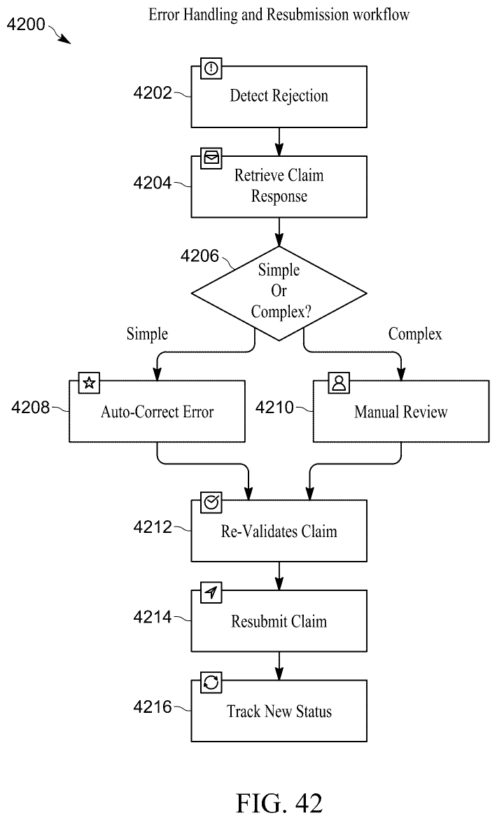

Referring now to FIG. 42, an example flow diagram illustrating an automated error handling and resubmission process for claim rejections is depicted in accordance with an embodiment of the present disclosure. The workflow 4200 ensures efficient identification, correction, and resubmission of rejected claims while maintaining compliance with payer regulations and optimizing reimbursement efficiency.

The process begins with detecting rejected claims 4202, wherein the system leverages the FHIR claim response resource to identify claims that have been denied by payers. The rejection may occur due to various reasons, including data inconsistencies (e.g., missing patient details), CPT code (or other code) mismatches (e.g., exceeding Medically Unlikely Edit (MUE) limits), coverage limitations (e.g., non-covered services), or technical issues (e.g., incorrect formatting in the FHIR claim submission). By automating the detection of claim denials, the system ensures that errors are promptly addressed.

Following rejection detection, the system proceeds to retrieve the claim response from the payer 4204, which contains status updates, error messages, and specific reasons for rejection. This detailed feedback allows the system to determine the necessary corrective actions. For example, if a claim is rejected due to an invalid policy number, the system flags the issue for resolution.

Next, the system performs an error complexity analysis to classify the issue as either a simple error (automatically correctable) or a complex error (requiring manual intervention) 4206. Simple errors, such as minor formatting mistakes or missing patient details, can be auto-corrected by the system 4208. In contrast, complex errors, such as CPT code/code mismatches, exceeding billing limitations, or policy-related denials, require manual review by billing specialists 4210. For example, if a psychologist submits 12 units for a psychological test instead of the allowed 11, the billing team may be triggered by the system 100 to manually adjust the claim to comply with the MUE limit.

Once errors are identified, the system moves to the error correction phase, wherein simple errors are automatically rectified by updating missing fields, correcting formatting errors, or adjusting minor discrepancies. In contrast, complex errors undergo manual review, ensuring that payer-specific requirements and policy constraints are correctly addressed. This structured correction approach minimizes resubmission delays and enhances claim acceptance rates.

After correction, the claim undergoes re-validation 4212, where the system performs a secondary verification to ensure that all required data fields are complete, payer-specific rules are met, and FHIR schema validation is maintained for proper formatting. This additional validation step ensures that the resubmitted claim has a higher likelihood of approval.

Following successful validation, the corrected claim is resubmitted to the clearinghouse, which processes the claim for payer review 4214. The resubmission may result in two possible outcomes:

Approved—The claim is accepted, allowing it to proceed to payment processing.

Rejected Again—If additional corrections are required, the process is repeated, and further manual review may be necessary.

Throughout this process, the system actively tracks the status of the resubmitted claim, ensuring real-time monitoring of payer feedback 4216. If the claim is approved, it advances to the reimbursement stage. If the claim is rejected again, the system flags it for further review, and additional corrective actions are taken as needed.

For example, consider a scenario where a claim for a psychological test is rejected due to exceeding the MUE limit. The system automatically detects the rejection using the claim response resource, and the billing team reviews the error. Upon determining that 12 units were submitted instead of the allowed 11, the claim is corrected, validated, and resubmitted with the correct MUE limit. Upon review, the payer approves the corrected claim, ensuring successful reimbursement.

The automated and structured resubmission process significantly minimizes claim rejections, optimizes reimbursement efficiency, and ensures compliance with payer rules. By integrating FHIR-based claim tracking, automated error detection, and intelligent correction mechanisms, the system enhances revenue cycle management, reducing administrative burden while improving financial outcomes for healthcare providers.

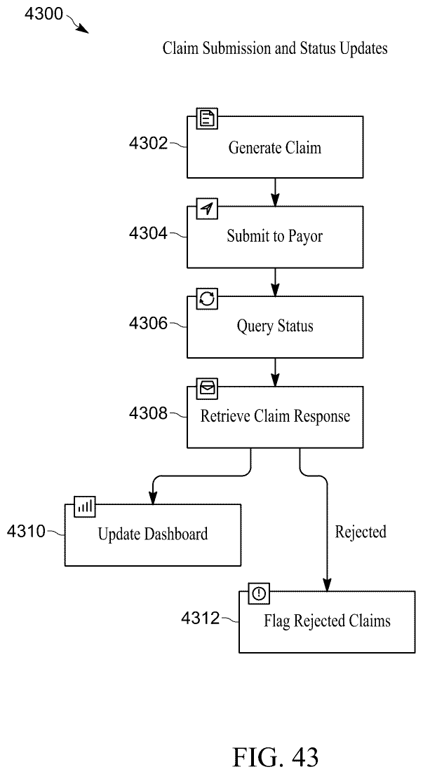

Referring now to FIG. 43, an example flow diagram illustrating the automated claim submission and status update process is depicted in accordance with an embodiment of the present disclosure. The workflow 4300 ensures seamless submission, real-time tracking, and efficient management of claims, thereby optimizing reimbursement cycles and reducing administrative overhead.

The process begins with claim generation 4302, wherein billing data is processed and converted into an FHIR claim. This ensures that the claim includes all necessary information, such as patient demographics, provider details, service codes, and billing classifications, while also ensuring compliance with payer-specific formatting and submission requirements. By utilizing the FHIR standard, the system enhances interoperability and standardization, facilitating smoother transactions between healthcare providers and payers.

Once the claim is generated, it is submitted to the payer or clearinghouse for processing 4304. The FHIR claim is transmitted directly to insurance companies (payers) or clearinghouses, which act as intermediaries for multi-payer integration. For instance, a psychologist submitting a claim for CPT Code D (Cognitive Assessment) can do so through this automated process, ensuring that all required data is properly structured and transmitted for swift review.

Following submission, the system initiates a claim status query 4306, periodically checking the status of the submitted claim by querying the FHIR server. This enables real-time tracking of claim progress and ensures that providers are promptly informed of their claim's status. The system categorizes claims into three possible states:
Pending—The claim is under review by the payer.
Approved—The claim has been successfully processed and is ready for invoicing and payment.
Rejected—The claim has been denied and requires correction before resubmission.

To facilitate further processing, the system retrieves the FHIR claim response resource 4308, which contains detailed feedback from the payer regarding the claim status. This resource provides insights into whether the claim has been approved, rejected, or remains pending for additional review. In cases where a claim is rejected, the claim response resource provides specific error messages, allowing the billing team to identify and correct issues efficiently.

Subsequently, the claim status is updated on the system dashboard 4310, providing billing teams with real-time visibility into claim progress. The dashboard enables efficient monitoring, ensuring that approved claims proceed to invoicing workflows, while rejected claims are flagged for immediate review and correction. This centralized tracking system reduces administrative burden and ensures that billing teams can manage claims more effectively.

If a claim is rejected, the system automatically flags it for correction 4312, leveraging the error details from the claim response resource to provide actionable insights for resolution. For instance, if a claim is rejected due to a missing policy number, the system immediately alerts the billing team, prompting them to update the claim and resubmit it. This proactive approach reduces claim denials and improves reimbursement rates.

The automated claim submission and tracking workflow ensures efficient claim processing, minimizes errors, and accelerates reimbursements. By integrating FHIR-based claim generation, real-time tracking, and intelligent error handling, the system streamlines revenue cycle management, enabling healthcare providers to reduce administrative workload, enhance billing accuracy, and optimize financial performance.

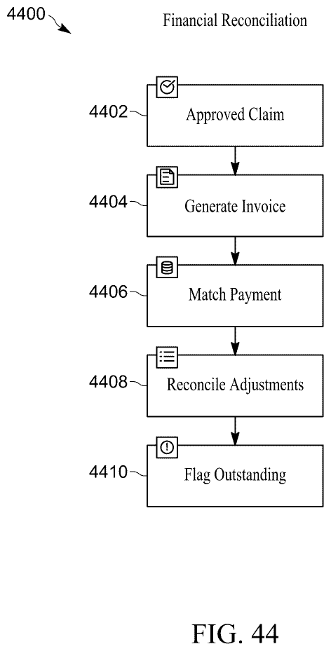

Referring now to FIG. 44, an example flow diagram illustrating the financial reconciliation workflow is depicted in accordance with an embodiment of the present disclosure. Thee workflow 4400 ensures that approved claims are accurately processed, invoices are generated, payments are reconciled, and outstanding balances are efficiently managed, thereby optimizing the revenue cycle and ensuring financial accuracy.

Upon claim approval 4402, the system transitions the claim into the financial reconciliation process. This step ensures that the payer has successfully processed the claim and that it is ready for invoicing without requiring further corrections or resubmissions. The approved claim data serves as the foundation for subsequent financial transactions, ensuring that validated claims proceed to billing and revenue processing.

Once a claim is approved, the system generates an invoice 4404 based on the approved claim details. The invoice contains essential billing information, including the billed amount, expected payment due date, and payer details. This structured invoicing approach ensures clear and accurate financial records, reducing errors in payment processing. Additionally, all invoices are stored in Dataverse, enabling centralized tracking, auditing, and financial reporting.

Following invoice generation, the system matches payments 4406 received from payers with corresponding invoices. This reconciliation process involves reviewing the Explanation of Benefits (EOB) to validate payment details, applying any adjustments (such as credits, refunds, or partial payments), and ensuring that invoice amounts align with received payments. The system 100 may automate this matching process, minimizing manual errors and improving efficiency in financial operations.

If discrepancies arise, the system reconciles adjustments 4408 to address underpayments, overpayments, or denied claims. Underpayments are flagged for follow-up, ensuring that providers receive the correct reimbursement amount. Overpayments are identified, and in some examples, refunds or balance adjustments are processed. Additionally, in cases where payments are reduced due to denials or deductions, the system reviews the reasons for adjustment, providing transparency and enabling appropriate resolution measures.

In cases where payments do not fully cover the invoiced amount, the system 100 flags outstanding balances 4410 and triggers automated alerts for necessary follow-up actions. These actions may include sending reminders to payers for unpaid amounts, escalating overdue invoices for further review, or coordinating with collections teams if required. By automating outstanding balance tracking and follow-up, the system ensures that unresolved financial issues are promptly addressed, reducing revenue leakage and improving cash flow management.

The financial reconciliation workflow provides a structured, automated approach to claim approval, invoicing, payment matching, and outstanding balance resolution. By leveraging data validation, automated payment reconciliation, and proactive issue flagging, this workflow enhances financial accuracy, optimizes revenue cycles, and ensures compliance with payer reimbursement policies within the billing system.

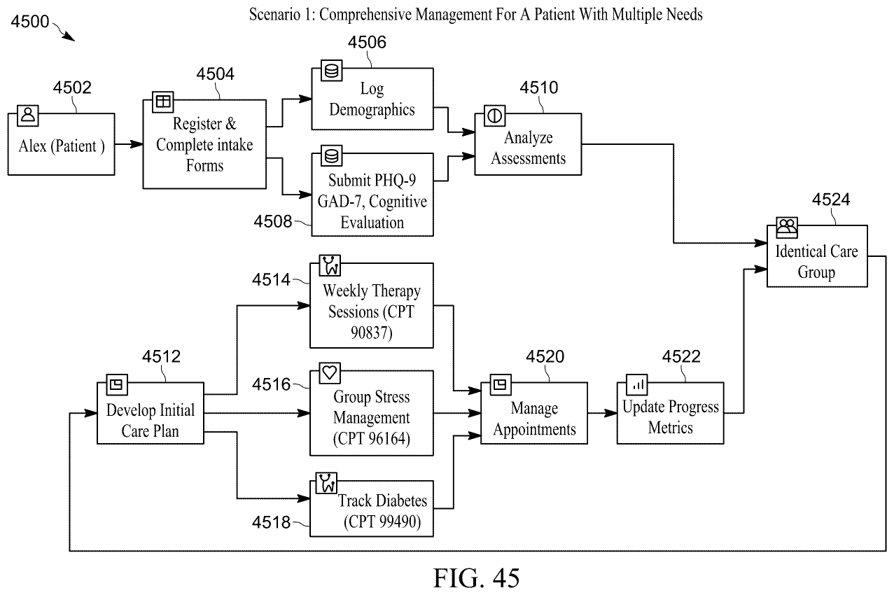

Referring now to FIG. 45, an example use case scenario 4500 for the comprehensive management of a patient, is illustrated, in accordance with an example embodiment of the present disclosure. Alex (i.e., a patient), has multiple medical and mental health needs, including severe anxiety, Type 2 diabetes, and cognitive impairment. This workflow demonstrates the seamless integration of multiple service lines, AI-powered recommendations, and coordinated care to ensure holistic management of Alex's conditions.

The workflow includes receiving completed intake forms 4504 from Alex 4502 through a patient-facing interface. These forms collect, for example, health history, demographics, and preliminary details, which are then logged into the log demographics system 4506. The process may include receiving a series of completed assessments 4508, including PHQ-9, GAD-7, and a cognitive evaluation. The assessment results, such as a high GAD-7 score of 19, are analyzed 4510, triggering notifications for necessary interventions, such as a psychiatric evaluation and enrollment in Chronic Care Management (CCM).

Based on the collected data and assessments, an initial care plan is created 4512. This care plan outlines specific interventions, such as weekly therapy sessions for anxiety 4514 (CPT 90837), group stress management sessions 4516 (CPT 96164), and diabetes monitoring and education 4518 (CPT 99490). The care plan is tailored to address Alex's physical and mental health needs holistically, combining psychiatric services, lifestyle interventions, and chronic care management. Throughout the process, appointments are managed 4520 to ensure Alex has access to scheduled sessions and evaluations. Progress metrics are updated 4522 in real-time to monitor his outcomes across various parameters, such as anxiety improvement and diabetes control. The system utilizes these metrics to identify trends, such as plateauing improvements and recommends adjustments to the care plan. For example, AI-powered analysis suggests increasing therapy frequency and incorporating family counseling to better address Alex's anxiety. The workflow integrates Alex into an identical care group 4524 to provide peer support and shared experiences, which are beneficial for managing his stress and anxiety. Updates to his care plan and interventions are logged and monitored continuously to ensure that his treatment remains adaptive and responsive to his evolving needs.

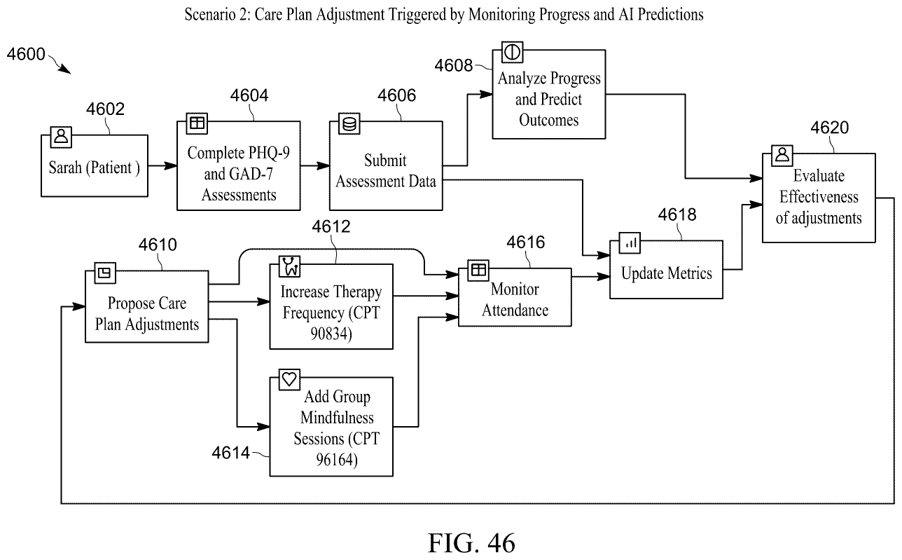

Referring now to FIG. 46, an example scenario 4600, highlighting a care plan adjustment process triggered by monitoring patient progress and AI-based predictions, is illustrated, in accordance with an example embodiment of the preset disclosure. This scenario focuses on Sarah, a patient whose progress stagnates, necessitating a reassessment and tailored modifications to her care plan to achieve better outcomes. The workflow integrates patient assessments, AI-generated recommendations, and care team interventions, ensuring seamless tracking and evaluation.

The process includes receiving completed PHQ-9 and GAD-7 assessments 4604 from Sarah 4602, which are integral to tracking her mental health progress. These assessments are submitted 4606 using a patient-facing interface, and the results are logged into the system. The data undergoes a thorough analysis 4608, where the system evaluates Sarah's progress and predicts outcomes based on historical data, current metrics, and predictive analytics models. Based on the analysis, the system identifies the need for adjustments due to stagnation or lack of significant improvement. The care plan adjustment process 4610 is initiated, where the AI system suggests actionable modifications, such as increasing the frequency of therapy sessions 4612 (CPT 90834) and incorporating group mindfulness sessions 4614 (CPT 96164). These adjustments are tailored to Sarah's specific needs and historical patterns of responsiveness to interventions.

Once the proposed adjustments are approved, implementation is closely monitored 4616 to ensure Sarah attends the newly scheduled sessions and adheres to her updated care plan. Attendance and engagement data are logged and tracked in real-time, providing the care team with immediate visibility into her compliance and progress. The system continuously updates metrics 4618 related to Sarah's progress, which are visualized through dashboards for both care teams and administrators. This feedback loop enables timely evaluations of the effectiveness of the adjustments. If necessary, further modifications are made to the care plan to optimize outcomes. Finally, the effectiveness of the adjustments is thoroughly evaluated 4620. This involves comparing updated metrics with initial baselines to measure improvements and identify remaining challenges. The evaluation ensures that Sarah's care remains dynamic and responsive to her evolving needs.

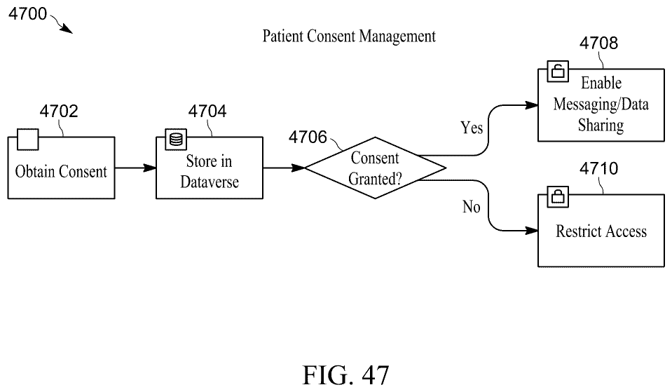

Referring now to FIG. 47, an example flow diagram illustrating the patient consent management workflow is depicted in accordance with an embodiment of the present disclosure. The workflow 4700 ensures that patient privacy preferences are accurately recorded, enforced, and managed, thereby enabling compliance with regulatory requirements such as HIPAA and GDPR while allowing patients to control how their health data is shared.

In the initial step, the system prompts patients to review and provide consent regarding the sharing of their health data 4702. This consent can be obtained through multiple mechanisms, including privacy settings within the patient app, consent forms during patient registration, or explicit requests for third-party data sharing. By offering patients the ability to opt in or out of sharing their health information, the system ensures that data access aligns with patient preferences and regulatory obligations.

Once a patient makes a consent decision, the system securely records the consent status in dataverse 4704. This storage mechanism ensures that all consent records are maintained in real-time, allowing the system to track and enforce patient preferences dynamically. Any updates to the consent status are immediately reflected within the system, ensuring that data access permissions remain up to date and aligned with patient-authorized usage.

Before allowing data access, the system verifies the stored consent status 4706. If consent is granted, the system enables data sharing and messaging with authorized third-party payers, allowing secure API access for retrieving patient data in compliance with the patient's authorization 4708. Conversely, if consent is denied, the system restricts data access to external entities, ensuring that third-party payers cannot retrieve patient information 4710. In such cases, API access is automatically disabled, and any unauthorized access attempts are blocked and logged for security tracking.

For example, if a patient logs into the patient app and opts out of sharing their data with third-party payers, the system updates their consent record in dataverse. Any future attempts by external entities to access the patient's data are subsequently blocked and logged, ensuring that patient privacy settings are strictly enforced.

The patient consent management workflow provides a secure, transparent, and regulatory-compliant method for handling patient data-sharing preferences. By integrating real-time consent tracking, automated enforcement, and security logging, this workflow enhances patient autonomy, prevents unauthorized data access, and ensures adherence to data protection regulations within the healthcare ecosystem.

In some embodiments, the system 100 includes a workflow management and optimization module designed to enhance the efficiency of care teams by tracking their performance, distributing tasks effectively, and supporting their professional development. By leveraging advanced analytics and automation, this module ensures that care teams maintain high standards of patient care while optimizing productivity.

One example aspect of this module is care team performance dashboards, which provide real-time insights into team efficiency and workload distribution. Through dashboards, administrators can monitor metrics such as time spent on patient tasks, task completion rates, and patient caseloads per team member. These insights help ensure a fair distribution of work, prevent burnout, and identify areas for improvement. The system also tracks key performance indicators (KPIs), such as time per task, patient satisfaction scores, and task escalation frequency, allowing for data-driven performance evaluations and comparisons across different teams and service lines.

Task Distribution and Escalations is another essential feature, ensuring that workloads are assigned fairly and overdue tasks are promptly addressed. Using system 100, tasks are automatically allocated based on team members' roles, availability, and workload. For example, psychiatrists are assigned psychiatric evaluations, while health coaches handle wellness interventions. If tasks remain incomplete or become overdue, the system triggers escalation workflows to reassign or prioritize them, ensuring that critical patient care activities are never delayed.

To further optimize workforce efficiency, the system includes workforce optimization through time tracking, which logs time spent on each patient-related task. The timetracker ensures that all time is accounted for-whether billable or non-billable-allowing administrators to analyze workflow efficiency. Dashboards highlight trends in task completion times and idle periods, enabling care managers to refine scheduling and reduce downtime.

Training and professional development is another example component, ensuring that care teams remain compliant with certifications and receive ongoing education. The system tracks certifications and licenses, sending automated alerts when renewals or training are needed. Additionally, it provides role-specific training pathways, ensuring that psychiatrists, health coaches, and other professionals receive targeted learning opportunities. Dashboards monitor training progress, helping administrators support team members in meeting their professional development goals.

Lastly, the module integrates compensation and workforce productivity alignment, linking productivity data with payroll to ensure fair and efficient compensation. Time-tracking data determines productivity-based pay, with system 100 generating payroll files that integrate with accounting software like QuickBooks. Administrators can analyze compensation trends and resolve discrepancies, ensuring that employees are paid fairly while maintaining cost-effective operations.

The workforce management and optimization module streamlines care team operations by integrating performance tracking, automated task management, time tracking, training monitoring, and compensation alignment. By leveraging data-driven insights and automation, this system helps care teams work more efficiently, improve patient outcomes, and maintain high-quality care standards while supporting their professional growth.

In some embodiments, the system comprises a Population Health Management (PHM) module designed to help care teams monitor and improve the health of entire patient populations by tracking trends, identifying high-risk individuals, and optimizing preventive care strategies. This system enables proactive healthcare management, ensuring that interventions are tailored to the needs of different patient groups to improve long-term health outcomes.

One example function of the PHM module is risk stratification, which classifies patients into different risk levels based on their assessment scores, care plans, and health outcomes. The system automatically identifies high-risk patients-such as those with worsening depression or anxiety scores, or those missing critical interventions- and alerts care teams to intervene. It uses risk-scoring algorithms, patient history analytics, and customizable parameters to refine risk categorization, ensuring that care is personalized and targeted.

Another example feature is preventive care analytics, which leverages data insights to design early intervention programs that reduce the likelihood of chronic mental and/or physical health conditions. The system, through dashboards, helps care teams analyze population-level trends, such as the success rates of lifestyle interventions and health patterns across demographic groups. This allows care teams to implement preventive strategies—like lifestyle coaching or routine mental health check-ins—to support patients before their conditions worsen. System, 100 also facilitates automated reminders for preventive care activities, such as scheduling wellness sessions or lifestyle interventions.

For patients with chronic mental and/or physical health conditions, the chronic disease management component ensures continuous monitoring of adherence to treatment plans and/or therapy sessions. For example, the system 100 flags signs of non-compliance, such as missed appointments or deteriorating mental or physical health scores, so care teams can adjust interventions proactively. System 100 triggers alerts when adherence issues arise, and dashboards visually represent patient progress over time, allowing care managers to make data-driven adjustments to care plans.

The PHM module also provides real-time reporting and analytics to help care teams track population health and allocate resources effectively. Dashboards display risk distributions, intervention success rates, and adherence trends, allowing administrators to optimize staffing and funding decisions. These insights enable healthcare providers to prioritize high-risk patients, improve service delivery, and ensure that healthcare resources are used efficiently.

The PHM module in the system 100 enhances proactive care by integrating risk assessment, preventive care planning, and chronic disease management. By using data-driven insights and automated workflows, the system supports care teams in improving both individual patient outcomes and overall population health, leading to more effective and efficient healthcare management.

### Methods

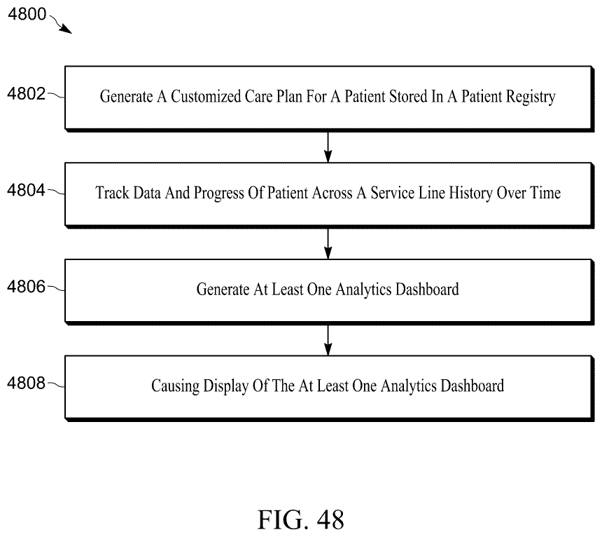

Referring to FIG. 48, a flowchart of a method 4800 for managing patient care workflows is provided. The method 4800 may be a computer-implemented process for managing patient care workflows operating on system 100. For example, the method 4800 may be carried out on system 100 using one or more processors 132 and memory 130.

At block 4802, the method 4800 may include generating a customized care plan for a patient stored in a patient registry. For example, the patient registry may include patient information such as demographic details, payer details, assigned care teams, and service line history, as described elsewhere herein.

In general, a customized care plan may tailor healthcare services based on the patient's specific medical history, diagnosis, treatment needs, and preferences. The patient registry (e.g., patient data repository 106) may act as a central repository where healthcare providers can access and update treatment plans, ensuring coordinated and consistent care. By structuring care plans in a digital format, the system 100 enhances efficiency and reduces the chances of miscommunication between healthcare teams.

In some embodiments, generating a customized care plan can include first performing an assessment/testing process for a service line for the patient. For example, the method 4800 may further include obtaining results of an administered psychological test and/or clinical data assessment and/or other patient assessments (e.g., PHQ-9, GAD-7, cognitive assessments, and/or lifestyle evaluations). The obtained results may be used to guide the care plan generation process.

At block 4804, following the creation of the care plan, the system 100 includes tracking data and progress of the patient across a service line history over time. The tracked data may include at least one service duration, session notes, and billing of a practitioner according to care provided to the patient. In some examples, the tracked data may further include various service-related data, including, but not limited to service duration, session notes, and billing information. Service duration helps in monitoring resource utilization, while session notes provide a record of diagnoses, treatment updates, and observations made by practitioners. Additionally, tracking billing information ensures transparency in financial transactions, allowing for proper reimbursement and compliance with healthcare regulations.

The tracking of the progress of the patient across a service line history over time may include tracking the journey of the patient. The patient journey may include phases such as an onboarding phase, an assessment phase, and a collaborative phase where patients engage with the personalized care plan while receiving treatments and lifestyle support, and an ongoing support and monitoring phase.

Example service lines may include, but are not limited to psychiatry service line, psychotherapy/care management service line, lifestyle psychiatry service line, assessment and testing service line, chronic care management (CCM) service line, and a referral care service line. The psychiatry service line may provide psychiatric treatment and medication management, overseen by the psychiatrist. This service line serves patients that have medication adjustments and psychiatric evaluations. The psychotherapy/care management (licensed psychotherapist) service line may focus on providing psychotherapy and managing care plans for patients, ensuring that both mental health and lifestyle interventions are integrated. The lifestyle psychiatry service line may be overseen by the health coach in collaboration with the psychotherapist. This service line focuses on integrating lifestyle interventions such as diet, exercise, and stress management into mental health care. The assessment and testing service line may be administered by the psychological testing technician and the clinical data specialist. This service line provides comprehensive assessments, including psychiatric evaluations (e.g., PHQ-9, GAD-7), cognitive assessments, and lifestyle evaluations. The results may guide the care planning process. The CCM service line may be responsible for coordinating long-term care and monitoring across other service lines. The CCM service line ensures that all care plans, assessments, and interventions are continuously managed and adjusted according to rules and clinician input. The referral care service line may be managed by a care coordinator. This service line handles patient referrals to external specialists, ensuring continuity of care across different providers (e.g., sleep specialists, nutritionists, etc.). The holistic patient care service line ensures that each patient is assigned a multidisciplinary care team that includes a psychiatrist, licensed psychotherapist (care manager), health coach, care coordinator, and clinical data specialist. these roles collaborate to create personalized care plans that integrate psychiatric treatment, psychotherapy, and lifestyle modifications. The testing and monitoring service line ensures regular assessments are performed through the testing and monitoring service line, overseen by the psychological testing technician and supported by the clinical data specialist. The assessments may include mental health tools like PHQ-9 and GAD-7, as well as lifestyle evaluations (e.g., sleep, stress, diet). The results may be shared with the care team for real-time adjustments to the care plan.

A CPT code (or other standardized procedure code or code representative of a service event or a line item for a healthcare procedure) integration service line or system may support claims management through the integration of CPT codes/codes for psychiatric evaluations, psychotherapy, patient self-education, and team-based care conferences. This may ensure that interventions are accurately tracked and billed.

At block 4806, once this data is collected, at least one analytics dashboard is generated. The at least one analytics dashboard may provide healthcare providers a centralized platform to visualize patient progress, service utilization trends, and financial metrics. This dashboard aids in informed decision-making by providing real-time insights into patient care effectiveness and operational efficiency. The dashboard may include one or more of: patient outcomes, referral effectiveness, and team performance metrics. Such content may be visualized in near real time based at least in part on the customized care plan, the tracked data, and the tracked progress of the patient.

The at least one analytics dashboard may include any combination of the dashboards described herein. For example, the system 100 may generate adherence dashboards that provide care teams with a visual representation of patient adherence over time, highlighting trends that may indicate worsening conditions or lapses in care. This allows the team to adjust treatment plans proactively.

The system 100 may further generate patient history dashboards for long-term monitoring of chronic conditions, ensuring that care teams can track patients' progress over months or years. These patient history dashboards provide a comprehensive view of a patient's mental health journey, physical health journey, or the like highlighting milestones, relapses, and recovery periods.

The system 100 may generate health metric dashboards that may obtain data from wearables, which may allow care managers to monitor patient health trends over time. Alerts can be triggered if a patient's health metrics fall outside of the defined thresholds, enabling early intervention.

The system 100 may generate patient engagement dashboards to provide a comprehensive view of the patient including, but not limited to adherence to lifestyle goals (e.g., track how consistently patients are meeting their lifestyle intervention goals, completing exercise sessions, logging food intake, etc.); completion of educational modules (e.g., assess how actively patients are engaging with educational resources and learning modules); and patient check-in rates (e.g., track how often patients engage with care teams or check-in through the patient portal.

The system 100 may generate patient trends dashboards to provide insights on one or more of patient improvement trends based on successive assessments, including mental health metrics, physical health metrics, and wellness goals; distribution of patients within specific score ranges for psychiatric conditions and wellness markers like stress reduction and mindfulness adherence; and outcomes linked to specific care plans, showing how medical interventions (e.g., psychiatric and physiological) and lifestyle interventions impact overall health.

The system 100 may generate dashboards that track wellness progress for patients, such as mindfulness course completion rates and engagement in lifestyle interventions, alongside psychiatric task completion. The system 100 may generate dashboards for providing insight into claims and/or billing. For example, a dashboard may be generated to offer insights into total claims submitted, percentage of claims paid, total revenue generated, and outstanding claims. This allows for data-driven decision-making around service lines and resource allocation.

The system 100 may generate role-specific dashboards that provide insights relevant to each user's role. For instance. For example, care managers will see task completion rates and patient progress, while health coaches will have dashboards focused on wellness goals and patient adherence to lifestyle changes. This ensures that team members are exposed to data that is directly relevant to their responsibilities, improving efficiency and reducing data overload, while blocking other extraneous data. The system 10 may generate dashboards to track system health and care team performance in real time. Such dashboards may track system health (e.g., workflow efficiency, data synchronization, task delays). This helps detect and resolve performance issues before they impact patient care. Tracking system health and care team performance can provide a proactive monitoring system to flag potential issues, such as delayed task escalations or data synchronization errors, before they impact patient care.

At block 4808, the method 4800 further includes causing display of the at least one analytics dashboard. The at least one analytics dashboard may be encrypted and accessible for view according to predefined patient permissions. For example, the at least one dashboard may be triggered (with permissions) to display key performance indicators/metrics, including patient outcomes, referral effectiveness, and team performance evaluations. These metrics help assess the success of care plans, determine the impact of referrals on treatment, and evaluate healthcare staff productivity. By presenting these insights, the system supports continuous improvement in patient care, provider efficiency, and overall healthcare service quality.

The encryption of such dashboards/information in the dashboards may safeguard sensitive patient information by implementing end-to-end encryption. Encryption ensures that patient records remain confidential and secure, protecting them from unauthorized access and tampering. This security measure enhances compliance with healthcare regulations such as HIPAA and fosters trust between patients and healthcare providers.

In some embodiments, the method 4800 further includes matching the at least one service duration and the care provided to the patient with one or more Current Procedural Terminology (CPT) codes. For example, the system 100 may track/monitor data over a patient journey or service duration and once care is provided, the system 100 tracks service duration, session notes, and billing information. The system, 100 then matches the provided services with the appropriate Current Procedural Terminology (CPT) codes to ensure accurate billing and compliance.

The system 100 may further provide access to a centralized repository to manage patient assessments, care plans, claims, and billing records. The method 4800 may further include receiving from a clinician interface, billable service entries representing the care provided to the patient and generating, based on the billable service entries, a corresponding Fast Healthcare Interoperability Resources (FHIR)-compliant claim containing patient details, provider information, and the CPT codes (or other standardized procedure code or code representative of a service event or a line item for a healthcare procedure) matched to the care provided to the patient. The claim may then be validated for completeness and compliance and submitted to a payer or clearinghouse. In response to the submission, the system 100 may receive and process any claim responses and update the claim registry.

By way of example, as care progresses, the system 100 continuously monitors the patient's progress across various service lines, and real-time analytics dashboards are generated based on the customized care plan, tracked data, and overall patient progress. These dashboards visualize metrics such as patient outcomes, referral effectiveness, and team performance, ensuring that care teams and administrators have a comprehensive view of operations. The system 100 also facilitates claims processing by receiving billable service entries from clinicians and generating corresponding FHIR-compliant claims. Each claim undergoes validation to ensure completeness and compliance before being submitted to payers or clearinghouses. To minimize rejections, the system cross-references CPT codes (or other standardized procedure code or code representative of a service event or a line item for a healthcare procedure) with payer policies. In cases where claims are rejected, the system identifies missing fields or incorrect codes, classifies errors based on severity, and either auto-corrects minor issues or flags manual corrections before resubmitting the claim for approval.

In addition to claims management, the system efficiently handles patient referrals by assigning them based on provider availability, specialization, and patient needs. It also ensures that pending referrals are followed up on and flags unresolved cases for manual intervention. Real-time notifications are sent to specialists, referring clinicians, and patients, keeping all stakeholders informed about updates. Once claims are approved, the system generates invoices, matches received payments with the corresponding invoices, and identifies adjustments or discrepancies. Any outstanding balances are flagged for escalation and further action.

The system 100 may further assess and classify rejected claims based on error severity, distinguishing between minor auto-correctable errors and manually correctable errors. The system 100 may then correct rejected claims based on the classifying. The correction process may include automatically generating missing data for the missing fields or performing one or more suggested manual modifications classified as a manually correctable error. Upon completion of the correction, the system 100 may resubmit corrected claims to payers or clearing houses after validation.

In some embodiments, the method 4800 may further include receiving patient referrals and storing, updating and managing the patient referrals within the centralized repository. The method 4800 may further include assigning one or more of the received patient referrals based on provider availability, specialization, and patient needs, triggering follow-ups for pending referrals and flagging unresolved cases for manual intervention associated with one or more of the received patient referrals. The method 4800 may further include providing a real-time notification that alerts at least one of an assigned specialist, referring clinician, and the patient of the triggered follow-ups.

Task management is another component of the process flow of method 4800. The system 100 assigns tasks to clinicians based on workload balancing and availability, ensuring equitable distribution. If tasks become overdue, they are flagged and reassigned as needed. Real-time updates on pending patient referrals, claims, and follow-ups are continuously provided, with alerts notifying users of pending tasks, due dates, and urgent follow-ups. Furthermore, before sharing any patient data, the system verifies patient consent preferences using the patient registry. Unauthorized access attempts are blocked, and audit logs are generated to ensure regulatory compliance.

Throughout this workflow, compliance auditing tools track user activities, maintain adherence to regulatory standards such as HIPAA and HITECH, and generate necessary compliance reports. As illustrated in FIG. 48, the system integrates automation, analytics, and security measures to optimize patient care, streamline administrative workflows, and ensure operational transparency. This comprehensive process ensures efficiency, regulatory adherence, and improved patient outcomes.

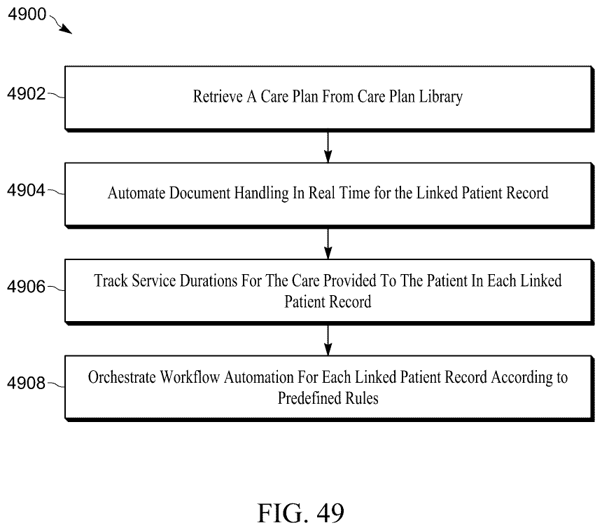

Referring to FIG. 49, a method 4900 for managing patient care plans, automated document handling, tracking service duration, and orchestrating workflow automation based on predefined rules is provided.

At block 4902, the method 4900 begins with retrieving a care plan from a care plan library 154. The care plan library 154 serves as a repository containing standardized or customized care plans based on various medical conditions, treatment protocols, and patient needs. By selecting an appropriate care plan from this library, healthcare providers ensure that each patient receives a structured and evidence-based treatment approach. This step reduces the time utilized for care planning and promotes consistency in treatment across multiple cases.

At block 4904, the system 100 automates document handling in real-time for the linked patient record. This automation ensures that medical documents, including prescriptions, lab reports, consultation notes, and treatment histories, are automatically updated, categorized, and linked to the corresponding patient record. Real-time document handling minimizes manual data entry errors, streamlines administrative tasks, and ensures that all relevant information is readily accessible to healthcare professionals for informed decision-making.

At block 4906, the system 100 tracks service durations for the care provided to the patient in each linked patient record. Tracking service durations allows healthcare providers and administrators to monitor the time spent on various medical procedures, consultations, and therapies. This information is valuable for evaluating treatment efficiency, optimizing resource allocation, and ensuring compliance with healthcare billing regulations. Service duration tracking also supports performance analysis by providing insights into how different treatments impact patient outcomes over time.

At block 4908, the method 4900 involves orchestrating workflow automation for each linked patient record according to predefined rules. Workflow automation ensures that care processes are executed in a structured manner, reducing delays and inconsistencies in treatment delivery. Predefined rules can include guidelines for treatment escalation, medication reminders, follow-up appointment scheduling, and compliance checks. By automating workflows, healthcare providers can enhance coordination, improve patient engagement, and achieve better healthcare outcomes while minimizing administrative overhead.

The care plan library 154 contains pre-defined templates that are dynamically customized based on patient-specific data stored in the patient registry. Once the care plan is retrieved, the system automates document handling in real-time, ensuring that all relevant records—including care plans, assessment data, progress reports, and claims records—are generated or updated as necessary.

For both method 4800 and method 4900, as the patient receives care, the system 100 tracks service durations, logging the time spent by healthcare providers and associating it with standardized billing codes. The time tracking and billing module records these billable minutes to ensure accurate reimbursement and compliance with payer requirements. The clinical workflow engine then orchestrates workflow automation, which involves automatically generating claims based on the tracked service durations and matching them with the appropriate billing codes.

Once claims are generated, the clinical workflow engine processes them according to predefined rules. This includes validating claims for accuracy, tracking associated care referrals, and escalating tasks if issues arise during processing. Additionally, the system generates real-time reports containing referral tracking metrics and team performance analytics to enhance operational oversight.

To maintain compliance, the clinical workflow engine establishes an audit trail, recording all workflow automation activities. This audit trail is stored within the patient registry and practitioner registry, ensuring regulatory adherence and providing administrators with traceable logs for compliance tracking.

The system also submits claims in Fast Healthcare Interoperability Resources (FHIR)-compliant formats to external payer systems. Before submission, claims are verified against payer-specific rules, such as Current Procedural Terminology (CPT) code restrictions and Medically Unlikely Edits (MUE) limits, to minimize errors. Payer responses are then received, categorized, and processed for approval tracking, resubmissions, and financial reconciliation.

If claims are rejected, the clinical workflow engine categorizes them based on error types and triggers automated resubmission workflows after generating corrected claims. The error resolution process involves identifying missing fields, incorrect CPT codes (or other codes), or payer-specific compliance issues. The system classifies rejected claims by error severity, distinguishing between minor auto-correctable issues and major errors requiring manual review. Auto-filling missing data or suggesting manual modifications ensures efficiency in claim corrections before resubmission.

In addition to claims processing, the system handles patient referrals, which are stored, updated, and managed within a centralized database. Referrals are dynamically assigned based on provider availability, specialization, and patient needs. To prevent delays, the system triggers follow-ups for pending referrals and flags unresolved cases for manual intervention. Real-time alerts notify assigned specialists, referring clinicians, and patients about referral status updates. If referrals exceed a predefined wait time, the system generates clinician notifications for expedited handling.

The method 4800 may further include financial reconciliation. Once a claim is approved, the system creates invoices, reconciles payments using Explanation of Benefits (EOB) data, and processes denials or underpayments. If unpaid invoices remain, the system flags them and triggers automated follow-ups for overdue payments, ensuring streamlined revenue cycle management.

As used herein, the term “CPT code” may refer to a United States insurance and or healthcare system of codes or other standardized procedure code or code representative of a service event or a line item for a healthcare procedure. Other codes are of course possible and may directly replace the term “CPT code” with a different healthcare system of codes or other standardized procedure code or code representative of a service event or a line item for a healthcare procedure represented in another country. While the United States healthcare and insurance systems utilize particular codes/CPT codes, other jurisdictions use different procedures and/or billing codes, which are also contemplated to work with the systems and methods described herein.

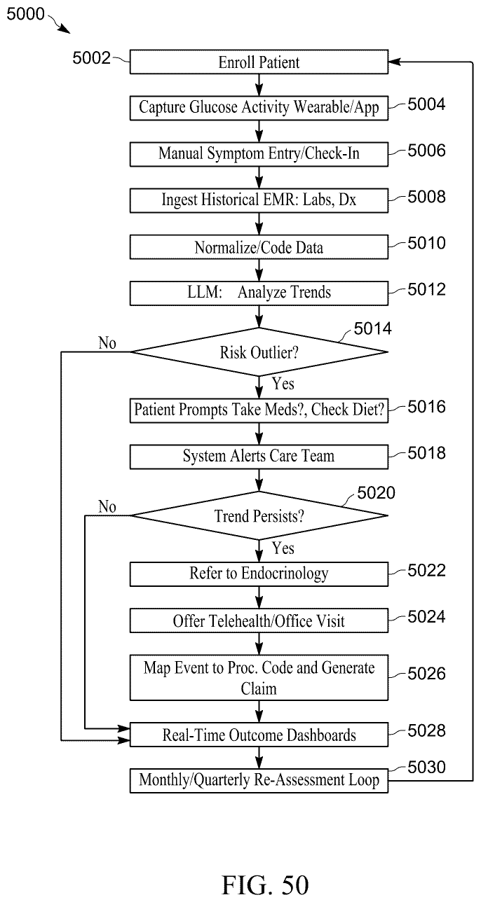

FIG. 50 illustrates an example flow chart of a method for utilizing the clinical assessment system 100 for a patient managing diabetes. The method 5000 describes an intelligent, closed-loop system for patient monitoring and intervention, particularly suitable for managing chronic conditions such as diabetes. The method 5000 begins with patient enrollment (step S002), wherein the patient is registered into the platform and linked with relevant health data sources as described elsewhere herein. Following enrollment, the system 100 initiates data capture through a wearable device or mobile application to continuously monitor glucose activity (step S004). Patients can optionally manually input symptoms or conduct self-check-ins using an interface designed for real-time data supplementation (step S006).

To provide a comprehensive clinical view, the system 100 also ingests historical electronic medical records (EMRs), including laboratory results and diagnostic codes (step S008). The collected data—both real-time and historical—are normalized and coded into a standardized format for processing (step S010). A large language model (LLM) or similar AI-based engine then analyzes this unified dataset to detect trends, anomalies, or clinical red flags (step S012).

Upon completing the trend analysis, the system 100 determines whether the patient qualifies as a risk outlier (step S014). If no risk is detected, the system 100 continues its monitoring cycle. If a risk is identified, the system 100 initiates a patient-directed intervention by prompting the user to confirm medication adherence, check dietary compliance, or review lifestyle factors (step S016). If further action is warranted, the system 100 may optionally escalate the issue by alerting the care team (step S018). If a risk is not identified, the system 100 may jump to step S028 to generate an update, data, or message about the lack of risk.

A subsequent check evaluates whether the clinical trend persists over time (step S020). If the trend stabilizes, the patient returns to the general monitoring flow. However, if the trend continues, the patient is referred to a specialist, such as an endocrinologist (step S022), and offered either a telehealth session or an in-office consultation (step S024).

Once the event is clinically processed, the system 100 maps the event to the appropriate procedural code for billing purposes and generates a claim associated with the event (step S026). Outcomes and system actions are visualized through real-time dashboards for clinicians and administrators (step S028). Finally, the system includes a structured re-assessment loop (step S030), scheduled on a monthly or quarterly basis, to evaluate ongoing trends and update patient care plans accordingly.

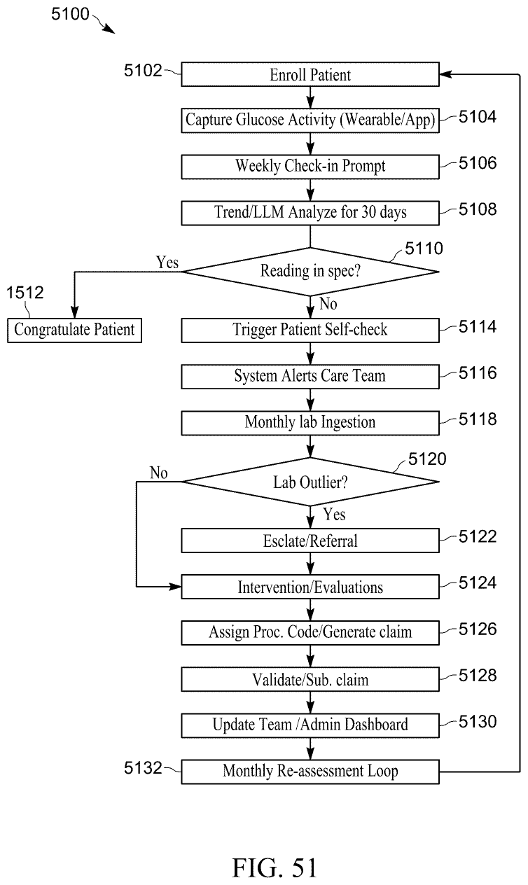

FIG. 51 illustrates an example flow chart of a method 5100 for utilizing the clinical assessment system 100 for a patient managing a chronic disease. The method 5100 illustrates a patient monitoring and intervention system designed to manage glucose levels (or other measurable health metric) and related health outcomes through a hybrid of wearable technology, AI-based analysis, and clinical workflows. The process 5100 begins at step S102 with the enrollment of a patient into the program. Once enrolled, the system 100 captures glucose activity data (or other measurable/detectable health metric) using a wearable device or mobile application (step S104). To ensure patient engagement and consistent data entry, the system 100 may issue an optional weekly check-in prompt (step S106). In some embodiments, the check-in prompt is hourly, daily, bi-weekly, monthly, or yearly instead of weekly.

The data gathered from the wearable and patient check-ins are then analyzed over a 30-day period using a trend analysis module powered by a large language model (LLM) or similar AI tool (step S108). After this analysis, the system 100 determines whether the patient's glucose readings are within the expected clinical range (step S110). If the readings are within specification, the system provides positive feedback to the patient (step 1512), reinforcing adherence and successful management. In this example, the positive feedback is a congratulatory message that may be provided in a dashboard and/or via messaging to a mobile device or the like.

However, if the readings fall outside the target range, the system 100 initiates a self-check prompt to the patient (step S114), encouraging the patient to assess medication adherence, diet, and/or lifestyle factors. The system 100 may also alert the care team (step S116) to facilitate early intervention. Monthly laboratory data are also ingested into the system 100 (step S118), allowing for comprehensive, real-time assessment of the patient's condition.

The system 100 then evaluates the lab data to detect any clinical outliers (step S120). If no anomalies are detected, the system 100 re-enters the monitoring cycle to continue monitoring the patient. If an outlier is detected, the system 100 triggers an escalation protocol (step S122), leading to clinical intervention and patient evaluations (step S124). Relevant procedural codes are assigned and used to generate billing claims (step S126), which are then validated and submitted (step S128). Once the administrative steps are completed, the care team and administrative dashboards are updated (step S130) to reflect the patient's status and intervention history.

Finally, the system 100 enters a monthly re-assessment loop (step S132), enabling continuous tracking, adjustment, and optimization of patient care. This cyclical and intelligent workflow supports proactive disease management and ensures that both patients and healthcare providers are engaged through automated alerts, data insights, and seamless coordination.

The systems and methods described herein can be embodied and/or implemented at least in part as a machine configured to receive a computer-readable medium storing computer-readable instructions. The instructions are preferably executed by computer-executable components preferably integrated with the system and one or more portions of the processor 132 on the clinical assessment system 100 and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

For instance, the processor 132, as described in FIG. 1B, may include specialized accelerators for machine learning tasks, such as GPUs or TPUs, to execute computationally intensive operations like training and inference for the trained LLM 102. These processors may retrieve the instructions from the memory 130, where the instructions are stored in non-transitory storage media, and execute tasks such as normalizing patient data, performing contextual analysis, and generating clinical insights.

Additionally, the instructions may define the workflows of various modules such as the clinical workflow engine 108, care management module 124, and security and compliance module 1922, ensuring that each component performs its designated functions in an integrated manner. The computer-readable medium may also include software libraries and frameworks enabling the system to interface with external devices, such as wearable devices 138, patient interface 110, or clinician interface 112.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used in the description and claims, the singular form “a”, “an” and “the” include both singular and plural references unless the context clearly dictates otherwise. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-9. (canceled)

10. A computer-implemented system for managing patient care workflows, the system comprising:
a patient registry comprising patient data and configured to maintain patient demographic details, insurance information, and assigned care teams;
a practitioner registry comprising a plurality of practitioners adapted to provide care to patients in the patient registry;
a care plan library storing a plurality of care plan templates configured for dynamic customization for patients in the patient registry;
a time tracking and billing module configured to receive practitioner time and generate bills for the care provided to the patients in the patient registry;
a clinical workflow engine configured to generate and process claims;
a processor; and
memory communicably coupled to the processor, wherein the memory stores processor-executable instructions, which when executed by the processor, cause the processor to trigger the clinical workflow engine to link patient records with provider data stored in the practitioner registry to define care team assignments;
for each linked patient record:
retrieve a care plan from the care plan library;
automate document handling in real time for the linked patient record to generate or update the care plan, assessment data, progress reports, and claims records associated with the linked patient record;
track service durations for the care provided to the patient in each linked patient record, wherein billable minutes are associated with standardized billing codes; and
orchestrate workflow automation for each linked patient record according to predefined rules, the orchestration comprising generating claims according to the standardized billing codes based on the tracked service durations.

11. The system of claim 10, wherein the processor is further configured to trigger the clinical workflow engine to perform, according to the predefined rules:
processing of the generated claims,
escalating tasks associated with processing the generated claims,
tracking care referrals associated with the generated claims, and
generating real-time reports comprising referral tracking metrics and performance analytics.

12. The system of claim 10, wherein the processor is further configured to trigger the clinical workflow engine to maintain an audit trail for each orchestrated workflow automation, the audit trail being configured for use in compliance tracking of records in the patient registry and the practitioner registry.

13. The system of claim 10, wherein the processor is further configured to trigger the clinical workflow engine to:
submit the generated claims in Fast Healthcare Interoperability Resources (FHIR)-compliant formats to external payer systems;
verify the generated claims against payer-specific rules, including Current Procedural Terminology (CPT) code restrictions and Medically Unlikely Edits (MUE) limits; and
receive, categorize, and process payer responses, facilitating approval tracking, resubmissions for rejected claims, and financial reconciliation.

14. The system of claim 13, wherein the processor is further configured to trigger the clinical workflow engine to:
identify rejected claims,
categorize the rejected claims based on error type, and
trigger automated resubmission workflows after generating corrected claims.

15. The system of claim 14, wherein generating the corrected claims comprises:
identifying in the rejected claims, missing fields, incorrect CPT codes, or payer-specific compliance issues;
classifying rejected claims based on error severity, distinguishing between minor auto-correctable errors and major issues requiring manual review;
auto-filling missing data or suggesting manual modifications; and
resubmitting the corrected claims to payers or clearinghouses after validation.

16. The system of claim 10, wherein the processor is further configured to trigger the clinical workflow engine to:
store, update, and manage patient referrals within a centralized database;
dynamically assign referrals based on provider availability, specialization, and patient needs;
trigger follow-ups for pending referrals and flag unresolved cases for manual intervention; and
alert assigned specialists, referring clinicians, and patients of referral status updates.

17. The system of claim 16, wherein the processor is further configured to trigger the clinical workflow engine to notify clinicians when pending referrals exceed a predefined wait time.

18. The system of claim 10, wherein the processor is further configured to trigger the clinical workflow engine to:
create invoices based on approved claims, including billed amounts, due dates, and payer details;
reconcile received payments with corresponding invoices using Explanation of Benefits (EOB) data;
process partial payments, denials, and underpayments, updating financial records accordingly; and
flag unpaid invoices and trigger automated follow-ups for overdue payments.

19. (canceled)

20. (canceled)

21. (canceled)

22. The computer-implemented system of claim 10, wherein the processor is further configured to:
submit a generated claim using a clearinghouse interface,
receive an acknowledgment prior to adjudication, and
parse the acknowledgment to identify submission errors and trigger correction workflows.

23. The computer-implemented system of claim 10, wherein operations associated with the claims processing are gated by role-based access controls and protected by encryption in transit and at rest.

24. The computer-implemented system of claim 10, wherein generating the claims according to the standardized billing codes further comprises mapping the claims to the standardized billing codes using event-based triggers.

25. A non-transitory computer-readable storage medium storing executable program instructions that, when executed by a processor, cause the processor to perform operations comprising:
accessing a patient registry comprising patient data and configured to maintain patient demographic details, insurance information, and assigned care teams;
accessing a practitioner registry comprising a plurality of practitioners adapted to provide care to patients in the patient registry;
accessing a care plan library storing a plurality of care plan templates configured for dynamic customization for patients in the patient registry;
accessing a time tracking and billing module configured to receive practitioner time and generate bills for the care provided to the patients in the patient registry;
accessing a clinical workflow engine configured to generate and process claims;
accessing memory communicably coupled to the processor, wherein the memory stores processor-executable instructions, which when executed by the processor, cause the processor to trigger the clinical workflow engine to link patient records with provider data stored in the practitioner registry to define care team assignments;
for each linked patient record:
retrieve a care plan from the care plan library;
automate document handling in real time for the linked patient record to generate or update the care plan, assessment data, progress reports, and claims records associated with the linked patient record;
track service durations for the care provided to the patient in each linked patient record, wherein billable minutes are associated with standardized billing codes; and
orchestrate workflow automation for each linked patient record according to predefined rules, the orchestration comprising generating claims according to the standardized billing codes based on the tracked service durations.

26. The non-transitory computer-readable storage medium of claim 25, wherein the processor is further configured to trigger the clinical workflow engine to perform, according to the predefined rules:
processing of the generated claims,
escalating tasks associated with processing the generated claims,
tracking care referrals associated with the generated claims, and
generating real-time reports comprising referral tracking metrics and performance analytics.

27. The non-transitory computer-readable storage medium of claim 25, wherein the processor is further configured to trigger the clinical workflow engine to maintain an audit trail for each orchestrated workflow automation, the audit trail being configured for use in compliance tracking of records in the patient registry and the practitioner registry.

28. The non-transitory computer-readable storage medium of claim 25, wherein the processor is further configured to trigger the clinical workflow engine to:
identify rejected claims,
categorize the rejected claims based on error type, and
trigger automated resubmission workflows after generating corrected claims.

29. The non-transitory computer-readable storage medium of claim 28, wherein generating the corrected claims comprises:
identifying in the rejected claims, missing fields, incorrect CPT codes, or payer-specific compliance issues;
classifying rejected claims based on error severity, distinguishing between minor auto-correctable errors and major issues requiring manual review;
auto-filling missing data or suggesting manual modifications; and
resubmitting the corrected claims to payers or clearinghouses after validation.

30. The non-transitory computer-readable storage medium of claim 25, wherein the processor is further configured to trigger the clinical workflow engine to:
store, update, and manage patient referrals within a centralized database;
dynamically assign referrals based on provider availability, specialization, and patient needs;
trigger follow-ups for pending referrals and flag unresolved cases for manual intervention; and
alert assigned specialists, referring clinicians, and patients of referral status updates.

31. The non-transitory computer-readable storage medium of claim 25, wherein the processor is further configured to trigger the clinical workflow engine to:
create invoices based on approved claims, including billed amounts, due dates, and payer details;
reconcile received payments with corresponding invoices using Explanation of Benefits (EOB) data;
process partial payments, denials, and underpayments, updating financial records accordingly; and
flag unpaid invoices and trigger automated follow-ups for overdue payments.

32. The non-transitory computer-readable storage medium of claim 25, wherein the processor is further configured to:
submit a generated claim using a clearinghouse interface,
receive an acknowledgment prior to adjudication, and
parse the acknowledgment to identify submission errors and trigger correction workflows.

33. The non-transitory computer-readable storage medium of claim 25, wherein operations associated with the claims processing are gated by role-based access controls and protected by encryption in transit and at rest.

34. The non-transitory computer-readable storage medium of claim 25, wherein generating the claims according to the standardized billing codes further comprises mapping the claims to the standardized billing codes using event-based triggers.

35. A computer-implemented method for managing patient care workflows, the method having access to a processor and memory storing executable program instructions that, when executed by the processor, cause the processor to perform operations comprising:
accessing a patient registry comprising patient data and configured to maintain patient demographic details, insurance information, and assigned care teams;
accessing a practitioner registry comprising a plurality of practitioners adapted to provide care to patients in the patient registry;
accessing a care plan library storing a plurality of care plan templates configured for dynamic customization for patients in the patient registry;
accessing a time tracking and billing module configured to receive practitioner time and generate bills for the care provided to the patients in the patient registry;
accessing a clinical workflow engine configured to generate and process claims;
accessing memory communicably coupled to the processor, wherein the memory stores processor-executable instructions, which when executed by the processor, cause the processor to trigger the clinical workflow engine to link patient records with provider data stored in the practitioner registry to define care team assignments;
for each linked patient record:
retrieve a care plan from the care plan library;
automate document handling in real time for the linked patient record to generate or update the care plan, assessment data, progress reports, and claims records associated with the linked patient record;
track service durations for the care provided to the patient in each linked patient record, wherein billable minutes are associated with standardized billing codes; and
orchestrate workflow automation for each linked patient record according to predefined rules, the orchestration comprising generating claims according to the standardized billing codes based on the tracked service durations.

36. The computer-implemented method of claim 35, wherein the processor is further configured to trigger the clinical workflow engine to perform, according to the predefined rules:
processing of the generated claims,
escalating tasks associated with processing the generated claims,
tracking care referrals associated with the generated claims, and
generating real-time reports comprising referral tracking metrics and performance analytics.

37. The computer-implemented method of claim 35, wherein the processor is further configured to trigger the clinical workflow engine to maintain an audit trail for each orchestrated workflow automation, the audit trail being configured for use in compliance tracking of records in the patient registry and the practitioner registry.

38. The computer-implemented method of claim 35, wherein the processor is further configured to trigger the clinical workflow engine to:
identify rejected claims,
categorize the rejected claims based on error type, and
trigger automated resubmission workflows after generating corrected claims.

39. The computer-implemented method of claim 35, wherein the processor is further configured to trigger the clinical workflow engine to:
store, update, and manage patient referrals within a centralized database;
dynamically assign referrals based on provider availability, specialization, and patient needs;
trigger follow-ups for pending referrals and flag unresolved cases for manual intervention; and
alert assigned specialists, referring clinicians, and patients of referral status updates.

40. The computer-implemented method of claim 35, wherein the processor is further configured to:
submit a generated claim using a clearinghouse interface,
receive an acknowledgment prior to adjudication, and
parse the acknowledgment to identify submission errors and trigger correction workflows.

41. The computer-implemented method of claim 35, wherein operations associated with the claims processing are gated by role-based access controls and protected by encryption in transit and at rest.

42. The computer-implemented method of claim 35, wherein generating the claims according to the standardized billing codes further comprises mapping the claims to the standardized billing codes using event-based triggers.
